(19) United States
(12) Patent Application Publication
(10) Pub. No.: US 2025/0212744 A1
(43) Pub. Date: Jul. 3, 2025

(54) COMPOSITIONS AND METHODS COMPRISING PLANTS WITH SELECT FATTY ACID PROFILE

(71) Applicant: Benson Hill, Inc., St. Louis, MO (US)

(72) Inventors: Hao Zhou, St. Louis, MO (US); Herbert Wolfgang Goettel, St. Louis, MO (US); Avjinder Singh Kaler, St. Louis, MO (US); Logan Duncan, St. Louis, MO (US)

(73) Assignee: Benson Hill, Inc., St. Louis, MO (US)

(21) Appl. No.: 18/853,521

(22) PCT Filed: Apr. 4, 2023
(86) PCT No.: PCT/IB2023/053406

§ 371 Date: Oct. 2, 2024

Related U.S. Application Data
(60) Provisional application No. 63/327,047, filed on Apr. 4, 2022.

(51) Int. Cl. A01H 1/00; A01H 5/10; A01H 6/54; C12Q 1/6895

(52) U.S. Cl. CPC A01H 1/104; A01H 5/10; A01H 6/542; C12Q 1/6895; C12Q 2600/13; C12Q 2600/156

(57) ABSTRACT

Provided herein are methods and compositions for producing soybean plants having a select fatty acid composition, e.g., high saturated fatty acid, high monounsaturated fatty acid, and/or low polyunsaturated fatty acid content, using marker-assisted selection. The disclosure further provides methods and compositions for introgressing one or more quantitative trait loci (QTLs) associated with such phenotype. Plants produced according to the methods provided herein, and soybean oil having high saturated fatty acid, high monounsaturated fatty acid, and/or low polyunsaturated fatty acid content are also provided.

### RELATED APPLICATIONS

This application claims priority to U.S. Provisional Application No. 63/327,047 filed on Apr. 4, 2022, the content of which is incorporated herein by reference in its entirety.

### FIELD OF THE INVENTION

This disclosure relates generally to the field of agricultural biotechnology. More specifically, this disclosure relates to soybean oil, methods and compositions for producing soybean plants or seeds with select fatty acid profile, and plants, oil, and compositions produced thereby.

### SEQUENCE LISTING

This application contains a Sequence Listing which is submitted herewith in electronically readable format. The Sequence Listing file was created on Apr. 3, 2023, is named “B88552_1380_SL.xml” and its size is 45.7 kb. The entire contents of the Sequence Listing file are incorporated by reference herein.

### BACKGROUND OF THE INVENTION

Many plants store and utilize fatty acids as an energy source. Plant fatty acids and lipids (i.e., fats and oil having fatty acids and glycerin as compositions) are widely utilized for food, cosmetic, or industrial purposes. The specific performance and health attributes of edible oils is determined largely by their fatty acid composition. Plant oils derived from commercial plant varieties are composed primarily of palmitic (16:0), stearic (18:0), oleic (18:1), linoleic (18:2) and linolenic (18:3) acids, wherein the first and second numbers represent the number of carbons and the number of double bonds, respectively, in the fatty acid chain. Palmitic and stearic acids are 16- and 18-carbon-long, saturated fatty acids, respectively. Oleic, linoleic and linolenic are 18-carbon-long, unsaturated fatty acids containing one, two, and three double bonds, respectively.

Oil with higher polyunsaturated fatty acid content commonly provides more health benefits. On the other hand, oil with higher saturated fatty acid content has longer shelf life and higher melting temperature, providing a wide applicability in food and industrial processing. For example, oil comprising high saturated/monounsaturated and low polyunsaturated fatty acid composition, e.g., palm oil [comprising approximately 50% saturated fatty acids (e.g., palmitic acid, stearic acid), approximately 40% monounsaturated fatty acids (e.g., oleic acid), and approximately 10% polyunsaturated fatty acids (e.g., linoleic acid and linolenic acid)] is stable at high temperature and is suited for cooking, frying, and a variety of industrial use. The high melting temperature makes such plant oil (e.g., palm oil) a cost-effective replacement for animal fats (e.g., margarine preparation). Polyunsaturated fatty acid content in plant oil can be reduced by fully or partially hydrogenating unsaturated fatty acid-containing plant oil. However, hydrogenation produces trans fatty acid (trans fat), which triggers adverse health consequences, e.g., increased risk of atherosclerosis, when consumed. On the other hand, non-hydrogenated plant oil comprising high saturated/monounsaturated and low polyunsaturated fatty acid content with low (e.g., less than 1%) trans-fat content (e.g., palm oil) can be a healthier alternative to hydrogenated or partially hydrogenated fat.

While palm oil or oil having similar composition offers useful characteristics and broad applicability with health benefits, production of palm oil has been causing destruction of the natural forests and loss of habitats and food for wild animals including endangered species. Accordingly, alternative oil and oil products comprising a high saturated fatty acid, high monounsaturated fatty acid, and/or low polyunsaturated fatty acid content, and methods of producing the same, for example from soybean, can offer significant commercial, environmental, and/or health advantages.

### SUMMARY OF THE INVENTION

The present disclosure identifies and validates novel quality trait loci (QTLs) associated with high saturated fatty acid (e.g., palmitic and/or stearic acid), high monounsaturated fatty acid (e.g., oleic acid), and/or low polyunsaturated fatty acid (e.g., linoleic and linolenic acid) phenotype (referred to as a “HPHOLL” phenotype herein) in soybean, and provides molecular markers, e.g., saturated molecular markers and unsaturated molecular markers, linked to these HPHOLL loci. The present disclosure provides compositions and methods for producing a population of soybean plants or seeds comprising HPHOLL characteristics, and compositions and methods for introgressing a quantitative trait locus (QTL) associated with HPHOLL characteristics. Further provided herein are HPHOLL soybean oil and HPHOLL soybean varieties.

In one aspect, the present disclosure provides a method of producing a population of soybean plants or seeds comprising high palmitic acid or high stearic acid content relative to a control plant or seed, said method comprising: (a) genotyping a first population of soybean plants or seeds for the presence of at least one saturated marker associated with high palmitic acid or high stearic acid content, wherein the at least one saturated marker is within 20 centimorgans of at least one saturated quantitative trait locus (QTL) associated with high palmitic acid and/or high stearic acid content located within a genomic region 3567986-9738629 of chromosome 8 of a soybean genome; (b) selecting from the first population one or more soybean plants or seeds comprising one or more alleles comprising said at least one saturated marker associated with high palmitic acid or high stearic acid content; (c) producing a second population of progeny soybean plants or seeds from the one or more soybean plants or soybean seeds selected from the first population, wherein the second population of progeny soybean plants or seeds comprises one or more alleles comprising said at least one saturated marker, and wherein the second population of progeny soybean plants or seeds comprises high palmitic acid or high stearic acid content relative to a control population. In some embodiments, the at least one saturated QTL associated with high palmitic acid or high stearic acid content is one or more of Gm08:063500, Gm08:045000, Gm08:057400, Gm08:072100, Gm08:083900, Gm08:084300, Gm08:092100, and Gm08:126400. In some embodiments, said at least one saturated QTL comprises at least one single nucleotide polymorphisms (SNP), and the at least one saturated marker comprises an allele of the at least one SNP. In some embodiments, the at least one SNP is a G or an A at position 4879302, a T or a C at position 3567986, a T or a C at position 4416970, an A or a T at position 5521970, an A or a T at position 6333332, a T or a C at position 6357981, a T or a C at position 6958927, and/or an A or a G at position 9738629 of chromosome 8 the soybean genome, wherein the G at position 4879302, the T at position 3567986, the T at position 4416970, the A at position 5521970, the A at position 6333332, the T at position 6357981, the T at position 6958927, or the A at position 9738629 of chromosome 8 of the soybean genome is associated with high palmitic acid content. In specific embodiments, the at least one SNP is a G or an A at position 4879302 of chromosome 8 and/or a T or a C at position 6357981 of chromosome 8 of the soybean genome, wherein the G at position 4879302 of chromosome 8 or the T at position 6357981 of chromosome 8 is associated with high palmitic acid content.

In some embodiments, the genotyping comprises analyzing the at least one SNP or a haplotype. In some embodiments, the genotyping comprises analyzing the at least one SNP or the haplotype using an oligonucleotide probe comprising at least 15 nucleotides, wherein the oligonucleotide probe has at least 90% sequence identity to a sequence of the same number of contiguous nucleotides of a sense or antisense DNA strand in a region comprising or adjacent to the at least one SNP in the soybean genome. In some embodiments, the oligonucleotide probe comprises a nucleic acid sequence of any one of SEQ ID NOs: 1, 2, 5, 6, 9, 10, 13, 14, 17, 18, 21, 22, 25, 26, 29, and 30; or a nucleic acid sequence complementary to a nucleic acid sequence of any one of SEQ ID NOs: 1, 2, 5, 6, 9, 10, 13, 14, 17, 18, 21, 22, 25, 26, 29, and 30.

In some embodiments, the genotyping comprises analyzing the at least one SNP or the haplotype using a first primer and a second primer each comprising at least 15 nucleotides, wherein the first primer has at least 90% sequence identity to a sequence of the same number of contiguous nucleotides of a sense DNA strand of a region comprising or adjacent to the at least one SNP, and the second primer has at least 90% sequence identity to a sequence of the same number of contiguous nucleotides of an antisense DNA strand of the region comprising or adjacent to the at least one SNP. In some embodiments, the first and second primers comprise any one pair of: (i) nucleic acid sequences of SEQ ID NOs: 3 and 4; (ii) nucleic acid sequences of SEQ ID NOs: 7 and 8; (iii) nucleic acid sequences of SEQ ID NOs: 11 and 12; (iv) nucleic acid sequences of SEQ ID NOs: 15 and 16; (v) nucleic acid sequences of SEQ ID NOs: 19 and 20; (vi) nucleic acid sequences of SEQ ID NOs: 23 and 24; (vii) nucleic acid sequences of SEQ ID NOs: 27 and 28; and (viii) nucleic acid sequences of SEQ ID NOs: 31 and 32.

In some embodiments, the second population of progeny soybean plants or seeds comprises at least about 4% increase in palmitic acid content or at least about 0.5% increase in stearic acid content compared to a control population of soybean plants or seeds. In some embodiments, the second population of progeny soybean plants or seeds comprises oil having a palmitic acid content of about 15% to about 30% or a stearic acid content of about 2.5% to about 3.5%.

In some embodiments, the method provided herein comprises: (a) genotyping the first population of soybean plants or seeds for the presence of (i) said at least one saturated marker associated with high palmitic acid or high stearic acid content and (ii) at least one unsaturated marker associated with high oleic acid, low linoleic acid, and/or low linolenic acid content, wherein the at least one unsaturated marker is within 20 centimorgans of at least one unsaturated QTL associated with high oleic acid, low linoleic acid, and/or low linolenic acid content; (b) selecting from the first population one or more soybean plants or seeds comprising one or more alleles comprising (i) the at least one saturated marker associated with high palmitic acid and/or high stearic acid content and (ii) the at least one unsaturated marker associated with high oleic acid, low linoleic acid, and/or low linolenic acid content; and (c) producing a second population of progeny soybean plants or seeds from the one or more soybean plants or soybean seeds selected from the first population, wherein the second population of progeny soybean plants or seeds comprises one or more alleles comprising (i) said at least one saturated marker associated with high palmitic acid and/or high stearic acid content and (ii) said at least one unsaturated marker associated with high oleic acid, low linoleic acid, and/or low linolenic acid content, wherein the second population of progeny soybean plants or seeds comprises high palmitic acid, high oleic acid, low linoleic acid, and/or low linolenic acid content relative to a control population.

In some embodiments, said at least one unsaturated QTL associated with high oleic acid, low linoleic acid, and/or low linolenic acid content is _{Gm10:50014440}, Gm20:35318088, Gm14:45937922, Gm14:45937935, and/or Gm02:41422213. In some embodiments, said at least one unsaturated marker associated with high oleic acid, low linoleic acid, and/or low linolenic acid content is located in Glyma.10G278000, Glyma.20G111000, Glyma.14G194300, and/or Glyma.02G227200 of the soybean genome. In some embodiments, said at least one unsaturated QTL comprises at least one SNP, and the at least one saturated marker comprises an allele of the at least one SNP. In some embodiments, the at least one SNP is an A or a G at position 50014440 of chromosome 10, a G or a C at position 35318088 of chromosome 20, an A or a G at position 45937922 of chromosome 14, an A or a G at position 45937935 of chromosome 14, and/or an A or a G at position 41422213 of chromosome 2 of the soybean genome, wherein the A at position 50014440 of chromosome 10, the G at position 35318088 of chromosome 20, the A at position 45937922 of chromosome 14, the A at position 45937935 of chromosome 14, and/or the A at position 41422213 of chromosome 2 is associated with high oleic acid, low linoleic acid, and/or low linolenic acid content.

In some embodiments, the genotyping comprises analyzing the at least one SNP or a haplotype using an oligonucleotide probe comprising a nucleic acid sequence of any one of SEQ ID NOs: 33, 34, 37, 38, 41, 42, 45, 46, 49, and 50; or a nucleic acid sequence complementary to a nucleic acid sequence of any one of SEQ ID NOs: 33, 34, 37, 38, 41, 42, 45, 46, 49, and 50. In some embodiments, the genotyping comprises analyzing the at least one SNP or a haplotype using a first primer and a second primer comprising any one pair of: (i) nucleic acid sequences of SEQ ID NOs: 35 and 36; (ii) nucleic acid sequences of SEQ ID NOs: 39 and 40; (iii) nucleic acid sequences of SEQ ID NOs: 43 and 44; (iv) nucleic acid sequences of SEQ ID NOs: 47 and 48; and (v) nucleic acid sequences of SEQ ID NOs: 51 and 52.

In some embodiments, the second population of progeny soybean plants or seeds comprises oil comprising: high palmitic acid and high oleic acid content; high palmitic acid and low linoleic acid content; high palmitic acid and low linolenic acid content; high palmitic acid, high oleic acid, and low linoleic acid content; high palmitic acid, high oleic acid, and low linolenic acid content; high palmitic acid, high oleic acid, low linoleic acid, low linolenic acid content; or high stearic acid, high oleic acid, low linoleic acid, and low linolenic acid content;
relative to a control population of soybean plants or seeds.

In some embodiments, the second population of progeny soybean plants or seeds comprises oil comprising high saturated fatty acid to unsaturated fatty acid composition relative to a control population of soybean plants or seeds. In some embodiments, the second population of progeny soybean plants or seeds comprises oil comprising high saturated plus monounsaturated fatty acids to polyunsaturated fatty acid composition relative to a control population of soybean plants or seeds. In some embodiments, the second population of progeny soybean plants or seeds comprises oil having a palmitic acid content of about 15% to about 30%, an oleic acid content of about 35% to about 80%, a linoleic acid content of about 5% to 25%, and/or a linolenic acid content of about 1% to about 5%.

In one aspect, the present disclosure provides a method of introgressing a quantitative trait locus (QTL) associated with high palmitic acid and/or high stearic acid content, the method comprising: (a) crossing a first soybean plant comprising a saturated QTL associated with high palmitic acid or high stearic acid content with a second soybean plant of a different genotype to produce one or more progeny plants or seeds; and (b) selecting a progeny plant or seed comprising an allele comprising a polymorphic locus associated with said saturated QTL, wherein the polymorphic locus is a chromosomal segment comprising a saturated marker within a genomic region 3567986-9738629 of chromosome 8 of a soybean genome. In some embodiments, the saturated QTL is Gm08:063500, Gm08:045000, Gm08:057400, Gm08:072100, Gm08:083900, Gm08:084300, Gm08:092100 or Gm08:126400. In some embodiments, said polymorphic locus comprises at least one single nucleotide polymorphisms (SNP), and the saturated marker comprises the at least one SNP. In some embodiments, the at least one SNP is a G or an A at position 4879302, a T or a C at position 3567986, a T or a C at position 4416970, an A or a T at position 5521970, an A or a T at position 6333332, a T or a C at position 6357981, a T or a C at position 6958927, and/or an A or a G at position 9738629 of chromosome 8 the soybean genome, wherein the G at position 4879302, the T at position 3567986, the T at position 4416970, the A at position 5521970, the A at position 6333332, the T at position 6357981, the T at position 6958927, or the A at position 9738629 of chromosome 8 of the soybean genome is associated with high palmitic acid content. In particular embodiments, the at least one SNP is a G or an A at position 4879302 of chromosome 8 and/or a T or an C at position 6357981 of chromosome 8 of a genome the soybean plants or seeds, wherein the G at position 4879302 of chromosome 8 or the T at position 6357981 of chromosome 8 is associated with high palmitic acid content.

In some embodiments, the method comprises: (a) crossing a first soybean plant comprising (i) the saturated QTL associated with high palmitic acid or high stearic acid content and (ii) an unsaturated QTL associated with high oleic acid, low linoleic acid, and/or low linolenic acid content with a second soybean plant of a different genotype to produce one or more progeny plants or seeds; and (b) selecting a progeny plant or seed comprising an allele comprising a polymorphic locus associated with said saturated QTL and a polymorphic locus associated with said unsaturated QTL, wherein the polymorphic locus linked to said unsaturated QTL is a chromosomal segment comprising an unsaturated marker within a genomic region 50013483-50015460 of chromosome 10, a genomic region 35315629-35319063 of chromosome 20, a genomic region 45935667-45939896 of chromosome 14, or a genomic region 41419655-41423881 of chromosome 2 of a soybean genome. In some embodiments, the unsaturated QTL associated with high oleic acid, high linoleic acid, and/or low linolenic acid content is Gm10:50014440, Gm20:35318088, Gm14:45937922, Gm14:45937935, and/or Gm02:41422213. In some embodiments, said polymorphic locus associated with said unsaturated QTL comprises at least one SNP, and said unsaturated marker comprises the at least one SNP. In some embodiments, the at least one SNP is an A or a G at position 50014440 of chromosome 10, a G or a C at position 35318088 of chromosome 20, an A or a G at position 45937922 of chromosome 14, an A or a G at position 45937935 of chromosome 14, and/or an A or a G at position 41422213 of chromosome 2 of the soybean genome, wherein the A at position 50014440 of chromosome 10, the G at position 35318088 of chromosome 20, the A at position 45937922 of chromosome 14, the A at position 45937935 of chromosome 14, and/or the A at position 41422213 of chromosome 2 is associated with high oleic acid, low linoleic acid, and/or low linolenic acid content.

In some embodiments of the methods provided herein, the soybean plant or seed is selected from the group consisting of Glycine max, Glycine soja, Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine stenophita, Glycine tabacina, and Glycine tomentella.

In one aspect, the present disclosure provides a population of soybean plants or seeds produced by the method provided herein, comprising high palmitic acid, high oleic acid, high linoleic acid, and/or low linolenic acid content relative to a control population of soybean plants or seeds. In some embodiments, the population comprises said saturated QTL associated with high palmitic acid or high stearic acid content, and/or an unsaturated QTL associated with high oleic acid, high linoleic acid, and/or low linolenic acid content at a greater frequency relative to a control population of soybean plants or seeds.

In one aspect, the present disclosure provides oil produced from a population of soybean plants or seeds produced by the method provided herein, or from the population of soybean plants or seeds provided herein, comprising: high palmitic acid content; high stearic acid content; high palmitic acid and high oleic acid content; high palmitic acid and low linoleic acid content; high palmitic acid and low linolenic acid content; high palmitic acid, high oleic acid, and low linoleic acid content; high palmitic acid, high oleic acid, and low linolenic acid content; high palmitic acid, high oleic acid, low linoleic acid, low linolenic acid content; or high stearic acid, high oleic acid, low linoleic acid, and low linolenic acid content, relative to oil produced from a control population of soybean plants or seeds. In some embodiments, the oil comprises high saturated fatty acid to unsaturated fatty acid composition relative to oil produced from a control population of soybean plants or seeds. In some embodiments, the oil comprises high saturated plus monounsaturated fatty acids to polyunsaturated fatty acid composition relative to oil produced from a control population of soybean plants or seeds. In some embodiments, the oil comprises at least about 4% increase in palmitic acid content or at least about 0.5% increase in stearic acid content relative to oil produced from a control population of soybean plants or seeds. In some embodiments, the oil comprises a palmitic acid content of about 15% to about 30% and/or a stearic acid content of about 2.5% to about 3.5%. In some embodiments, the oil comprises a palmitic acid content of about 15% to about 30%, an oleic acid content of about 35% to about 80%, a linoleic acid content of about 5% to 25%, and/or a linolenic acid content of about 1% to about 5%.

In one aspect, the present disclosure provides soybean oil comprising a palmitic acid content of about 15% to about 30% and an oleic acid content of about 35% to about 80%. In some embodiments, the soybean oil comprises a linoleic acid content of about 5% to about 25%. In some embodiments, the soybean oil comprises a linolenic acid content of about 1% to about 5%.

In some embodiments, the oil (e.g., soybean oil) provided herein comprises at least one saturated quantitative trait locus (QTL) associated with high palmitic acid or high stearic acid content and/or at least one unsaturated QTL associated with high oleic acid, low linoleic acid, and/or low linolenic acid content. In some embodiments, the at least one saturated QTL in the oil is Gm08:063500, Gm08:045000, Gm08:057400, Gm08:072100, Gm08:083900, Gm08:084300, Gm08:092100 and/or Gm08:126400, and the at least one unsaturated QTL in the oil is Gm10:50014440, Gm20:35318088, Gm14:45937922, Gm14:45937935, and/or Gm02:41422213.

In some embodiments, said at least one saturated QTL in the oil comprises at least one saturated SNP marker, and said at least one unsaturated QTL in the oil comprises at least one unsaturated SNP marker. In some embodiments, the at least one saturated SNP marker in the oil is a G or an A at position 4879302, a T or a C at position 3567986, a T or a C at position 4416970, an A or a T at position 5521970, an A or a T at position 6333332, a T or a C at position 6357981, a T or a C at position 6958927, or an A or a G at position 9738629 of chromosome 8; and wherein the at least one unsaturated SNP marker in the oil is an A or a G at position 50014440 of chromosome 10, a G or a C at position 35318088 of chromosome 20, an A or a G at position 45937922 of chromosome 14, an A or a G at position 45937935 of chromosome 14, and/or an A or a G at position 41422213 of chromosome 2 of the soybean genome, wherein the G at position 4879302, the T at position 3567986, the T at position 4416970, the A at position 5521970, the A at position 6333332, the T at position 6357981, the T at position 6958927, and/or the A at position 9738629 of chromosome 8 is associated with high palmitic acid content; and the A at position 50014440 of chromosome 10, the G at position 35318088 of chromosome 20, the A at position 45937922 of chromosome 14, the A at position 45937935 of chromosome 14, and/or the A at position 41422213 of chromosome 2 is associated with high oleic acid, low linoleic acid, and/or low linolenic acid content.

In one aspect, the present disclosure provides a nucleic acid molecule for detecting a molecular marker in a soybean genome or oil associated with high palmitic acid, high oleic acid, low linoleic acid, and/or low linolenic acid content, wherein the nucleic acid molecule comprises at least 15 nucleotides and has at least 90% sequence identity to a sequence of the same number of contiguous nucleotides of a sense or antisense DNA strand in a region comprising or adjacent to the molecular marker, wherein the molecular marker is located in a genomic region 3567986-9738629 of chromosome 8, a genomic region 50013483-50015460 of chromosome 10, a genomic region 35315629-35319063 of chromosome 20, a genomic region 45935667-45939896 of chromosome 14, or a genomic region 41419655-41423881 of chromosome 2 of a soybean genome. In some embodiments, the molecular marker is a single nucleotide polymorphism (SNP) marker, and wherein the SNP marker is selected from the group consisting of a G or an A at position 4879302, a T or a C at position 3567986, a T or a C at position 4416970, an A or a T at position 5521970, an A or a T at position 6333332, a T or a C at position 6357981, a T or a C at position 6958927, and an A or a G at position 9738629 of chromosome 8; an A or a G at position 50014440 of chromosome 10; a G or a C at position 35318088 of chromosome 20; an A or a G at position 45937922 of chromosome 14; an A or a G at position 45937935 of chromosome 14; and/or an A or a G at position 41422213 of chromosome 2 of the soybean genome. In some embodiments, said nucleic acid molecule comprises any one of SEQ ID NOs: 1, 2, 5, 6, 9, 10, 13, 14, 17, 18, 21, 22, 25, 26, 29, 30, 33, 34, 37, 38, 41, 42, 45, 46, 49, and 50. In some embodiments, the nucleic acid molecule provided herein further comprises a detectable label. In some embodiments, said detectable label is a fluorescent label or a radioactive label.

### DETAILED DESCRIPTION OF THE INVENTION

### I. References and Definitions

The present disclosure now will be described more fully hereinafter. The disclosure may be embodied in many different forms and should not be construed as limited to the aspects set forth herein; rather, these aspects are provided so that this disclosure will satisfy applicable legal requirements.

As used herein, “a,” “an,” or “the” can mean one or more than one. For example, “a” cell can mean a single cell or a multiplicity of cells. Further, the term “a plant” may include a plurality of plants.

As used herein, unless specifically indicated otherwise, the word “or” is used in the inclusive sense of “and/or” and not the exclusive sense of “either/or.”

The term “about” or “approximately” usually means within 5%, or more preferably within 1%, of a given value or range.

The terms “comprises”, “comprising”, “includes”, “including”, “having” and their conjugates mean “including but not limited to”.

Various embodiments of this disclosure may be presented in a range format. It should be noted that whenever a value or range of values of a parameter are recited, it is intended that values and ranges intermediate to the recited values are also part of this disclosure. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1-10 should be considered to have specifically disclosed subranges such as from 1 to 3, from 1 to 4, from 1 to 5, from 1 to 6, from 1 to 7, from 1 to 8, from 1 to 9, from 2 to 4, from 2 to 6, from 2 to 8, from 2 to 10, from 3 to 6, etc., as well as individual numbers within that range, for example, 1, 2, 3, 4, 5, 6, 7, 8, 9 and 10. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein, “quantitative trait locus” (QTL) or “quantitative trait loci” (QTLs) refer to a genetic domain that effects a phenotype that can be described in quantitative terms and can be assigned a “phenotypic value” which corresponds to a quantitative value for the phenotypic trait.

As used herein, “allele” refers to an alternative nucleic acid sequence at a particular locus. The length of an allele can be as small as one nucleotide base. For example, a first allele can occur on one chromosome, while a second allele occurs on a second homologous chromosome, e.g., as occurs for different chromosomes of a heterozygous individual, or between different homozygous or heterozygous individuals in a population.

As used herein, “locus” is a chromosome region or chromosomal region where a polymorphic nucleic acid, trait determinant, gene, or marker is located. A locus may represent a single nucleotide, a few nucleotides or a large number of nucleotides in a genomic region. The loci of this disclosure comprise one or more polymorphisms in a population; e.g., alternative alleles are present in some individuals. A “gene locus” is a specific chromosome location in the genome of a species where a specific gene can be found.

An allele of a QTL can, as used herein, can comprise multiple genes or other genetic factors even within a contiguous genomic region or linkage group, such as a haplotype. As used herein, an allele of a QTL can therefore encompasses more than one gene or other genetic factor where each individual gene or genetic component is also capable of exhibiting allelic variation and where each gene or genetic factor is also capable of eliciting a phenotypic effect on the quantitative trait in question. In an embodiment of the present invention the allele of a QTL comprises one or more genes or other genetic factors that are also capable of exhibiting allelic variation. The use of the term “an allele of a QTL” is thus not intended to exclude a QTL that comprises more than one gene or other genetic factor. Specifically, an “allele of a QTL” in the present in the invention can denote a haplotype within a haplotype window wherein a phenotype can be disease resistance. A haplotype window is a contiguous genomic region that can be defined, and tracked, with a set of one or more polymorphic markers wherein said polymorphisms indicate identity by descent. A haplotype within that window can be defined by the unique fingerprint of alleles at each marker. As used herein, an allele is one of several alternative forms of a gene occupying a given locus on a chromosome.

When all the alleles present at a given locus on a chromosome are the same, that plant is homozygous at that locus. If the alleles present at a given locus on a chromosome differ, that plant is heterozygous at that locus.

As used herein, a “haplotype” is the genotype of an individual at a plurality of genetic loci. Typically, the genetic loci described by a haplotype are physically and genetically linked, e.g., in the same chromosome interval. A haplotype can also refer to a combination of SNP alleles located within a single gene.

As used herein, “polymorphism” means the presence of one or more variations in a population. A polymorphism may manifest as a variation in the nucleotide sequence of a nucleic acid or as a variation in the amino acid sequence of a protein. Polymorphisms include the presence of one or more variations of a nucleic acid sequence or nucleic acid feature at one or more loci in a population of one or more individuals. The variation may comprise but is not limited to one or more nucleotide base changes, the insertion of one or more nucleotides or the deletion of one or more nucleotides. A polymorphism may arise from random processes in nucleic acid replication, through mutagenesis, as a result of mobile genomic elements, from copy number variation and during the process of meiosis, such as unequal crossing over, genome duplication and chromosome breaks and fusions. The variation can be commonly found or may exist at low frequency within a population, the former having greater utility in general plant breeding and the latter may be associated with rare but important phenotypic variation. Useful polymorphisms may include single nucleotide polymorphisms (SNPs), insertions or deletions in DNA sequence (Indels), simple sequence repeats of DNA sequence (SSRs), a restriction fragment length polymorphism, and a tag SNP. A genetic marker, a gene, a DNA-derived sequence, a RNA-derived sequence, a promoter, a 5′ untranslated region of a gene, a 3′ untranslated region of a gene, microRNA, siRNA, a tolerance locus, a satellite marker, a transgene, mRNA, ds mRNA, a transcriptional profile, and a methylation pattern may also comprise polymorphisms. In addition, the presence, absence, or variation in copy number of the preceding may comprise polymorphisms.

As used herein, “SNP” or “single nucleotide polymorphism” means a sequence variation that occurs when a single nucleotide (A, T, C, or G) in the genome sequence is altered or variable.

As used herein, “marker,” or “molecular marker,” or “marker locus” is a term used to denote a nucleic acid or amino acid sequence that is sufficiently unique to characterize a specific locus on the genome

As used herein, a centimorgan (“cM”) is a unit of measure of recombination frequency and genetic distance between two loci. One cM is equal to a 1% chance that a marker at one genetic locus will be separated from a marker at, a second locus due to crossing over in a single generation.

As used herein, “introgression” refers to the transmission of a desired allele of a genetic locus from one genetic background to another.

As used herein, “primer” refers to an oligonucleotide (synthetic or occurring naturally), which is capable of acting as a point of initiation of nucleic acid synthesis or replication along a complementary strand when placed under conditions in which synthesis of a complementary strand is catalyzed by a polymerase. Typically, primers are about 10 to 30 nucleotides in length, but longer or shorter sequences can be employed. Primers may be provided in double-stranded form, though the single-stranded form is more typically used. A primer can further contain a detectable label, for example a 5′ end label.

As used herein, “probe” refers to an oligonucleotide (synthetic or occurring naturally) that is complementary (though not necessarily fully complementary) to a polynucleotide of interest and forms a duplex structure by hybridization with at least one strand of the polynucleotide of interest. Typically, probes are oligonucleotides from 10 to 50 nucleotides in length, but longer or shorter sequences can be employed. A probe can further contain a detectable label, e.g., a fluorescent label or a radioactive label.

“Select”, “selected”, “desired”, or “preferred” seed oil composition, as used herein refers to a seed oil composition from a plant of the invention which has different levels of the fatty acids therein and is selected for particular purpose, relative to a seed oil from a control plant not having the genetic marker or the phenotype. A “low polyunsaturated” oil composition can contain about 30% or less polyunsaturated fatty acid content, e.g., about 5% to about 30% saturated fatty acid content (of total fatty acids). A “high saturated” oil composition can contain about 17.5% or more saturated fatty acid content, e.g., about 17.5% to about 35% (of total fatty acids) saturated fatty acid content. “Total oil level” or “total fatty acids” refers to the total aggregate amount of fatty acid without regard to the type of fatty acid.

As used herein with respect to a parameter such as oil content, the term “increased” or “high” refers to a detectable (e.g., about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) positive change in the parameter (e.g., oil content) from a comparison control, such as an established normal or reference level of the parameter, or an established standard control. For example, increased palmitic and/or oleic acid levels in a plant or plant part may indicate about 1%, 5%, 10%, 15%, 20%, 25%, 30%, 35%0, 40%, 45%, 50%, 55%, 60%, 65%, 70%, 75%, 80%, 85%, 90%, 95%, 96%, 97%, 98%, 99%, or 100% increase or positive change in a level of palmitic and/or oleic acid levels in a plant or plant part, as compared to that in a control plant or plant part.

As used herein with respect to a parameter such as oil content, the term “reduced”, “decreased”, or “low” refers to a detectable (e.g., about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%) negative change in the parameter (e.g., oil content) from a comparison control, such as an established normal or reference level of the parameter, or an established standard control. For example, reduced linolenic acid or linoleic acid level in a plant or plant part may indicate an at least about 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% decrease or negative change in a level of linolenic acid or linoleic acid in a plant or plant part, as compared to that in a control plant or plant part.

As used herein, the terms “phenotype,” or “phenotypic trait,” or “trait” refers to one or more detectable characteristics of a cell or organism which can be influenced by genotype. The phenotype can be observable to the naked eye, or by any other means of evaluation known in the art, e.g., microscopy, biochemical analysis, genomic analysis, an assay for a particular disease tolerance, etc. In some cases, a phenotype is directly controlled by a single gene or genetic locus, e.g., a “single gene trait.” In other cases, a phenotype is the result of several genes. In specific embodiments, the phenotype of soybean seeds is a high saturated fatty acid (e.g., palmitic and/or stearic acid), high monounsaturated fatty acid (e.g., oleic acid), and/or low polyunsaturated fatty acid (e.g., linoleic and linolenic acid) phenotype (i.e., a “HPHOLL” phenotype).

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, pulp, juice, kernels, ears, cobs, husks, stalks, roots, root tips, anthers, and the like. A plant cell is a biological cell of a plant, taken from a plant or derived through culture of a cell taken from a plant. Progeny, variants, and mutants of the regenerated plants are also included within the scope of the invention, provided that these parts comprise the introduced polynucleotides. Further provided is a processed plant product (e.g., extract) or byproduct that retains one or more polynucleotides disclosed herein. A progeny plant can be from any filial generation, e.g., F1, F2, F3, F4, F5, F6, F7, etc. A plant cell is a biological cell of a plant, taken from a plant or derived through culture from a cell taken from a plant.

As used herein, “cross” or “crossing” or “crossed” means to produce progeny via fertilization (e.g. cells, seeds or plants) and includes crosses between plants (sexual) and self-fertilization (selfing). Typically, a cross occurs after pollen is transferred from one flower to another, but those of ordinary skill in the art will understand that plant breeders can leverage their understanding of crossing, pollination, syngamy, and fecundation to circumvent certain steps of the plant life cycle and yet achieve equivalent outcomes, for example, a plant or cell of a soybean cultivar described herein. In certain embodiments, a user of this innovation can generate a plant of the claimed invention by removing a genome from its host gamete cell before syngamy and inserting it into the nucleus of another cell. While this variation avoids the unnecessary steps of pollination and syngamy and produces a cell that may not satisfy certain definitions of a zygote, the process falls within the definition of crossing as used herein when performed in conjunction with these teachings. In certain embodiments, the gametes are not different cell types (i.e., egg vs. sperm), but rather the same type and techniques are used to effect the combination of their genomes into a regenerable cell. Other embodiments of crossing include circumstances where the gametes originate from the same parent plant, i.e., a “self” or “self-fertilization”. While selfing a plant does not require the transfer of pollen from one plant to another, those of skill in the art will recognize that it nevertheless serves as an example of a cross. Thus, methods and compositions taught herein are not limited to certain techniques or steps that must be performed to create a plant or an offspring plant of the claimed invention, but rather include broadly any method that is substantially the same and/or results in compositions of the claimed invention.

As used herein, a “soybean plant” refers to a plant of species Glycine max (L) and includes all soybean varieties that can be bred with soybean, including wild soybean species such as Glycine soja. For example, “soybean plant” used herein includes Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine stenophita, Glycine tabacina, and Glycine tomentella.

A reference (e.g., control) sample of soybean plant or seed, e.g., a commodity soybean or seed, may have oil composition comprising palmitic acid, stearic acid, oleic acid, linoleic acid, and linolenic acid of about 10%, 4%, 18%, 55%, and 13%, respectively (Clemente & Cahoon 2009, Plant Physiol. 151:1030-1040).

A soybean plant, a soybean seed, or soybean oil with a “high palmitic acid” phenotype or content as used herein refers to a soybean plant, soybean seed, or soybean oil having a greater palmitic acid content as compared to a reference (e.g., control, commodity) sample of soybean plant, seed, or oil. A soybean plant, a soybean seed, or soybean oil with a “high palmitic acid” phenotype or content includes a soybean plant, a soybean seed, or soybean oil that has higher palmitic acid content, expressed as percent of total fatty acids, as compared to a reference (e.g., control, commodity) sample of soybean plant, seed, or oil, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “high palmitic acid” soybean plant, seed, or oil also includes a soybean plant, seed, or oil having a palmitic acid content of about 10% to about 50%, e.g., about 15-30%, 15-17.5%, 17.5-20%, 20-22.5%, 22.5-25%, 25-27.5%, 27.5-30%, 30-40%, 40-50%, 10% or more, 15% or more, 20% or more, 25% or more, 30% or more, 35% or more, 40% or more, 45% or more, or 50% or more of total fatty acids by weight.

A soybean plant, a soybean seed, or soybean oil with a “high stearic acid” phenotype or content as used herein refers to a soybean plant, soybean seed, or soybean oil having a greater stearic acid content as compared to a reference (e.g., control, commodity) sample of soybean plant, seed, or oil. A soybean plant, a soybean seed, or soybean oil with a “high stearic acid” phenotype or content includes a soybean plant, a soybean seed, or soybean oil that has higher stearic acid content, expressed as percent of total fatty acids, as compared to a reference (e.g., control, commodity) sample of soybean plant, seed, or oil, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “high stearic acid” soybean plant, seed, or oil also includes a soybean plant, seed, or oil having a stearic acid content of about 2% to about 10%, e.g., about 2.5-3.5%, 2-2.5%, 2.5-3%, 3-3.5%, 3.5-4%, 4-5%, 5-6%, 6-8%, 8-10%, 2% or more, 3% or more, 4% or more, 5% or more, 6% or more, 7% or more, 8% or more, 9% or more, or 10% or more of total fatty acids by weight.

A soybean plant, a soybean seed, or soybean oil with a “high saturated”, “high saturated fatty acid”, or “high palmitic and stearic acid”, or “high palmitic or stearic acid” phenotype or content as used herein refers to a soybean plant, soybean seed, or soybean oil having a greater saturated fatty acid (e.g., palmitic acid plus stearic acid) content as compared to a reference (e.g., control, commodity) sample of soybean plant, seed, or oil. A soybean plant, a soybean seed, or soybean oil with a “high saturated”, “high saturated fatty acid”, or “high palmitic and stearic acid”, or “high palmitic or stearic acid” phenotype or content includes a soybean plant, a soybean seed, or soybean oil that has higher stearic acid content, expressed as percent of total fatty acids, as compared to a reference (e.g., control, commodity) sample of soybean plant, seed, or oil, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “high saturated”, “high saturated fatty acid”, or “high palmitic and stearic acid”, or “high palmitic or stearic acid” soybean plant, seed, or oil also includes a soybean plant, seed, or oil having a saturated fatty acid content of about 10% to about 55%, e.g., about 17.5-35%, 10-15%, 15-17.5%, 17.5-20%, 20-22.5%, 22.5-25%, 25-27.5%, 27.5-30%, 30-40%, 40-50%, 10% or more, 15% or more, 20% or more, 25% or more, 30% or more, 35% or more, 40% or more, 45% or more, 50% or more, or 55% or more of total fatty acids by weight.

A soybean plant, a soybean seed, or soybean oil with a “high oleic acid” or “high monounsaturated fatty acid” phenotype or content as used herein refers to a soybean plant, soybean seed, or soybean oil having a greater oleic acid or monounsaturated fatty acid content as compared to a reference (e.g., control, commodity) sample of soybean plant, seed, or oil. A soybean plant, a soybean seed, or soybean oil with a “high oleic acid” or “high monounsaturated fatty acid” phenotype or content includes a soybean plant, a soybean seed, or soybean oil that has higher oleic acid or monounsaturated fatty acid content, expressed as percent of total fatty acids, as compared to a reference (e.g., control, commodity) sample of soybean plant, seed, or oil, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “high oleic acid” or “high monounsaturated fatty acid” soybean plant, seed, or oil also includes a soybean plant, seed, or oil having an oleic acid or monounsaturated fatty acid content of about 35% to about 80%, e.g., about 35-50%, 35-40%, 40-45%, 45-50%, 50-55%, 55-60%, 65-70%, 75-80%, 50-80%; 55-80%; 55-75%; 55-65%; 65-80%; 65-75%; 65-70%; 70-75%; or 75-80%; 30% or greater; 35% or greater; 40% or greater; 45% or greater; 50% or greater; 55% or greater; 60% or greater; 65% or greater; 70% or greater; 75% or greater; or 80% or greater of total fatty acids by weight.

A soybean plant, a soybean seed, or soybean oil with a “low linoleic acid” phenotype or content as used herein refers to a soybean plant, soybean seed, or soybean oil having a less linoleic acid content as compared to a reference (e.g., control, commodity) sample of soybean plant, seed, or oil. A soybean plant, a soybean seed, or soybean oil with a “low linoleic acid” phenotype or content includes a soybean plant, a soybean seed, or soybean oil that has lower linoleic acid content, expressed as percent of total fatty acids, as compared to a reference (e.g., control, commodity) sample of soybean plant, seed, or oil, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “low linoleic acid” soybean plant, seed, or oil also includes a soybean plant, seed, or oil having a linoleic acid content of about 5% to 25%, e.g., about 5-10%, 10-15%, 15-20%, 20-25%, 5% or less, 10% or less, 15% or less, 20% or less, 25% or less, or 30% or less of total fatty acids by weight.

A soybean plant, a soybean seed, or soybean oil with a “low linolenic acid” phenotype or content as used herein refers to a soybean plant, soybean seed, or soybean oil having a less linolenic acid content as compared to a reference (e.g., control, commodity) sample of soybean plant, seed, or oil. A soybean plant, a soybean seed, or soybean oil with a “low linolenic acid” phenotype or content includes a soybean plant, a soybean seed, or soybean oil that has lower linolenic acid content, expressed as percent of total fatty acids, as compared to a reference (e.g., control, commodity) sample of soybean plant, seed, or oil, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “low linolenic acid” soybean plant, seed, or oil also includes a soybean plant, seed, or oil having a linolenic acid content of about 1% to about 5%, e.g., about 1-2%, 2-3%, 3-4%, 4-5%, 1% or less, 2% or less, 3% or less, 4% or less, 5% or less, 6% or less, or 7% or less of total fatty acids by weight.

A soybean plant, a soybean seed, or soybean oil with a “low polyunsaturated fatty acid”, “low linoleic acid and linoleinic acid”, or “low linoleic acid or linolenic acid” phenotype or content as used herein refers to a soybean plant, soybean seed, or soybean oil having a less polyunsaturated fatty acid (e.g., linolenic plus linoleic acids) content as compared to a reference (e.g., control, commodity) sample of soybean plant, seed, or oil. A soybean plant, a soybean seed, or soybean oil with a “low polyunsaturated fatty acid”, “low linoleic acid and linoleinic acid”, or “low linoleic acid or linolenic acid” phenotype or content includes a soybean plant, a soybean seed, or soybean oil that has less polyunsaturated fatty acid (e.g., linoleic and/or linolenic acid) content, expressed as percent of total fatty acids, as compared to a reference (e.g., control, commodity) sample of soybean plant, seed, or oil, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “low polyunsaturated fatty acid”, “low linoleic acid and linoleinic acid”, or “low linoleic acid or linolenic acid” soybean plant, seed, or oil also includes a soybean plant, seed, or oil having a polyunsaturated fatty acid content of about 6% to 30%, e.g., about 5-6%, 6-10%, 10-15%, 15-20%, 20-25%, 5% or less, 6% or less, 10% or less, 15% or less, 20% or less, 25% or less, 30% or less, or 35% or less of total fatty acids by weight.

A soybean plant, a soybean seed, or soybean oil with a “high saturated fatty acid” (e.g., palmitic and/or stearic acid), “high monounsaturated fatty acid” (e.g., oleic acid), and/or “low polyunsaturated fatty acid” (e.g., linoleic and linolenic acid) phenotype, also referred to as a “HPHOLL” phenotype, as used herein refers to a soybean plant, soybean seed, or soybean oil having a greater saturated fatty acid content (e.g., a greater palmitic acid and/or stearic acid content), a greater monounsaturated fatty acid content (e.g., a greater oleic acid content), a less polyunsaturated fatty acid content (e.g., a less linoleic acid and/or linolenic acid content), a greater saturated to unsaturated fatty acid composition, or a greater saturated plus monounsaturated to polyunsaturated fatty acid composition, as compared to a reference sample of soybean plant or seed. An “HPHOLL” soybean plant, oil, or seed includes a plant, plant part, or plant product (e.g., oil) that has one or more characteristics of “high palmitic acid”, “high stearic acid”, “high palmitic plus stearic acid”, “high saturated fatty acid”, “high oleic acid”, “high monounsaturated fatty acid”, “low linoleic acid”, “low linolenic acid”, “low linolenic plus linoleic acid”, “low polyunsaturated fatty acid”, “high monounsaturated to polyunsaturated fatty acid”, and “high saturated plus monounsaturated to polyunsaturated fatty acid” content or composition provided herein. An HPHOLL soybean plant, seed, or oil also includes a soybean plant, seed, or oil that has a saturated fatty acid content of about 17.5% to about 35% (of total fatty acids) and a polyunsaturated fatty acid content of about 5% to 30% (of total fatty acids). An HPHOLL soybean plant, seed, or oil also includes a soybean plant, seed, or oil that has a palmitic acid content of at least 15%, 20%, 25%, or 30% (of total fatty acids) by weight; a stearic acid content of at least 2.5%, 3.0%, or 3.5% (of total fatty acids) by weight; an oleic acid content of at least 35%, 40%, 45%, 50%, 55%, 60%, 65%, 70%, 75%, or 80% (of total fatty acids) by weight; a linoleic acid content of 5% or less, 10% or less, 15% or less, 20% or less, 25% or less (of total fatty acids) by weight; a linolenic acid content of 1% or less, 2% or less, 3% or less, 4% or less, 5% or less (of total fatty acids) by weight; a saturated fatty acid content of at least 15%, 20%, 25%, 30%, or 35% (of total fatty acids) by weight; a saturated plus monounsaturated fatty acid content of at least 50%, 55%, 60%, 70%, 75%, 80%, 85%, 90%, 95%, 96%, 97%, 98%, or 99% (of total fatty acids) by weight. In particular embodiment an HPHOLL plant, seed, or oil comprises a palmitic acid content of about 15% to about 30%, a stearic acid content of about 2.5% to about 3.5%, an oleic acid content of about 35% to about 80%, a linoleic acid content of about 5% to 25%, and/or a linolenic acid content of about 1% to about 5% by weight, as normalized to total fatty acids (which represents 100%).

Amount or levels of total fatty acids and specific fatty acids can be measured by any methods for measuring fatty acid amount or levels, including gas chromatography-mass spectrometry (GC-MS) optionally with certain modifications (e.g., with or without initial lipid extraction, with or without isotope labeling of analytes). Fatty acid composition (e.g., percentage of specific fatty acids normalized to total fatty acids) can be calculated based on the amount or concentration of total fatty acids and specific fatty acids in the sample.

As used herein, a “population of plants,” “population of seeds”, “plant population”, or “seed population” means a set comprising any number, including one, of individuals, objects, or data from which samples are taken for evaluation, e.g., estimating quantitative trait locus (QTL). Most commonly, the terms relate to a breeding population of plants from which members are selected and crossed to produce progeny in a breeding program. A population of plants can include the progeny of a single breeding cross or a plurality of breeding crosses, and can be either actual plants or plant derived material, or in silico representations of the plants or seeds. The population members need not be identical to the population members selected for use in subsequent cycles of analyses or those ultimately selected to obtain final progeny plants or seeds. Often, a plant or seed population is derived from a single biparental cross, but may also derive from two or more crosses between the same or different parents. Although a population of plants or seeds may comprise any number of individuals, those of skill in the art will recognize that plant breeders commonly use population sizes ranging from one or two hundred individuals to several thousand, and that the highest performing 5-20% of a population is what is commonly selected to be used in subsequent crosses in order to improve the performance of subsequent generations of the population.

A reference population of soybean plants or seeds, e.g., a population of commodity soybean plants or seeds, may have oil composition comprising palmitic acid, stearic acid, oleic acid, linoleic acid, and linolenic acid of about 10%, 4%, 18%, 55%, and 13% in average, respectively (Clemente & Cahoon 2009, Plant Physiol. 151:1030-1040.

A population of soybean plants or seeds with a “high palmitic acid” phenotype or content as used herein refers to a soybean plant, soybean seed, or soybean oil having a greater palmitic acid content as compared to a reference (e.g., control, commodity) sample of soybean plant, seed, or oil. A population of soybean plants or seeds with a “high palmitic acid” phenotype or content includes a population of soybean plants or seeds that has higher palmitic acid content, expressed as percent of total fatty acids, as compared to a reference (e.g., control, commodity) population of soybean plants or seeds, with the difference (by subtraction) of at least about 1%, 2%, 3%, 4%, 5%, 6%, 7%. 8%, 9%, 10%, 15%, 20%, 25%, 30%, 35%0, 40%, 45%, 50%, 55%, 60%, 65%, 70%, 75%, 80%, 85%, 90%, 95%, or 99%. A “high palmitic acid” population of soybean plants or seeds also includes a population of soybean plants or seeds having a palmitic acid content of about 10% to about 50%, e.g., about 15-30%, 15-17.5%, 17.5-20%, 20-22.5%, 22.5-25%, 25-27.5%, 27.5-30%, 30-40%, 40-50%, 10% or more, 15% or more, 20% or more, 25% or more, 30% or more, 35% or more, 40% or more, 45% or more, or 50% or more of total fatty acids by weight.

A population of soybean plants or seeds with a “high stearic acid” phenotype or content as used herein refers to a soybean plant, soybean seed, or soybean oil having a greater stearic acid content as compared to a reference (e.g., control, commodity) population of soybean plants or seeds. A population of soybean plants or seeds with a “high stearic acid” phenotype or content includes a population of soybean plants or seeds that has higher stearic acid content, expressed as percent of total fatty acids, as compared to a reference (e.g., control, commodity) population of soybean plants or seeds, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “high stearic acid” population of soybean plants or seeds also includes a population of soybean plants or seeds having a stearic acid content of about 2% to about 10%, e.g., about 2.5-3.5%, 2-2.5%, 2.5-3%, 3-3.5%, 3.5-4%, 4-5%, 5-6%, 6-8%, 8-10%, 2% or more, 3% or more, 4% or more, 5% or more, 6% or more, 7% or more, 8% or more, 9% or more, or 10% or more of total fatty acids by weight.

A population of soybean plants or soybean seeds with a “high saturated”, “high saturated fatty acid”, or “high palmitic and stearic acid”, or “high palmitic or stearic acid” phenotype or content as used herein refers to a population of soybean plants or soybean seeds having a greater saturated fatty acid (e.g., palmitic acid plus stearic acid) content as compared to a reference (e.g., control, commodity) population of soybean plants or seeds. A population of soybean plants or soybean seeds with a “high saturated”, “high saturated fatty acid”, or “high palmitic and stearic acid”, or “high palmitic or stearic acid” phenotype or content includes a population of soybean plants or soybean seeds that has higher stearic acid content, expressed as percent of total fatty acids, as compared to a reference (e.g., control, commodity) population of soybean plants or seeds, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “high saturated”, “high saturated fatty acid”, or “high palmitic and stearic acid”, or “high palmitic or stearic acid” population of soybean plants or seeds also includes a population of soybean plants or seeds having a saturated fatty acid content of about 10% to about 55%, e.g., about 17.5-35%, 10-15%, 15-17.5%, 17.5-20%, 20-22.5%, 22.5-25%, 25-27.5%, 27.5-30%, 30-40%, 40-50%, 10% or more, 15% or more, 20% or more, 25% or more, 30% or more, 35% or more, 40% or more, 45% or more, 50% or more, or 55% or more of total fatty acids by weight.

A population of soybean plants or soybean seeds with a “high oleic acid” or “high monounsaturated fatty acid” phenotype or content as used herein refers to a soybean plant, soybean seed, or soybean oil having a greater oleic acid or monounsaturated fatty acid content as compared to a reference (e.g., control, commodity) population of soybean plants or seeds. A population of soybean plants or soybean seeds with a “high oleic acid” or “high monounsaturated fatty acid” phenotype or content includes a population of soybean plants or soybean seeds that has higher oleic acid or monounsaturated fatty acid content, expressed as percent of total fatty acids, as compared to a reference (e.g., control, commodity) population of soybean plants or seeds, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “high oleic acid” or “high monounsaturated fatty acid” population of soybean plants or seeds includes a population of soybean plants or seeds having an oleic acid or monounsaturated fatty acid content of about 35% to about 80%, e.g., about 35-50%, 35-40%, 40-45%, 45-50%, 50-55%, 55-60%, 65-70%, 75-80%, 50-80%; 55-80%; 55-75%; 55-65%; 65-80%; 65-75%; 65-70%; 70-75%; or 75-80%; 30% or greater; 35% or greater; 40% or greater; 45% or greater; 50% or greater; 55% or greater; 60% or greater; 65% or greater; 70% or greater; 75% or greater; or 80% or greater of total fatty acids by weight.

A population of soybean plants or soybean seeds with a “low linoleic acid” phenotype or content as used herein refers to a soybean plant, soybean seed, or soybean oil having a less linoleic acid content as compared to a reference (e.g., control, commodity) population of soybean plants or seeds. A population of soybean plants or soybean seeds with a “low linoleic acid” phenotype or content includes a population of soybean plants or soybean seeds that has lower linoleic acid content, expressed as percent of total fatty acids, as compared to a reference (e.g., control, commodity) population of soybean plants or seeds, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “low linoleic acid” population of soybean plants or seeds also includes a population of soybean plants or seeds having a linoleic acid content of about 5% to 25%, e.g., about 5-10%, 10-15%, 15-20%, 20-25%, 5% or less, 10% or less, 15% or less, 20% or less, 25% or less, or 30% or less of total fatty acids by weight.

A population of soybean plants or soybean seeds with a “low linolenic acid” phenotype or content as used herein refers to a soybean plant, soybean seed, or soybean oil having a less linolenic acid content as compared to a reference (e.g., control, commodity) population of soybean plants or seeds. A population of soybean plants or soybean seeds with a “low linolenic acid” phenotype or content includes a population of soybean plants or soybean seeds that has lower linolenic acid content, expressed as percent of total fatty acids, as compared to a reference (e.g., control, commodity) population of soybean plants or seeds, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “low linolenic acid” population of soybean plants or seeds also includes a population of soybean plants or seeds having a linolenic acid content of about 1% to about 5%, e.g., about 1-2%, 2-3%, 3-4%, 4-5%, 1% or less, 2% or less, 3% or less, 4% or less, 5% or less, 6% or less, or 7% or less of total fatty acids by weight.

A population of soybean plants or soybean seeds with a “low polyunsaturated fatty acid”, “low linoleic acid and linoleinic acid”, or “low linoleic acid or linolenic acid” phenotype or content as used herein refers to a soybean plant, soybean seed, or soybean oil having a less polyunsaturated fatty acid (e.g., linolenic plus linoleic acids) content as compared to a reference (e.g., control, commodity) population of soybean plants or seeds. A population of soybean plants or soybean seeds with a “low polyunsaturated fatty acid”, “low linoleic acid and linoleinic acid”, or “low linoleic acid or linolenic acid” phenotype or content includes a population of soybean plants or soybean seeds that has less polyunsaturated fatty acid (e.g., linoleic and/or linolenic acid) content, expressed as percent of total fatty acids, as compared to a reference (e.g., control, commodity) population of soybean plants or seeds, with the difference (by subtraction) of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. A “low polyunsaturated fatty acid”, “low linoleic acid and linoleinic acid”, or “low linoleic acid or linolenic acid” population of soybean plants or seeds also includes a population of soybean plants or seeds having a polyunsaturated fatty acid content of about 6% to 30%, e.g., about 5-6%, 6-10%, 10-15%, 15-20%, 20-25%, 5% or less, 6% or less, 10% or less, 15% or less, 20% or less, 25% or less, 30% or less, or 35% or less of total fatty acids by weight.

A population of soybean plants or soybean seeds with a “high saturated fatty acid” (e.g., palmitic and/or stearic acid), “high monounsaturated fatty acid” (e.g., oleic acid), and/or “low polyunsaturated fatty acid” (e.g., linoleic and linolenic acid) phenotype, also referred to as a “HPHOLL” phenotype, as used herein refers to a soybean plant, soybean seed, or soybean oil having a greater saturated fatty acid content (e.g., a greater palmitic acid and/or stearic acid content), a greater monounsaturated fatty acid content (e.g., a greater oleic acid content), a less polyunsaturated fatty acid content (e.g., a less linoleic acid and/or linolenic acid content), a greater saturated to unsaturated fatty acid composition, or a greater saturated plus monounsaturated to polyunsaturated fatty acid composition, as compared to a reference sample of soybean plant or seed. An “HPHOLL” soybean plant, oil, or seed includes a plant, plant part, or plant product (e.g., oil) that has one or more characteristics of “high palmitic acid”, “high stearic acid”, “high palmitic plus stearic acid”, “high saturated fatty acid”, “high oleic acid”, “high monounsaturated fatty acid”, “low linoleic acid”, “low linolenic acid”, “low linolenic plus linoleic acid”, “low polyunsaturated fatty acid”, “high monounsaturated to polyunsaturated fatty acid”, and “high saturated plus monounsaturated to polyunsaturated fatty acid” content or composition provided herein. An HPHOLL population of soybean plants or seeds also includes a population of soybean plants or seeds that has a saturated fatty acid content of about 17.5% to about 35% (of total fatty acids) and a polyunsaturated fatty acid content of about 5% to 30% (of total fatty acids). An HPHOLL population of soybean plants or seeds also includes a population of soybean plants or seeds that has a palmitic acid content of at least 15%, 20%, 25%, or 30% (of total fatty acids) by weight; a stearic acid content of at least 2.5%, 3.0%, or 3.5% (of total fatty acids) by weight; an oleic acid content of at least 35%, 40%, 45%, 50%, 55%, 60%, 65%, 70%, 75%, or 80% (of total fatty acids) by weight; a linoleic acid content of 5% or less, 10% or less, 15% or less, 20% or less, 25% or less (of total fatty acids) by weight; a linolenic acid content of 1% or less, 2% or less, 3% or less, 4% or less, 5% or less (of total fatty acids) by weight; a saturated fatty acid content of at least 15%, 20%, 25%, 30%, or 35% (of total fatty acids) by weight; a saturated plus monounsaturated fatty acid content of at least 50%, 55%, 60%, 70%, 75%, 80%, 85%, 90%, 95%, 96%, 97%, 98%, or 99% (of total fatty acids) by weight. In particular embodiment an HPHOLL population of plants or seeds comprises a palmitic acid content of about 15% to about 30%, a stearic acid content of about 2.5% to about 3.5%, an oleic acid content of about 35% to about 80%, a linoleic acid content of about 5% to 25%, and/or a linolenic acid content of about 1% to about 5% by weight, as normalized to total fatty acids (which represents 100%).

As used herein, the term “crop performance” is used synonymously with “plant performance” and refers to of how well a plant grows under a set of environmental conditions and cultivation practices. Crop performance can be measured by any metric a user associates with a crop's productivity (e.g., yield), appearance and/or robustness (e.g., color, morphology, height, biomass, maturation rate, etc.), product quality (e.g., oil composition, oil content, oil quality, fiber lint percent, fiber quality, seed protein content, etc.), cost of goods sold (e.g., the cost of creating a seed, plant, or plant product in a commercial, research, or industrial setting) and/or a plant's tolerance to disease (e.g., a response associated with deliberate or spontaneous infection by a pathogen) and/or environmental stress (e.g., drought, flooding, low nitrogen or other soil nutrients, wind, hail, temperature, day length, etc.). Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g., the volume or weight of seed or other plant product measured in liters or grams) or some other metric assigned to some aspect of a plant that can be represented on a scale (e.g., assigning a 1-10 value to a plant based on its disease tolerance).

As used herein, “yield penalty” refers to a reduction of seed yield in a line correlated with or caused by the presence of an HPHOLL allele as compared to a line that does not contain that HPHOLL allele. In some embodiments, a yield penalty can be a partial yield penalty, such as a reduction of yield by about 0.1%, 0.5%, 1.0%, 1.5%, 2.0%, 2.5%, 3.0%, 3.5%, 4.0%, 4.5%, or about 5.0%, 6%, 7%, 8%, 9%, or about a 10% reduction in yield when compared to a soybean variety that does not contain the HPHOLL allele. In specific embodiments, the yield penalty is about a 0-5%, 0.5-4.5%, 0.5-4%, 1-5%, 1-4%, 2-5%, 2-4%, 0.5-10%, 0.5-8%, 1-10%, 2-10%, 3-10%, 4-10%, 5-10%, 6-10%, 7-10%, or about an 8-10% reduction in yield when compared to a soybean variety that does not contain the HPHOLL allele. Yield can be measured and expressed by any means known in the art. In specific embodiments, yield is measured by seed weight or volume in a given harvest area.

As used herein, “selecting” or “selection” in the context of marker-assisted selection or breeding refer to the act of picking or choosing desired individuals, normally from a population, based on certain pre-determined criteria.

As used herein the term “polynucleotide” refers to a single or double stranded nucleic acid sequence which is isolated and provided in the form of an RNA sequence (e.g., an mRNA sequence), a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein, “sequence identity,” “identity,” “percent identity,” “percentage similarity,” “sequence similarity” and the like refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol.266:131-141; Altschul et al. (1997), Nucleic Acids Res. 25:3389-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. (Proc Natl Acad Sci 89:10915-9 (1992)). Identity (e.g., percent homology) can be determined using any homology comparison software, including for example, the BlastN software of the National Center of Biotechnology Information (NCBI) such as by using default parameters.

According to some embodiments, the identity is a global identity, i.e., an identity over the entire amino acid or nucleic acid sequences of the invention and not over portions thereof. When reference is made to particular nucleic acid sequences, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

In certain embodiments, a user can combine the teachings herein with high-density molecular marker profiles spanning substantially the entire genome of a plant to estimate the value of selecting certain candidates in a breeding program in a process commonly known as genome selection.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

### II. Methods of Producing a High Saturated, High Monounsaturated, and/or Low Polyunsaturated Fatty Acid (HPHOLL) Soybean Plants or Seeds

In an aspect, the present disclosure provides a method of creating a population of high saturated fatty acid (i.e., with a high palmitic acid content) or HPHOLL [i.e., with a high saturated fatty acid (e.g., palmitic and/or stearic acid), high monounsaturated fatty acid (e.g., oleic acid), and/or low polyunsaturated fatty acid (e.g., linoleic and linolenic acid) content] soybean plants or seeds.

In certain aspects, the method provided herein uses a saturated marker, and comprises the steps of (a) genotyping a first population of soybean plants or seeds for the presence of at least one saturated marker associated with high palmitic acid and/or high stearic acid content, wherein the at least one saturated marker is within 20 centimorgans of at least one saturated quantitative trait locus (QTL) associated with high palmitic acid and/or high stearic acid content located within a genomic region 3567986-9738629 of chromosome 8 of a soybean genome; (b) selecting from the first population one or more soybean plants or seeds comprising one or more alleles comprising said at least one saturated marker associated with high palmitic acid and/or high stearic acid content; (c) producing a second population of progeny soybean plants or seeds from the one or more soybean plants or soybean seeds selected from the first population; and (d) selecting from the second population one or more progeny soybean plants or seeds comprising one or more (e.g., two) alleles comprising said at least one saturated marker associated with high palmitic acid and/or high stearic acid content, such that the selected one or more progeny soybean plants or seeds comprise high palmitic acid and/or high stearic acid content relative to a control plant or seed. A “saturated” marker or a “saturated” QTL as used herein refers to a marker or a QTL associated with high saturated fatty acid content (e.g., high palmitic acid and/or high stearic acid content) in a plant, plant seed, or plant oil relative to a control plant, plant seed, or plant oil.

In certain aspects, the method provided herein further uses an unsaturated marker, and comprises the steps of (a) genotyping the first population of soybean plants or seeds for the presence of (i) at least one saturated marker provided herein and (ii) at least one unsaturated marker associated with high oleic acid, low linoleic acid, and/or low linolenic acid content, wherein the at least one unsaturated marker is within 20 centimorgans of at least one unsaturated QTL associated with high oleic acid, low linoleic acid, and/or low linolenic acid content; (b) selecting from the first population one or more soybean plants or seeds comprising one or more alleles comprising (i) at least one saturated marker associated with high palmitic acid and/or high stearic acid content and (ii) at least one unsaturated marker associated with high oleic acid, low linoleic acid, and/or low linolenic acid content; (c) producing a second population of progeny soybean plants or seeds from the one or more soybean plants or soybean seeds selected from the first population; and (d) selecting from the second population one or more progeny soybean plants or seeds comprising one or more (e.g., two) alleles comprising (i) said at least one saturated marker associated with high palmitic acid and/or high stearic acid content and (ii) said at least one unsaturated marker associated with high oleic acid, low linoleic acid, and/or low linolenic acid content, such that the selected one or more progeny soybean plants or seeds comprise high palmitic acid, high oleic acid, low linoleic acid, and/or low linolenic acid (HPHOLL) content relative to a control plant or seed. An “unsaturated” marker or a “unsaturated” QTL as used herein refers to a marker or a QTL associated with high monounsaturated fatty acid content (e.g., high oleic acid content) and/or low polyunsaturated fatty acid content (e.g., low linoleic acid content and/or low linolenic acid content) in a plant, plant seed, or plant oil relative to a control plant, plant seed, or plant oil.

In some embodiments, the saturated QTL associated with high palmitic acid or high stearic acid content is Gm08:063500 (also referred to as PA_QTL08), Gm08:045000 (also referred to as PA_QTL08.1), Gm08:057400 (also referred to as PA_QTL08.2), Gm08:072100 (also referred to as PA_QTL08.3), Gm08:083900 (also referred to as PA_QTL08.4), Gm08:084300 (also referred to as PA_QTL08.5), Gm08:092100 (also referred to as PA_QTL08.6), or Gm08:126400 (also referred to as PA_QTL08.8). In some embodiments, the unsaturated QTL associated with high oleic acid, low linoleic acid, and/or low linolenic acid content is Gm10:50014440 (also referred to as FAD2_1A), Gm20:35318088 (also referred to as FAD2_1B), Gm14:45937922 (also referred to as FAD3A_SP or FAD3A_MO_SP), Gm14:45937935 (also referred to as FAD3A NS), or Gm02:41422213 (also referred to as FAD3B_MO). In some embodiments, the unsaturated marker associated with high oleic acid, low linoleic acid, and/or low linolenic acid content is located in Glyma.10G278000, Glyma.20G111000, Glyma.14G194300, or Glyma.02G227200 of the soybean plants or seeds.

QTLs (i.e., saturated QTLs, unsaturated QTLs) that exhibit significant co-segregation with high saturated, high monounsaturated, and/or low polyunsaturated fatty acid (i.e., HPHOLL) phenotype are provided herein. In specific embodiments, plants or seeds comprising the saturated and/or unsaturated QTLs further comprise one or more allele associated with an HPHOLL content. In some embodiments, the one or more allele associated with an HPHOLL content is within 20 centimorgans or within 10 centimorgans from one or more saturated or unsaturated QTLs. Saturated or unsaturated QTLs can be tracked during plant breeding or introgressed into a desired genetic background in order to provide plants exhibiting a high saturated, high monounsaturated, and/or low polyunsaturated fatty acid content and, in specific embodiments, one or more other beneficial traits. In an aspect, this disclosure identifies QTL intervals that are associated with HPHOLL in different soybean varieties described herein.

Saturated or unsaturated (e.g., HPHOLL) markers of the present disclosure include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers. In a diploid organism such as soybeans, a marker genotype typically comprises two marker alleles at each locus. The marker allelic composition of each locus can be either homozygous or heterozygous. Homozygosity is a condition where both alleles at a locus are characterized by the same nucleotide sequence. Heterozygosity refers to different conditions of the gene at a locus.

Saturated or unsaturated markers can be simple sequence repeat markers (SSR, also referred to as simple sequence length polymorphisms (SSLPs)), amplified fragment length polymorphism (AFLP) markers, restriction fragment length polymorphism (RFLP) markers, RAPD markers, phenotypic markers, single nucleotide polymorphisms (SNPs), isozyme markers, deletion markers, microarray transcription profiles that are genetically linked to or correlated with alleles of a QTL of the present invention (Walton, Seed World 22-29 (July, 1993), Burow et al., Molecular Dissection of Complex Traits, 13-29, ed. Paterson, CRC Press, New York (1988)). Methods to isolate and identify such markers are known in the art. For example, locus-specific SSR markers can be obtained by screening a genomic library for microsatellite repeats, sequencing of “positive” clones, designing primers which flank the repeats, and amplifying genomic DNA with these primers. The size of the resulting amplification products can vary by integral numbers of the basic repeat unit. Polymorphisms comprising as little as a single nucleotide change can be assayed in a number of ways. For example, detection can be made by electrophoretic techniques including a single strand conformational polymorphism (Orita et al., 1989), denaturing gradient gel electrophoresis (Myers et al., 1985), cleavage fragment length polymorphisms (Life Technologies, Inc., Gathersberg, Md. 20877), or direct sequencing of amplified products. Once the polymorphic sequence difference is known, rapid assays can be designed for progeny testing, typically involving some version of PCR amplification of specific alleles (PASA, Sommer, et al., 1992), or PCR amplification of multiple specific alleles (PAMSA, Dutton and Sommer, 1991). PCR products can be radiolabeled, separated on denaturing polyacrylamide gels, and detected by autoradiography. Fragments with size differences >4 bp can also be resolved on agarose gels, thus avoiding radioactivity.

A single nucleotide polymorphisms (SNP) occurs at a single nucleotide. SNPs are more stable than other classes of polymorphisms. Their spontaneous mutation rate is approximately 10-9 (Kornberg, DNA Replication, W. H. Freeman & Co., San Francisco (1980)). As SNPs result from sequence variation, new polymorphisms can be identified by sequencing random genomic or cDNA molecules. SNPs can also result from deletions, point mutations and insertions. That said, SNPs are also advantageous as markers since they are often diagnostic of “identity by descent” because they rarely arise from independent origins. Any single base alteration, whatever the cause, can be a SNP. SNPs occur at a greater frequency than other classes of polymorphisms and can be more readily identified. In the present disclosure, a SNP can represent a single indel event, which may consist of one or more base pairs, or a single nucleotide polymorphism.

In some embodiments, the saturated and/or unsaturated QTL comprises at least one SNP, and the at least one saturated and/or unsaturated marker comprises an allele of the at least one SNP. In some embodiments, the SNP contained in the saturated QTL is a G or an A at position 4879302 of chromosome 8, a T or a C at position 3567986 of chromosome 8, a T or a C at position 4416970 of chromosome 8, an A or a T at position 5521970 of chromosome 8, an A or a T at position 6333332 of chromosome 8, a T or a C at position 6357981 of chromosome 8, a T or a C at position 6958927 of chromosome 8, and/or an A or a G at position 9738629 of chromosome 8 the soybean genome. The G at position 4879302, the T at position 3567986, the T at position 4416970, the A at position 5521970, the A at position 6333332, the T at position 6357981, the T at position 6958927, or the A at position 9738629 of chromosome 8 of the soybean genome can be a marker associated with high palmitic acid content. In specific embodiments, the SNP contained in the saturated QTL is a G or an A at position 4879302 of chromosome 8 and/or a T or a C at position 6357981 of chromosome 8 of the soybean genome, and the G at position 4879302 of chromosome 8 and the T at position 6357981 of chromosome 8 are associated with high palmitic acid content. In some embodiments, the SNP contained in the unsaturated QTL is an A or a G at position 50014440 of chromosome 10, a G or a C at position 35318088 of chromosome 20, an A or a G at position 45937922 of chromosome 14, an A or a G at position 45937935 of chromosome 14, and/or an A or a G at position 41422213 of chromosome 2 of the soybean genome. The A at position 50014440 of chromosome 10, the G at position 35318088 of chromosome 20, the A at position 45937922 of chromosome 14, the A at position 45937935 of chromosome 14, or the A at position 41422213 of chromosome 2 can be associated with high oleic acid, low linoleic acid, and/or low linolenic acid content.

In some embodiments, the saturated or unsaturated QTL comprises a deletion marker. As used herein, a “deletion marker” refers to a deletion of a nucleotide region in the genome of plants or plant parts associated with (e.g., preventing) an HPHOLL phenotype. Plants or plant parts having genomes having the deletion marker can exhibit a higher saturated, higher monounsaturated, and/or lower polyunsaturated fatty acid content by weight as compared to the plants and plant parts lacking the deletion marker. The deleted nucleotide region of a deletion marker can be a deletion of any number of consecutive nucleotides that is associated with an HPHOLL phenotype. For example, the deletion can be 2-500 bp, 5-250 bp, 10-200 bp, 20-180 bp, 40-160 bp, 50-140 bp, 60-120 bp, 70-100 bp, 80-100 bp, 85-95 bp, or about 2 bp, 5 bp, 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 100 bp, 105 bp, 110 bp, 120 bp, 130 bp, 140 bp, 150 bp, 160 bp, 170 bp, 180 bp, 200 bp, 225 bp, 250 bp, 275 bp, 300 bp, 350 bp, 400 bp, 450 bp, or about 500 bp. In specific embodiments, the deletion maker can be wholly or at least partially within a gene. The deletion marker can be wholly or at least partially within an exon or intron of the gene. That is, the deletion marker can be a deletion of a nucleotide sequence entirely within a gene or spanning the 5′ end of the gene or the 3′ of the gene. In some embodiments, the deletion marker eliminates the start codon of a gene. The deletion marker can also account for removal of a signal peptide of a gene. In some embodiments, the deletion marker eliminates both the start codon and the signal peptide of a gene. The gene can be any gene in the genome.

The saturated and/or unsaturated QTLs disclosed herein can be an expression QTL (eQTL). As used herein an eQTL refers to a QTL that is associated with differential expression of a gene. In specific embodiments, when a QTL is present in the genome, a gene associated with the eQTL is has reduced expression. For example, the presence of an eQTL can eliminate or substantially elimination expression of a gene.

In some embodiments, selecting from the first population one or more soybean plants or seeds is based on detection of the presence of an SNP or a haplotype associated with an HPHOLL phenotype. A “haplotype” as used herein refers to a plurality of SNPs. An HPHOLL haplotype can comprise HPHOLL alleles of two or more polymorphic loci (e.g., saturated and/or unsaturated loci) described herein. In some embodiments, the genotyping according to the methods provided herein comprises analyzing the at least one SNP or the haplotype using an oligonucleotide probe comprising at least 15 nucleotides, wherein the oligonucleotide probe has at least 90% sequence identity to a sequence of the same number of contiguous nucleotides of a sense or antisense DNA strand in a region comprising or adjacent to the at least one SNP in the soybean genome. For example, the oligonucleotide probe can comprise a nucleic acid sequence having at least 90% identity to a nucleic acid sequence of any one of SEQ ID NOs: 1, 2, 5, 6, 9, 10, 13, 14, 17, 18, 21, 22, 25, 26, 29, and 30 or a nucleic acid sequence of any one of SEQ ID NOs: 1, 2, 5, 6, 9, 10, 13, 14, 17, 18, 21, 22, 25, 26, 29, and 30 for detection of a saturated SNP marker. The oligonucleotide probe can comprise a nucleic acid sequence having at least 90% identity to a nucleic acid sequence of any one of SEQ ID NOs: 33, 34, 37, 38, 41, 42, 45, 46, 49, and 50 or a nucleic acid sequence of any one of SEQ ID NOs: 33, 34, 37, 38, 41, 42, 45, 46, 49, and 50 for detection of an unsaturated SNP marker.

Additionally or alternatively, genotyping can comprise analyzing the at least one SNP or the haplotype using a first primer and a second primer each comprising at least 15 nucleotides, using PCR or quantitative PCR. The first primer can have at least 90% sequence identity to a sequence of the same number of contiguous nucleotides of a sense DNA strand of a region comprising or adjacent to the at least one SNP, and the second primer can have at least 90% sequence identity to a sequence of the same number of contiguous nucleotides of an antisense DNA strand of the region comprising or adjacent to the at least one SNP. For example, the first and second primers can comprise (i) nucleic acid sequences having at least 90% identity to nucleic acid sequences of SEQ ID NOs: 3 and 4, or nucleic acid sequences of SEQ ID NOs: 3 and 4; or (ii) nucleic acid sequences having at least 90% identity to nucleic acid sequences of SEQ ID NOs: 7 and 8, or nucleic acid sequences of SEQ ID NOs: 7 and 8; (iii) nucleic acid sequences having at least 90% identity to nucleic acid sequences of SEQ ID NOs: 11 and 12, or nucleic acid sequences of SEQ ID NOs: 11 and 12; (iv) nucleic acid sequences having at least 90% identity to nucleic acid sequences of SEQ ID NOs: 15 and 15, or nucleic acid sequences of SEQ ID NOs: 15 and 16; (v) nucleic acid sequences having at least 90% identity to nucleic acid sequences of SEQ ID NOs: 19 and 20, or nucleic acid sequences of SEQ ID NOs: 19 and 20; (vi) nucleic acid sequences having at least 90% identity to nucleic acid sequences of SEQ ID NOs: 23 and 24, or nucleic acid sequences of SEQ ID NOs: 23 and 24; (vii) nucleic acid sequences having at least 90% identity to nucleic acid sequences of SEQ ID NOs: 27 and 28, or nucleic acid sequences of SEQ ID NOs: 27 and 28; and (viii) nucleic acid sequences having at least 90% identity to nucleic acid sequences of SEQ ID NOs: 31 and 32, or nucleic acid sequences of SEQ ID NOs: 31 and 32, for detection of a saturated SNP marker. The first and second primers can comprise (i) nucleic acid sequences having at least 90% identity to nucleic acid sequences of SEQ ID NOs: 35 and 36; 39 and 40; or 43 and 44; 47 and 48; or 51 and 52, or (ii) a nucleic acid sequence of SEQ ID NOs: 35 and 36; 39 and 40; or 43 and 44; 47 and 48; or 51 and 52 for detection of an unsaturated SNP.

Additionally or alternatively, genotyping can comprise assaying a deletion marker. Any method known in the art can be used to identify a region of the genome that is missing a given position, including but not limited to PCR, RFLP, probe-based detection methods, and sequencing methods, among others.

In specific embodiments, the presence of saturated molecular markers in a plant, plant part, plant seed, or plant oil is associated with a higher saturated fatty acid content than corresponding plants, plant parts, plant seeds, or plant oil without the saturated molecular markers. The higher saturated fatty acid content in plants, plant parts, plant seeds, or plant oil having at least one saturated molecular marker (e.g., SNP or deletion marker) disclosed herein can be at least about 1%, 2%, 3%, or 4% greater, expressed as difference in % of total fatty acids in dry weight, compared to corresponding plants, plant parts, plant seeds, or plant oil without the saturated molecular marker. In certain embodiments, plants, plant parts, plant seeds, or plant oil having at least one saturated molecular marker (e.g., SNP or deletion marker) disclosed herein comprise at least about 4 percent increase in palmitic acid content or about 0.5 percent increase in stearic acid content, expressed as difference in % of total fatty acids in dry weight, relative to a control plant, plant part, plant seed, or plant oil without the saturated molecular marker. In some embodiments, the plants, plant parts, plant seeds, or plant oil having at least one saturated molecular marker disclosed herein can have higher saturated fatty acid content, expressed as percent of total fatty acids, as compared to a control plant, plant part, plant seed, or plant oil without the saturated molecular marker, expressed as percent of total fatty acids, and the difference (by subtraction) can be at least about 1%, 5%0, 10%, 15%, 20%, 25%, 30%, 35%, 40%, 45%, 50%, 55%, 60%, 65%, 70%, 75%, 80%, 85%, 90%, 95%, or 99%. In some embodiments, the plants, plant parts, plant seeds, or plant oil having at least one saturated molecular marker disclosed herein can comprise a palmitic acid content of about 15% to about 30% (e.g., about 15-17.5%, 17.5-20%, 20-22.5%, 22.5-25%, 25-27.5%, 27.5-30%) and/or a stearic acid content of about 2.5% to about 3.5% (e.g., about 2.5-3%, 3-3.5%).

In certain embodiments, the presence of unsaturated molecular markers in a plant, plant part, plant seed, or plant oil is associated with a higher monounsaturated fatty acid and/or a lower polyunsaturated fatty acid content compared to corresponding plants, plant parts, plant seeds, or plant oil without the unsaturated molecular marker. In some embodiments, the plants, plant parts, plant seeds, or plant oil having at least one unsaturated molecular marker (e.g., SNP or deletion marker) disclosed herein can have higher monounsaturated fatty acid content, expressed as percent of total fatty acids, as compared to that of a control plant, plant part, plant seed, or plant oil without the unsaturated molecular marker, expressed as percent of total fatty acids, and the difference (by subtraction) can be at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. In some embodiments, the plants, plant parts, plant seeds, or plant oil having at least one unsaturated molecular marker (e.g., SNP or deletion marker) disclosed herein can have lower polyunsaturated fatty acid content, expressed as percent of total fatty acids, as compared to that of a control plant, plant part, plant seed, or plant oil without the unsaturated molecular marker, expressed as percent of total fatty acids, and the difference (by subtraction) can be at least about 1%, 2%, 3%, 4%, 5%, 6%, 7%, 8%, 9%, 10%, 15%, 20%, 25%, 30%, 35%, 40%4, 45%, 50%, 55%, 60%, 65%, 70%, 75%, 80%, 85%, 90%, 95%, or 99%.

In certain embodiments, the presence of saturated and unsaturated molecular markers (e.g., SNP or deletion marker) in a plant, plant part, plant seed, or plant oil is associated with HPHOLL characteristics, including a greater saturated fatty acid content (e.g., a greater palmitic acid and/or stearic acid content), a greater monounsaturated fatty acid content (e.g., a greater oleic acid content), a less polyunsaturated fatty acid content (e.g., a less linoleic acid and/or linolenic acid content), a greater saturated to unsaturated fatty acid composition, a greater saturated plus monounsaturated to polyunsaturated fatty acid composition, and combination or variation of any thereof, as compared to a corresponding plant, plant part, plant seed, or plant oil without the saturated molecular marker(s) or the unsaturated molecular marker(s). For example, plants, plant parts, plant seeds, or plant oil having at least one saturated molecular marker and at least one unsaturated molecular marker can comprise high palmitic acid and high oleic acid content; high palmitic acid and low linoleic acid content; high palmitic acid and low linolenic acid content; high palmitic acid, high oleic acid, and low linoleic acid content; high palmitic acid, high oleic acid, and low linolenic acid content; high palmitic acid, high oleic acid, low linoleic acid, low linolenic acid content; or high palmitic acid, high stearic acid, high oleic acid, low linoleic acid, and low linolenic acid content, relative to a control plant, plant part, plant seed, or plant oil without the saturated molecular marker(s) or the unsaturated molecular marker(s).

The saturated fatty acid content, palmitic acid content, stearic acid content, monounsaturated fatty acid content, oleic acid content, and/or saturated plus monounsaturated fatty acid content, expressed as percent of total fatty acids, in the plants, plant parts, plant seeds, or plant oil having the saturated and unsaturated markers can be greater than that of a control plant, plant part, plant seed, or plant oil without the saturated or unsaturated markers, and the difference (by subtraction) can be about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%.

The polyunsaturated fatty acid content, linoleic acid content, and/or linolenic acid content, expressed as percent of total fatty acids, in the plants, plant parts, plant seeds, or plant oil having the saturated and unsaturated markers can be less than that of a control plant, plant part, plant seed, or plant oil without the saturated or unsaturated markers, and the difference (by subtraction) can be about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%.

The saturated to unsaturated fatty acid composition and or saturated plus monounsaturated to polyunsaturated fatty acid composition, expressed as ratios, in the plants, plant parts, plant seeds, or plant oil having the saturated and unsaturated markers can be greater than those of a control plant, plant part, plant seed, or plant oil without the saturated or unsaturated markers, and the difference (ratios in marker-positive plants, plant parts, plant seeds, or plant oil/ratios in a marker-negative control plant, plant part, plant seed, or plant oil) can be at least about 1%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or 1000%.

In certain embodiments, plants, plant parts, plant seeds, or plant oil having at least one saturated molecular marker and at least one unsaturated molecular marker disclosed herein can comprise a saturated fatty acid content of about 17.5% to about 35% (of total fatty acids) (e.g., about 17.5-20%, 20-22.5%, 22.5-25%, 25-27.5%, 27.5-30%, 30-32.5%, or 32.5-35%) and a polyunsaturated fatty acid content of about 5% to 30% (of total fatty acids) (e.g., about 5-10%, 10-15%, 15-20%, 20-25%, 25-30%) by weight. In certain embodiments, plants, plant parts, plant seeds, or plant oil having at least one saturated molecular marker and at least one unsaturated molecular marker disclosed herein can comprise a palmitic acid content of about 15% to about 30% (e.g., about 15-17.5%, 17.5-20%, 20-22.5%, 22.5-25%, 25-27.5%, 27.5-30%), a stearic acid content of about 2.5% to about 3.5% (e.g., about 2.5-3%, 3-3.5%), an oleic acid content of about 35% to about 80% (e.g., about 35-40%, 40-45%, 45-50%, 50-55%, 55-60%, 65-70%, 75-80%), a linoleic acid content of about 5% to 25% (e.g., about 5-10%, 10-15%, 15-20%, 20-25%), and/or a linolenic acid content of about 1% to about 5% (e.g., about 1-2%, 2-3%, 3-4%, 4-5%) by weight of total fatty acids.

Amount or levels of total fatty acids and specific fatty acids can be measured by any methods for measuring fatty acid amount or levels, including gas chromatography-mass spectrometry (GC-MS) optionally with certain modifications (e.g., with or without initial lipid extraction, with or without isotope labeling of analytes). Fatty acid composition (e.g., percentage of specific fatty acids normalized to total fatty acids) can be calculated based on the amount or concentration of total fatty acids and specific fatty acids in the sample.

In some embodiments, the methods provided herein can produce HPHOLL soybean plants or seeds without a corresponding reduction or penalty in crop yield. The plants described in embodiments herein may have, for example, a yield in excess of 35 bushels per acre.

As disclosed herein, a soybean plant or seed refers to a plant, plant part, or seed of Glycine max (L). In specific embodiments, all chromosomal positions listed herein are identified relative to the reference genome published as the Williams 82 reference genome assembly (Wm82.a2.v1) that can be accessed at the website located at phytozome-next.jgi.doe.gov/info/Gmax_Wm82_a2_v1. See, Schmutz, J., Cannon, S., Schlueter, J. et al. Genome sequence of the palaeopolyploid soybean. Nature 463, 178-183 (2010). The wild perennial soybeans belong to the subgenus Glycine and have a wide array of genetic diversity. The cultivated soybean (Glycine max (L.) Merr.) and its wild annual progenitor (Glycine soja (Sieb. and Zucc.)) belong to the subgenus Soja. The methods described herein can be used in any soybean plant or seed, including but not limited to members of the genus Glycine, for example, Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine sp., Glycine stenophita, Glycine tabacina and Glycine tomentella.

### III. Methods of Introgressing a High Saturated, High Monounsaturated, and/or Low Polyunsaturated Fatty Acid (HPHOLL) QTL

Provided herein are methods for selection and introgression of a saturated and/or unsaturated QTL. The methods for selection and introgression of a saturated QTL can comprise the steps of (a) crossing a first soybean plant comprising a saturated QTL associated with high palmitic acid and/or high stearic acid content with a second soybean plant of a different genotype to produce one or more progeny plants or seeds, and (b) selecting a progeny plant or seed comprising an allele comprising a polymorphic locus associated with said saturated QTL. The methods for selection and introgression of a saturated QTL and an unsaturated QTL can comprise (a) crossing a first soybean plant comprising (i) an saturated QTL associated with high palmitic acid and/or high stearic acid content and (ii) an unsaturated QTL associated with high oleic acid, low linoleic acid, and/or low linolenic acid content with a second soybean plant of a different genotype to produce one or more progeny plants or seeds, and (b) selecting a progeny plant or seed comprising an allele comprising a polymorphic locus associated with said saturated QTL and a polymorphic locus associated with said unsaturated QTL.

The polymorphic locus associated with the saturated QTL can be a chromosomal segment comprising a saturated marker within a genomic region 3567986-9738629 (e.g., a genomic region 4876657-4882865 or 6354365-6359411) of chromosome 8 of a soybean genome. In some embodiments, the saturated QTL is Gm08:063500, Gm08:045000, Gm08:057400, Gm08:072100, Gm08:083900, Gm08:084300, Gm08:092100 or Gm08:126400.

The polymorphic locus linked to said unsaturated QTL can be a chromosomal segment comprising an unsaturated marker within a genomic region 50013483-50015460 of chromosome 10, a genomic region 35315629-35319063 of chromosome 20, a genomic region 45935667-45939896 of chromosome 14, or a genomic region 41419655-41423881 of chromosome 2 of a soybean genome. In some embodiments, the unsaturated QTL associated with high oleic acid, high linoleic acid, and/or low linolenic acid content is Gm10:50014440, Gm20:35318088, Gm14:45937922, Gm14:45937935, and/or Gm02:41422213.

In some embodiments, the polymorphic locus associated with the saturated or unsaturated QTL comprises at least one single nucleotide polymorphisms (SNP), and the saturated or unsaturated marker comprises said at least one SNP.

Selecting the progeny plant or seed from the population is based on the presence of a saturated and/or unsaturated haplotype. In particular embodiments, a saturated and/or unsaturated haplotype comprises alleles of two or more polymorphic loci described herein.

In some embodiments of the method, the SNP associated with the saturated QTL or the saturated marker is a G or an A at position 4879302 of chromosome 8, a T or a C at position 3567986 of chromosome 8, a T or a C at position 4416970 of chromosome 8, an A or a T at position 5521970 of chromosome 8, an A or a T at position 6333332 of chromosome 8, a T or a C at position 6357981 of chromosome 8, a T or a C at position 6958927 of chromosome 8, and/or an A or a G at position 9738629 of chromosome 8 of the soybean genome. The G at position 4879302, the T at position 3567986, the T at position 4416970, the A at position 5521970, the A at position 6333332, the T at position 6357981, the T at position 6958927, or the A at position 9738629 of chromosome 8 of the soybean genome can be associated with high palmitic acid content. In specific embodiments, the SNP associated with the saturated QTL or the saturated marker is a G or an A at position 4879302 of chromosome 8 and/or a T or an C at position 6357981 of chromosome 8 of a genome the soybean plants or seeds, and the G at position 4879302 of chromosome 8 and the T at position 6357981 of chromosome 8 are associated with high palmitic acid content.

In some embodiments, the SNP associated with the unsaturated QTL or the unsaturated marker is an A or a G at position 50014440 of chromosome 10, a G or a C at position 35318088 of chromosome 20, an A or a G at position 45937922 of chromosome 14, an A or a G at position 45937935 of chromosome 14, and/or an A or a G at position 41422213 of chromosome 2 of a genome the soybean plants or seeds. The A at position 50014440 of chromosome 10, the G at position 35318088 of chromosome 20, the A at position 45937922 of chromosome 14, the A at position 45937935 of chromosome 14, and/or the A at position 41422213 of chromosome 2 is associated with high oleic acid, low linoleic acid, and/or low linolenic acid content.

In some embodiments, provided herein are methods for concurrently introgressing at least one or more, two or more, three or more, five or more, six or more, seven or more, eight or more, nine or more, ten or more, eleven or more, or twelve saturated QTLs and/or at least one or more, two or more, three or more, five or more, six or more, seven or more, eight or more, nine or more, ten or more, eleven or more, or twelve unsaturated QTLs, including those identified herein, to generate a population of HPHOLL soybean plants or seeds. In one embodiment, the present disclosure provides a method for introgressing an allele of a polymorphic locus conferring an HPHOLL phenotype.

The methods described herein can be applied to any soybean plant or seed, including but not limited to members of the genus Glycine, for example, Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine sp., Glycine stenophita, Glycine tabacina and Glycine tomentella. In specific embodiments, the saturated and/or unsaturated QTL of the present invention may be introduced into an agronomically elite Glycine max variety. An “agronomically elite” plant, as used herein refers to a plant having a culmination of distinguishable traits such as emergence, vigor, vegetative vigor, disease resistance, seed set, standability, threshability, and yield that allows a producer to harvest a commercially advantageous product.

### IV. Detection/Identification of High Saturated, High Monounsaturated, and/or Low Polyunsaturated Fatty Acid (HPHOLL) Markers and QTLs

Genotyping, e.g., detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

In certain embodiments of the method described herein, genotyping comprises assaying a single nucleotide polymorphism (SNP) marker. SNPs can be assayed and characterized using any of a variety of methods. Such methods include the direct or indirect sequencing of the site, the use of restriction enzymes where the respective alleles of the site create or destroy a restriction site, the use of allele-specific hybridization probes, the use of antibodies that are specific for the proteins encoded by the different alleles of the polymorphism, or by other biochemical interpretation. SNPs can be sequenced using a variation of the chain termination method (Sanger et al., Proc. Natl. Acad. Sci. (U.S.A.) 74: 5463-5467 (1977)) in which the use of radioisotopes are replaced with fluorescently-labeled dideoxy nucleotides and subjected to capillary based automated sequencing (U.S. Pat. No. 5,332,666, the entirety of which is herein incorporated by reference; U.S. Pat. No. 5,821,058, the entirety of which is herein incorporated by reference). Automated sequencers are available from, for example, Applied Biosystems, Foster City, Calif. (3730x1 DNA Analyzer), Beckman Coulter, Fullerton, Calif. (CEQ™ 8000 Genetic Analysis System) and LI-COR, Inc., Lincoln, Nebr. (4300 DNA Analysis System).

The most common marker (e.g., SNP) genotyping methods include hybridization-based (e.g., SNP microarrays), enzyme-based (e.g., primer extension), oligonucleotide ligation, endonuclease cleavage, or a variation of the aforementioned techniques. Primer-extension assays, such as solid-phase minisequencing or pyrosequencing method, a DNA polymerase is used specifically to extend a primer that anneals immediately adjacent to the variant nucleotide. A single labeled nucleoside triphospate complementary to the nucleotide at the variant site is used in the extension reaction. Only those sequences that contain the nucleotide at the variant site will be extended by the polymerase. A primer array can be fixed to a solid support wherein each primer is contained in four small wells, each well being used for one of the four nucleoside triphosphates present in DNA. Template DNA or RNA from each test organism is put into each well and allowed to anneal to the primer. The primer is then extended one nucleotide using a polymerase and a labeled di-deoxy nucleotide triphosphate. The completed reaction can be imaged using devices that are capable of detecting the label which can be radioactive or fluorescent. Using this method several different SNPs can be visualized and detected (Syvänen et al., Hum. Mutat. 13: 1-10 (1999)). The pyrosequencing technique is based on an indirect bioluminometric assay of the pyrophosphate (PPi) that is released from each dNTP upon DNA chain elongation. Following Klenow polymerase mediated base incorporation, PPi is released and used as a substrate, together with adenosine 5-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing procedure (Alderborn et al., Genome Res. 10: 1249-1258 (2000)). An example of an instrument designed to detect and interpret the pyrosequencing reaction is available from Biotage, Charlottesville, Va. (PyroMark MD).

Another marker (e.g., SNP) detection method based on primer-extension assays is a GOOD assay. The GOOD assay (Sauer et al., Nucleic Acids Res. 28: e100 (2000)) is an allele-specific primer extension protocol that employs MALDI-TOF (matrix-assisted laser desorption/ionization time-of-flight) mass spectrometry. The region of DNA containing a SNP is amplified first by PCR amplification. Residual dNTPs are destroyed using an alkaline phosphatase. Allele-specific products are then generated using a specific primer, a conditioned set of a-S-dNTPs and a-S-ddNTPs and a fresh DNA polymerase in a primer extension reaction. Unmodified DNA is removed by 5′ phosphodiesterase digestion and the modified products are alkylated to increase the detection sensitivity in the mass spectrometric analysis. All steps are carried out in a single vial at the lowest practical sample volume and require no purification. The extended reaction can be given a positive or negative charge and is detected using mass spectrometry (Sauer et al., Nucleic Acids Res. 28: e13 (2000)). An instrument in which the GOOD assay is analyzed is for example, the AUTOFLEX® MALDI-TOF system from Bruker Daltonics (Billerica, Mass.).

In one embodiment of the method described herein, genotyping comprises the use of an oligonucleotide probe. The use of an oligonucleotide probe is based on recognition of heteroduplex DNA molecules and includes oligonucleotide hybridization, TAQ-MAN® assays, molecular beacons, electronic dot blot assays and denaturing high-performance liquid chromatography. Oligonucleotide hybridizations can be performed in mass using micro-arrays (Southern, Trends Genet. 12: 110-115 (1996)). TAQ-MAN® assays, or Real Time PCR, detects the accumulation of a specific PCR product by hybridization and cleavage of a double-labeled fluorogenic probe during the amplification reaction. A TAQ-MAN® assay includes four oligonucleotides, two of which serve as PCR primers and generate a PCR product encompassing the polymorphism to be detected. The other two are allele-specific fluorescence-resonance-energy-transfer (FRET) probes. FRET probes incorporate a fluorophore and a quencher molecule in close proximity so that the fluorescence of the fluorophore is quenched. The signal from a FRET probes is generated by degradation of the FRET oligonucleotide, so that the fluorophore is released from proximity to the quencher, and is thus able to emit light when excited at an appropriate wavelength. In the assay, two FRET probes bearing different fluorescent reporter dyes are used, where a unique dye is incorporated into an oligonucleotide that can anneal with high specificity to only one of the two alleles. Useful reporter dyes include 6-carboxy-4,7,2′,7′-tetrachlorofluorecein (TET), 2′-chloro-7′-phenyl-1,4-dichloro-6-carboxyfluorescein (VIC) and 6-carboxyfluorescein phosphoramidite (FAM). A useful quencher is 6-carboxy-N,N,N′,N′-tetramethylrhodamine (TAMRA). Annealed (but not non-annealed) FRET probes are degraded by TAQ DNA polymerase as the enzyme encounters the 5′ end of the annealed probe, thus releasing the fluorophore from proximity to its quencher. Following the PCR reaction, the fluorescence of each of the two fluorescers, as well as that of the passive reference, is determined fluorometrically. The normalized intensity of fluorescence for each of the two dyes will be proportional to the amounts of each allele initially present in the sample, and thus the genotype of the sample can be inferred. An example of an instrument used to detect the fluorescence signal in TAQ-MAN® assays, or Real Time PCR are the 7500 Real-Time PCR System (Applied Biosystems, Foster City, Calif.).

Molecular beacons are oligonucleotide probes that form a stem-and-loop structure and possess an internally quenched fluorophore. When they bind to complementary targets, they undergo a conformational transition that turns on their fluorescence. These probes recognize their targets with higher specificity than linear probes and can easily discriminate targets that differ from one another by a single nucleotide. The loop portion of the molecule serves as a probe sequence that is complementary to a target nucleic acid. The stem is formed by the annealing of the two complementary arm sequences that are on either side of the probe sequence. A fluorescent moiety is attached to the end of one arm and a nonfluorescent quenching moiety is attached to the end of the other arm. The stem hybrid keeps the fluorophore and the quencher so close to each other that the fluorescence does not occur. When the molecular beacon encounters a target sequence, it forms a probe-target hybrid that is stronger and more stable than the stem hybrid. The probe undergoes spontaneous conformational reorganization that forces the arm sequences apart, separating the fluorophore from the quencher, and permitting the fluorophore to fluoresce (Bonnet et al., 1999). The power of molecular beacons lies in their ability to hybridize only to target sequences that are perfectly complementary to the probe sequence, hence permitting detection of single base differences (Kota et al., Plant Mol. Biol. Rep. 17: 363-370 (1999)). Molecular beacon detection can be performed for example, on the Mx4000@Multiplex Quantitative PCR System from Stratagene (La Jolla, Calif.).

In one embodiment, the SNP marker described in the methods provided herein can be identified by a corresponding nucleic acid molecule (e.g., oligonucleotide probe) that comprises at least 15 nucleotides and has at least at least 90% (90%, 91%, 92%, 93%, 94%, 95%, 96%, 97%, 98%, 99%, or 100%) sequence identity to a sequence of the same number of consecutive nucleotides in either sense or antisense strand of DNA that include or are immediately adjacent to the SNP in the soybean genome. For example, the deletion marker disclosed herein is capable of being identified by a corresponding nucleic acid molecule that comprises at least 15 nucleotides that include or are immediately adjacent to the deletion, or by a nucleic acid molecule that only binds to the unique junction formed by the deletion event. In some embodiments, the SNP markers can be detected using a pair of primers, i.e., a first primer and a second primer each comprising at least 15 nucleotides. In some embodiments, the first primer has at least 90% sequence identity to a sequence of the same number of contiguous nucleotides of a sense DNA strand of a region comprising or adjacent to the SNP marker, and the second primer has at least 90% sequence identity to a sequence of the same number of contiguous nucleotides of an antisense DNA strand of the region comprising or adjacent to the SNP marker. In some embodiments, a saturated SNP marker is located in a genomic region 3567986-9738629 (e.g., genomic region 4876657-4882865 or 6354365-6359411) of chromosome 8 of the soybean genome. In some embodiments, an unsaturated SNP marker is located in a genomic region 50013483-50015460 of chromosome 10, a genomic region 35315629-35319063 of chromosome 20, a genomic region 45935667-45939896 of chromosome 14, or a genomic region 41419655-41423881 of chromosome 2 of the soybean genome. The saturated SNP markers can be a G at position 4879302, a T at position 3567986, a T at position 4416970, an A at position 5521970, an A at position 6333332, a T at position 6357981, a T at position 6958927, or an A at position 9738629 of chromosome 8 of the soybean genome. The unsaturated SNP marker can be an A at position 50014440 of chromosome 10, a G at position 35318088 of chromosome 20, an A at position 45937922 of chromosome 14, an A at position 45937935 of chromosome 14, and/or an A at position 41422213 of chromosome 2 of the soybean genome. Accordingly, in some embodiments, the saturated SNP markers provided herein can be detected using an oligonucleotide probe comprising a nucleic acid sequence having at least 90% sequence identity to any one of NOs: 1, 2, 5, 6, 9, 10, 13, 14, 17, 18, 21, 22, 25, 26, 29, and 30 or nucleic acid sequence of any one of NOs: 1, 2, 5, 6, 9, 10, 13, 14, 17, 18, 21, 22, 25, 26, 29, and 30; or first and second primers comprising nucleic acid sequences having at least 90% sequence identity to a pair of: SEQ ID NOs: 3 and 4; SEQ ID NOs: 7 and 8, SEQ ID NOs: 11 and 12, SEQ ID NOs: 15 and 16, SEQ ID NOs: 19 and 20, SEQ ID NOs: 23 and 24, SEQ ID NOs: 27 and 28, or SEQ ID NOs: 31 and 32; or a nucleic acid sequence of SEQ ID NOs: 3 and 4; SEQ ID NOs: 7 and 8, SEQ ID NOs: 11 and 12, SEQ ID NOs: 15 and 16, SEQ ID NOs: 19 and 20, SEQ ID NOs: 23 and 24, SEQ ID NOs: 27 and 28, or SEQ ID NOs: 31 and 32. The unsaturated SNP markers provided herein using an oligonucleotide probe comprising a nucleic acid sequence having at least 90% sequence identity to any one of NOs: 33, 34, 37, 38, 41, 42, 45, 46, 49, and 50 or nucleic acid sequence of any one of NOs: 33, 34, 37, 38, 41, 42, 45, 46, 49, and 50; or first and second primers comprising a nucleic acid sequence having at least 90% sequence identity to a pair of: SEQ ID NOs: 35 and 36, 39 and 40, or 43 and 44, 47 and 48, or 51 and 52; or a nucleic acid sequence of SEQ ID NOs: 35 and 36, 39 and 40, or 43 and 44, 47 and 48, or 51 and 52.

The electronic dot blot assay uses a semiconductor microchip comprised of an array of microelectrodes covered by an agarose permeation layer containing streptavidin. Biotinylated amplicons are applied to the chip and electrophoresed to selected pads by positive bias direct current, where they remain embedded through interaction with streptavidin in the permeation layer. The DNA at each pad is then hybridized to mixtures of fluorescently labeled allele-specific oligonucleotides. Single base pair mismatched probes can then be preferentially denatured by reversing the charge polarity at individual pads with increasing amperage. The array is imaged using a digital camera and the fluorescence quantified as the amperage is ramped to completion. The fluorescence intensity is then determined by averaging the pixel count values over a region of interest (Gilles et al., Nature Biotech. 17: 365-370 (1999)).

A more recent application based on recognition of heteroduplex DNA molecules uses denaturing high-performance liquid chromatography (DHPLC). This technique represents a highly sensitive and fully automated assay that incorporates a Peltier-cooled 96-well autosampler for high-throughput SNP analysis. It is based on an ion-pair reversed-phase high performance liquid chromoatography method. The heart of the assay is a polystyrene-divinylbenzene copolymer, which functions as a stationary phase. The mobile phase is composed of an ion-pairing agent, triethylammonium acetate (TEAA) buffer, which mediates the binding of DNA to the stationary phase, and an organic agent, acetonitrile (ACN), to achieve subsequent separation of the DNA from the column. A linear gradient of CAN allows the separation of fragments based on the presence of heteroduplexes. DHPLC thus identifies mutations and polymorphisms that cause heteroduplex formation between mismatched nucleotides in double-stranded PCR-amplified DNA. In a typical assay, sequence variation creates a mixed population of heteroduplexes and homoduplexes during reannealing of wild-type and mutant DNA. When this mixed population is analyzed by DHPLC under partially denaturing temperatures, the heteroduplex molecules elute from the column prior to the homoduplex molecules, because of their reduced melting temperatures (Kota et al., Genome 44: 523-528 (2001)). An example of an instrument used to analyze SNPs by DHPLC is the WAVE® HS System from Transgenomic, Inc. (Omaha, Nebr.).

A microarray-based method for high-throughput monitoring of plant gene expression can be utilized as a genetic marker system. This ‘chip’-based approach involves using microarrays of nucleic acid molecules as gene-specific hybridization targets to quantitatively or qualitatively measure expression of plant genes (Schena et al., Science 270:467-470 (1995), the entirety of which is herein incorporated by reference; Shalon, Ph.D. Thesis. Stanford University (1996), the entirety of which is herein incorporated by reference). Every nucleotide in a large sequence can be queried at the same time. Hybridization can be used to efficiently analyze nucleotide sequences. Such microarrays can be probed with any combination of nucleic acid molecules. Particularly preferred combinations of nucleic acid molecules to be used as probes include a population of mRNA molecules from a known tissue type or a known developmental stage or a plant subject to a known stress (environmental or man-made) or any combination thereof (e.g. mRNA made from water stressed leaves at the 2 leaf stage). Expression profiles generated by this method can be utilized as markers.

Polymorphisms can also be identified by Single Strand Conformation Polymorphism (SSCP) analysis. SSCP is a method capable of identifying most sequence variations in a single strand of DNA, typically between 150 and 250 nucleotides in length (Elles, Methods in Molecular Medicine: Molecular Diagnosis of Genetic Diseases, Humana Press (1996); Orita et al., Genomics 5: 874-879 (1989)). Under denaturing conditions, a single strand of DNA will adopt a conformation that is uniquely dependent on its sequence conformation. This conformation usually will be different, even if only a single base is changed. Most conformations have been reported to alter the physical configuration or size sufficiently to be detectable by electrophoresis.

In one embodiment of the method described herein, the oligonucleotide probe is adjacent to a polymorphic nucleotide position in the saturated or unsaturated QTL. For the purpose of QTL mapping, the markers included must be diagnostic of origin in order for inferences to be made about subsequent populations. SNP markers are ideal for mapping because the likelihood that a particular SNP allele is derived from independent origins in the extant populations of a particular species is very low. As such, SNP markers are useful for tracking and assisting introgression of QTLs, particularly in the case of haplotypes. In one embodiment of the method described herein, genotyping comprises detecting a haplotype.

GEMMA GWAS methods can be used to identify the top genomic regions (QTL) associated with the HPHOLL trait.

A maximum likelihood estimate (ILE) for the presence of a marker is calculated, together with an MLE assuming no QTL effect, to avoid false positives. A log 10 of an odds ratio (LOD) is then calculated as: LOD=log 10 (MLE for the presence of a QTL/MLE given no linked QTL). The LOD score essentially indicates how much more likely the data are to have arisen assuming the presence of a QTL versus in its absence. The LOD threshold value for avoiding a false positive with a given confidence, say 95%, depends on the number of markers and the length of the genome. Graphs indicating LOD thresholds are set forth in Lander and Botstein, Genetics, 121:185-199 (1989), and further described by Arns and Moreno-Gonzalez, Plant Breeding, Hayward, Bosemark, Romagosa (eds.) Chapman & Hall, London, pp. 314-331 (1993).

Additional models can be used for marker and QTL detection. Many modifications and alternative approaches to interval mapping have been reported, including the use of non-parametric methods (Kruglyak and Lander, Genetics, 139:1421-1428 (1995), the entirety of which is herein incorporated by reference). Multiple regression methods or models can also be used, in which the trait is regressed on a large number of markers (Jansen, Biometrics in Plant Breed, van Oijen, Jansen (eds.) Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval, and at the same time onto a number of markers that serve as ‘cofactors,’ have been reported by Jansen and Stam, Genetics, 136:1447-1455 (1994) and Zeng, Genetics, 136:1457-1468 (1994). Generally, the use of cofactors reduces the bias and sampling error of the estimated QTL positions (Utz and Melchinger, Biometrics in Plant Breeding, van Oijen, Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng, Genetics, 136:1457-1468 (1994)). These models can be extended to multi-environment experiments to analyze genotype-environment interactions (Jansen et al., Theo. Appl. Genet. 91:33-37 (1995).

Selection of appropriate mapping populations is important to map construction. The choice of an appropriate mapping population depends on the type of marker systems employed (Tanksley et al., Molecular mapping of plant chromosomes. chromosome structure and function: Impact of new concepts J. P. Gustafson and R. Appels (eds.). Plenum Press, New York, pp. 157-173 (1988), the entirety of which is herein incorporated by reference). Consideration must be given to the source of parents (adapted vs. exotic) used in the mapping population. Chromosome pairing and recombination rates can be severely disturbed (suppressed) in wide crosses (adapted×exotic) and generally yield greatly reduced linkage distances. Wide crosses will usually provide segregating populations with a relatively large array of polymorphisms when compared to progeny in a narrow cross (adapted×adapted).

An F2 population is the first generation of selfing after the hybrid seed is produced. Usually a single F1 plant is selfed to generate a population segregating for all the genes in Mendelian (1:2:1) fashion. Maximum genetic information is obtained from a completely classified F2 population using a codominant marker system (Mather, Measurement of Linkage in Heredity: Methuen and Co., (1938), the entirety of which is herein incorporated by reference). In the case of dominant markers, progeny tests (e.g., F3, BCF2) are required to identify the heterozygotes, thus making it equivalent to a completely classified F2 population. However, this procedure is often prohibitive because of the cost and time involved in progeny testing. Progeny testing of F2 individuals is often used in map construction where phenotypes do not consistently reflect genotype (e.g. disease resistance) or where trait expression is controlled by a QTL. Segregation data from progeny test populations (e.g. F3 or BCF2) can be used in map construction. Marker-assisted selection can then be applied to cross progeny based on marker-trait map associations (F2, F3), where linkage groups have not been completely disassociated by recombination events (i.e., maximum disequilibrium).

In certain embodiments, additional markers linked to a saturated or unsaturated allele. This may be carried out, for example, by first preparing an F2 population by selfing an F1 hybrid produced by crossing inbred varieties only one of which comprises a saturated and/or unsaturated allele conferring HPHOLL content. Recombinant inbred lines (RIL) (genetically related lines, usually F5 or progeny thereof, developed from continuously selfing F2 lines towards homozygosity) can then be prepared and used as a mapping population. Information obtained from dominant markers can be maximized by using RIL because all or nearly loci are homozygous. The genetic linkage of additional marker molecules can be established by a gene mapping model such as, without limitation, the flanking marker model reported by Lander and Botstein, Genetics, 121:185-199 (1989), and the interval mapping, based on maximum likelihood methods described by Lander and Botstein, Genetics, 121:185-199 (1989), and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y., the manual of which is herein incorporated by reference in its entirety). Use of Qgene software is a particularly preferred approach.

Backcross populations (e.g., generated from a cross between a desirable variety (recurrent parent) and another variety (donor parent) carrying a trait not present in the former can also be utilized as a mapping population. A series of backcrosses to the recurrent parent can be made to recover most of its desirable traits. Thus a population is created consisting of individuals similar to the recurrent parent but each individual carries varying amounts of genomic regions from the donor parent. Backcross populations can be useful for mapping dominant markers if all loci in the recurrent parent are homozygous and the donor and recurrent parent have contrasting polymorphic marker alleles (Reiter et al., 1992).

Useful populations for mapping purposes are near-isogenic lines (NIL). NILs are created by many backcrosses to produce an array of individuals that are nearly identical in genetic composition except for the desired trait or genomic region can be used as a mapping population. In mapping with NILs, only a portion of the polymorphic loci are expected to map to a selected region. Mapping may also be carried out on transformed plant lines.

In one embodiment, the method further comprises determining the fatty acid content (e.g., specific fatty acid compositions normalized to the total fatty acids) of the second population of soybean plants or seeds, wherein the second population of soybean plants or seeds is progeny soybean plants or seeds produced from the first population of soybean plants or seeds comprising one or more alleles comprising one or more saturated and/or unsaturated QTLs. The saturated QTL can be one or more of Gm08:063500, Gm08:045000, Gm08:057400, Gm08:072100, Gm08:083900, Gm08:084300, Gm08:092100 and/or Gm08:126400. The unsaturated QTL can be one or more of Gm10:50014440, Gm20:35318088, Gm14:45937922, Gm14:45937935, and/or Gm02:41422213. Amount or levels of total fatty acids and specific fatty acids can be measured by any methods for measuring fatty acid amount or levels, including gas chromatography-mass spectrometry (GC-MS) optionally with certain modifications (e.g., with or without initial lipid extraction, with or without isotope labeling of analytes). Fatty acid composition (e.g., percentage of specific fatty acids normalized to total fatty acids) can be calculated based on the amount or concentration of total fatty acids and specific fatty acids in the sample.

### Nucleic Acid Molecules for Detecting a Molecular Marker in Soybean Genome

Provided herein is a nucleic acid molecule for detecting a saturated or unsaturated molecular marker (e.g., associated with a HPHOLL phenotype) in soybean DNA. In some embodiments, the nucleic acid molecule is an oligonucleotide probe. In some embodiments, the nucleic acid molecule comprises at least 15 nucleotides and has at least 90% (91%, 92%, 93%, 94%, 95%, 96%, 97%, 98%, 99%, or 100%) sequence identity to a sequence of the same number of consecutive nucleotides in a sense or antisense strand of DNA in a region comprising or adjacent (e.g., immediately adjacent) to the molecular marker. In some embodiments, the saturated molecular marker is located in a genomic region 3567986-9738629 (e.g., a genomic region 4876657-4882865 or 6354365-6359411) of chromosome 8, and the unsaturated molecular marker is located in a genomic region 50013483-50015460 of chromosome 10, a genomic region 35315629-35319063 of chromosome 20, a genomic region 45935667-45939896 of chromosome 14, or a genomic region 41419655-41423881 of chromosome 2 of the soybean genome. In some embodiments, the molecular marker is an SNP marker. Exemplary saturated SNP markers include: a G or an A at position 4879302, a T or a C at position 3567986, a T or a C at position 4416970, an A or a T at position 5521970, an A or a T at position 6333332, a T or a C at position 6357981, a T or a C at position 6958927, and an A or a G at position 9738629 of chromosome 8 of the soybean genome. Exemplary unsaturated SNP markers include: an A or a G at position 50014440 of chromosome 10; a G or a C at position 35318088 of chromosome 20; an A or a G at position 45937922 of chromosome 14; an A or a G at position 45937935 of chromosome 14, and an A or a G at position 41422213 of chromosome 2 of the soybean genome. In some embodiments, the nucleic acid molecule (e.g., an oligonucleotide probe) described herein comprises any one of SEQ ID NOs: 1, 2, 5, 6, 9, 10, 13, 14, 17, 18, 21, 22, 25, 26, 29, and 30 for detection of a saturated marker, and any one of SEQ ID NOs: 33, 34, 37, 38, 41, 42, 45, 46, 49, and 50 for detection of an unsaturated marker. The nucleic acid molecule can comprise a nucleic acid sequence having at least 90% sequence identity to any one of SEQ ID NOs: 1, 2, 5, 6, 9, 10, 13, 14, 17, 18, 21, 22, 25, 26, 29, 30, 33, 34, 37, 38, 41, 42, 45, 46, 49, and 50. The nucleic acid molecule can further comprise a detectable label, e.g., a fluorescent label or a radioactive label.

Also provided herein is a pair of nucleic acid molecules (e.g., a pair of primers) for detecting a saturated or unsaturated molecular marker (e.g., associated with a HPHOLL phenotype) by primer extension method, e.g., PCR. The pair of nucleic acid molecules can comprise a first primer and a second primer each comprising at least 15 nucleotides, with the first primer having at least 90% sequence identity to a sequence of the same number of contiguous nucleotides of a sense DNA strand of a region comprising or adjacent to the molecular marker, and the second primer having at least 90% sequence identity to a sequence of the same number of contiguous nucleotides of an antisense DNA strand of the region comprising or adjacent to the molecular marker. In some embodiments, the saturated molecular marker is located in a genomic region 3567986-9738629 (e.g., 4876657-4882865, 6354365-6359411) of chromosome 8, and the unsaturated molecular marker is located in a genomic region 50013483-50015460 of chromosome 10, a genomic region 35315629-35319063 of chromosome 20, a genomic region 45935667-45939896 of chromosome 14, or a genomic region 41419655-41423881 of chromosome 2 of a soybean genome. The pair of primers can be used to detect the presence or absence of a saturated SNP marker, e.g., a G or an T at position 4879302, a T or a C at position 3567986, a T or a C at position 4416970, an A or a T at position 5521970, an A or a T at position 6333332, a T or a C at position 6357981, a T or a C at position 6958927, or an A or a G at position 9738629 of chromosome 8; or the presence or absence of an unsaturated SNP marker, e.g., an A or a G at position 50014440 of chromosome 10, a G or a C at position 35318088 of chromosome 20, an A or a G at position 45937922 of chromosome 14, an A or a G at position 45937935 of chromosome 14, and/or an A or a G at position 41422213 of chromosome 2 of the soybean genome. In some embodiments, the first and second primers comprise nucleic acid sequences having at least 90% identity to any one pair of: SEQ ID NOs: 3 and 4; SEQ ID NOs: 7 and 8, SEQ ID NOs: 11 and 12, SEQ ID NOs: 15 and 16, SEQ ID NOs: 19 and 20, SEQ ID NOs: 23 and 24, SEQ ID NOs: 27 and 28, or SEQ ID NOs: 31 and 32; or a nucleic acid sequence of SEQ ID NOs: 3 and 4; SEQ ID NOs: 7 and 8, SEQ ID NOs: 11 and 12, SEQ ID NOs: 15 and 16, SEQ ID NOs: 19 and 20, SEQ ID NOs: 23 and 24, SEQ ID NOs: 27 and 28, or SEQ ID NOs: 31 and 32 to detect a saturated SNP marker; or comprise nucleic acid sequences having at least 90% identity to any one pair of: SEQ ID NOs: 35 and 36, 39 and 40, or 43 and 44, 47 and 48, or 51 and 52; or a nucleic acid sequence of SEQ ID NOs: 35 and 36, 39 and 40, or 43 and 44, 47 and 48, or 51 and 52 to detect an unsaturated SNP marker.

### V. Breeding of Soybean Plants Comprising High Saturated, High Monounsaturated, and/or Low Polyunsaturated Fatty Acid (HPHOLL) Content

HPHOLL soybean plants of the present disclosure can be part of or generated from a breeding program. The choice of breeding method depends on the mode of plant reproduction, the heritability of the trait(s) being improved, and the type of cultivar used commercially (e.g., F1 hybrid cultivar, pureline cultivar, etc.). A cultivar is a race or variety of a plant that has been created or selected intentionally and maintained through cultivation.

Descriptions of breeding methods that are commonly used for different crops can be found in one of several reference books, see, e.g., Allard, Principles of Plant Breeding, John Wiley & Sons, NY, U. of CA, Davis, Calif., 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

Selected, non-limiting approaches for breeding the plants of the present invention are set forth below. A breeding program can be enhanced using marker assisted selection (MAS) of the progeny of any cross. It is further understood that any commercial and non-commercial cultivars can be utilized in a breeding program. Factors such as, for example, emergence vigor, vegetative vigor, stress tolerance, disease resistance, branching, flowering, seed set, seed size, seed density, standability, and threshability etc. will generally dictate the choice.

For highly heritable traits, a choice of superior individual plants evaluated at a single location will be effective, whereas for traits with low heritability, selection should be based on mean values obtained from replicated evaluations of families of related plants. Popular selection methods commonly include pedigree selection, modified pedigree selection, mass selection, and recurrent selection. In a preferred embodiment a backcross or recurrent breeding program is undertaken.

The complexity of inheritance influences choice of the breeding method. Backcross breeding can be used to transfer one or a few favorable genes for a highly heritable trait into a desirable cultivar. This approach has been used extensively for breeding disease-resistant cultivars. Various recurrent selection techniques are used to improve quantitatively inherited traits controlled by numerous genes. The use of recurrent selection in self-pollinating crops depends on the ease of pollination, the frequency of successful hybrids from each pollination event, and the number of hybrid offspring from each successful cross.

Breeding lines can be tested and compared to appropriate standards in environments representative of the commercial target area(s) for two or more generations. The best lines are candidates for new commercial cultivars; those still deficient in traits may be used as parents to produce new populations for further selection.

One method of identifying a superior plant is to observe its performance relative to other experimental plants and to a widely grown standard cultivar. If a single observation is inconclusive, replicated observations can provide a better estimate of its genetic worth. A breeder can select and cross two or more parental lines, followed by repeated selfing and selection, producing many new genetic combinations.

The development of new soybean cultivars requires the development and selection of soybean varieties, the crossing of these varieties and selection of superior hybrid crosses. The hybrid seed can be produced by manual crosses between selected male-fertile parents or by using male sterility systems. Hybrids are selected for certain single gene traits such as pod color, flower color, seed yield, pubescence color or herbicide resistance which indicate that the seed is truly a hybrid. Additional data on parental lines, as well as the phenotype of the hybrid, influence the breeder's decision whether to continue with the specific hybrid cross.

Pedigree breeding and recurrent selection breeding methods can be used to develop cultivars from breeding populations. Breeding programs combine desirable traits from two or more cultivars or various broad-based sources into breeding pools from which cultivars are developed by selfing and selection of desired phenotypes. New cultivars can be evaluated to determine which have commercial potential.

Pedigree breeding is used commonly for the improvement of self-pollinating crops. Two parents who possess favorable, complementary traits (e.g., HPHOLL) are crossed to produce an F1. An F2 population is produced by selfing one or several F1's. Selection of the best individuals in the best families is selected. Replicated testing of families can begin in the F4 generation to improve the effectiveness of selection for traits with low heritability. At an advanced stage of inbreeding (i.e., F6 and F7), the best lines or mixtures of phenotypically similar lines are tested for potential release as new cultivars.

Backcross breeding has been used to transfer genes for a simply inherited, highly heritable trait into a desirable homozygous cultivar or inbred line, which is the recurrent parent. The source of the trait to be transferred is called the donor parent. The resulting plant is expected to have the attributes of the recurrent parent (e.g., cultivar) and the desirable trait transferred from the donor parent. After the initial cross, individuals possessing the phenotype of the donor parent are selected and repeatedly crossed (backcrossed) to the recurrent parent. The resulting parent is expected to have the attributes of the recurrent parent (e.g., cultivar) and the desirable trait transferred from the donor parent.

The single-seed descent procedure in the strict sense refers to planting a segregating population, harvesting a sample of one seed per plant, and using the one-seed sample to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the F2 plants originally sampled in the population will be represented by a progeny when generation advance is completed.

In a multiple-seed procedure, soybean breeders commonly harvest one or more pods from each plant in a population and thresh them together to form a bulk. Part of the bulk is used to plant the next generation and part is put in reserve. The procedure has been referred to as modified single-seed descent or the pod-bulk technique.

The multiple-seed procedure has been used to save labor at harvest. It is considerably faster to thresh pods with a machine than to remove one seed from each by hand for the single-seed procedure. The multiple-seed procedure also makes it possible to plant the same number of seed of a population each generation of inbreeding.

Descriptions of other breeding methods that are commonly used for different traits and crops can be found in one of several reference books (e.g., Fehr, Principles of Cultivar Development Vol. 1, pp. 2-3 (1987)).

### VI. Soybean Plants, Soybean Seeds, Soybean Oil, and Soybean Products

Provided herein is a soybean plant or soybean seed selected, generated, or produced by any methods disclosed herein and having HPHOLL characteristics (e.g., comprising high palmitic acid, high oleic acid, high linoleic acid, and/or low linolenic acid content relative to a control plant or seed). In some embodiments, such HPHOLL soybean plant or seed comprises one or more saturated QTLs and/or one or more unsaturated QTLs. A saturated QTL of the soybean plant or soybean seed can be Gm08:063500, Gm08:045000, Gm08:057400, Gm08:072100, Gm08:083900, Gm08:084300, Gm08:092100 and/or Gm08:126400. A saturated SNP marker can be a G or an A at position 4879302, a T or a C at position 3567986, a T or a C at position 4416970, an A or a T at position 5521970, an A or a T at position 6333332, a T or a C at position 6357981, a T or a C at position 6958927, an A or a G at position 9738629 of chromosome 8 of the soybean genome, with the G at position 4879302, the T at position 3567986, the T at position 4416970, the A at position 5521970, the A at position 6333332, the T at position 6357981, the T at position 6958927, or the A at position 9738629 of chromosome 8 being associated with high palmitic acid content. An unsaturated QTL of the soybean plant or soybean seed can be Gm10:50014440, Gm20:35318088, Gm14:45937922, Gm14:45937935, and/or Gm02:41422213. An unsaturated SNP marker can be an A or a G at position 50014440 of chromosome 10; a G or a C at position 35318088 of chromosome 20; an A or a G at position 45937922 of chromosome 14; an A or a G at position 45937935 of chromosome 14, and/or an A or a G at position 41422213 of chromosome 2 of the soybean genome, with the A at position 50014440 of chromosome 10, the G at position 35318088 of chromosome 20, the A at position 45937922 of chromosome 14, the A at position 45937935 of chromosome 14, and/or the A at position 41422213 of chromosome 2 being associated with high oleic acid, low linoleic acid, and/or low linolenic acid content.

Also provided herein is a population of soybean plants or soybean seeds selected, generated, or produced by any methods disclosed herein and having HPHOLL characteristics. In some embodiments, such population of HPHOLL soybean plants or seeds comprises one or more saturated QTLs and/or one or more unsaturated QTLs at a greater frequency relative to a control population of soybean plants or seeds not having HPHOLL characteristics.

Also provided herein is a population of soybean plants or soybean seeds comprising at least one saturated QTL and/or at least one unsaturated QTL disclosed herein at a greater frequency than a control population of soybean plants or seeds. Such population of soybean plants or seeds comprising a saturated or unsaturated QTL at a greater frequency can comprise HPHOLL characteristics.

In some embodiments, a control population of soybean plants or seeds is a population produced by methods without assaying for or selecting based on a saturated or unsaturated molecular marker disclosed herein. The HPHOLL soybean plants and seeds of the present disclosure includes soybean plants and seeds that contain a saturated or unsaturated molecular marker disclosed herein, as well as soybean plants and seeds that do not contain a saturated or unsaturated molecular marker disclosed herein. The HPHOLL soybean plants and seeds of the present disclosure can be produced, exclusively or nonexclusively, from plants or seeds that contain a saturated or unsaturated molecular marker disclosed herein, or can be produced, exclusively or nonexclusively, from plants or seeds that do not contain a saturated or unsaturated molecular marker disclosed herein.

Also provided herein is oil produced from soybean plants or seeds of the present disclosure. Such oil can comprise HPHOLL characteristics and/or one or more saturated and/or unsaturated molecular markers, as described in detail elsewhere in the present disclosure.

Also provided herein are soybean plant parts (e.g., seed, juice, pulp, fruit, flowers, nectar, embryos, pollen, ovules, leaves, stems, branches, kernels, stalks, roots, root tips, anthers, etc.) and plant products produced from soybean plants or seeds of the present disclosure. “Plant products”, as used herein, refers to any product or composition produced from the plant, including plait oil, plant extract (e.g., fatty acids, sweetener, antioxidants, alkaloids, etc.), plant protein, plant concentrate (e.g., whole plant concentrate or plant part concentrate), plant powder (e.g., formulated powder, such as formulated plant part powder (e.g., seed flour)), plant biomass (e.g., dried biomass, such as crushed and/or powdered biomass), food and beverage products, soap, cosmetics, ink, paint, and industrial materials. The plant parts and plant products provided herein can comprise HPHOLL characteristics and/or one or more saturated and/or unsaturated molecular markers of the present disclosure.

Soybeans and oils provided herein can be suitable for use in a variety of soyfoods made from whole soybeans, such as edamame, soymilk, soy nut butter, natto, and tempeh, and soyfoods made from processed soybeans and soybean oil, including soybean meal, soy flour, soy protein concentrate, soy protein isolates, texturized soy protein concentrate, hydrolyzed soy protein, whipped topping, cooking oil, salad oil, shortening, and lecithin. Soymilk, which is typically produced by soaking and grinding whole soybeans, may be consumed as is, spray-dried, or processed to form soy yogurt, soy cheese, tofu, or yuba. The present soybean or oil may be advantageously used in these and other soyfoods because of its improved oxidative stability, the reduction of off-flavor precursors, and its high saturated fatty acid level.

Soybean Plants, Soybean Seeds, Soybean Parts, and Soybean Products Comprising a Saturated and/or Unsaturated Marker

In some embodiments, soybean plants, soybean parts, soybean seeds, and soybean products (e.g., soybean oil) of the present disclosure contain at least one saturated molecular marker and/or at least one unsaturated molecular marker. Soybean plants, soybean parts, soybean seeds, and soybean products (e.g., soybean oil) comprising at least one saturated molecular marker (e.g., SNP or deletion marker) can comprise higher saturated fatty acid content as compared to a control plant, plant part, seed, or product (e.g., oil) without the saturated molecular marker. In some embodiments, the soybean plants, soybean parts, soybean seeds, or soybean products (e.g., soybean oil) having at least one saturated molecular marker disclosed herein can have higher saturated fatty acid content, expressed as percent of total fatty acids, as compared to a control plant, plant part, seed, or product (e.g., oil) without the saturated molecular marker, expressed as percent of total fatty acids, and the difference (by subtraction) can be at least about 1%, 5%, 10%, 15%, 20%, 25%, 30%, 35%, 40%, 45%, 50%, 55%, 60%, 65%, 70%, 75%, 80%, 85%, 90%, 95%, or 99%. In some embodiments, the saturated fatty acid content in soybean plants, soybean parts, soybean seeds, and soybean products (e.g., soybean oil) comprising at least one saturated molecular marker is at least about 1%, 2%, 3%, or 4% greater than that in a control plant, plant part, seed, or product (e.g., oil) without the saturated molecular marker, expressed as difference in % of total fatty acid in dry weight. In certain embodiments, soybean plants, soybean parts, soybean seeds, or soybean products (e.g., soybean oil) having at least one saturated molecular marker (e.g., SNP or deletion marker) disclosed herein comprise at least about 4 percent increase in palmitic acid content or about 0.5 percent increase in stearic acid content compared to a control plant, plant part, seed, or product (e.g., oil) without the saturated molecular marker, expressed as difference in % of total fatty acid in dry weight. In some embodiments, the soybean plants, soybean parts, soybean seeds, or soybean products (e.g., soybean oil) having at least one saturated molecular marker disclosed herein can comprise a saturated fatty acid content of about 17.5% to about 35%, e.g., about 17.5-20%, 20-22.5%, 22.5-25%, 25-27.5%, 27.5-30%, 30-32.5%, or 32.5-35% (of total fatty acids) by weight. In some embodiments, the percentage of saturated fatty acids in an oil composition of the present invention is 15% or less; 14% or less; 13% or less; 12% or less, 11% or less; 10% or less; 9% or less; 8% or less; 7% or less; 6% or less; 5% or less; 4% or less; or 3.6% or less; or is a range from 2 to 3%; 2 to 3.6%; 2 to 4%; 2 to 8%; 3 to 15%; 3 to 10%; 3 to 8%; 3 to 6%; 3.6 to 7%; 5 to 8%; 7 to 10%; or 10 to 15%. In some embodiments, the soybean plants, soybean parts, soybean seeds, or soybean products (e.g., soybean oil) having at least one saturated molecular marker disclosed herein can comprise a palmitic acid content of about 15% to about 30% (e.g., about 15-17.5%, 17.5-20%, 20-22.5%, 22.5-25%, 25-27.5%, 27.5-30%) and/or a stearic acid content of about 2.5% to about 3.5% (e.g., about 2.5-3%, 3-3.5%).

In some embodiments, soybean plants, soybean parts, soybean seeds, and soybean products (e.g., soybean oil) comprising at least one unsaturated molecular marker (e.g., SNP or deletion marker) can comprise higher saturated fatty acid content as compared to a control plant, plant part, seed, or product (e.g., oil) without the unsaturated molecular marker. In some embodiments, the soybean plants, soybean parts, soybean seeds, or soybean products (e.g., soybean oil) having at least one unsaturated molecular marker (e.g., SNP or deletion marker) disclosed herein can have higher monounsaturated fatty acid content, expressed as percent of total fatty acids, as compared to that of a control plant, plant part, seed, or product (e.g., oil) without the unsaturated molecular marker, expressed as percent of total fatty acids, and the difference (by subtraction) can be at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%. In some embodiments, the soybean plants, soybean parts, soybean seeds, or soybean product (e.g., soybean oil) having at least one unsaturated molecular marker (e.g., SNP or deletion marker) disclosed herein can have lower polyunsaturated fatty acid content, expressed as percent of total fatty acids, as compared to that of a control plant, plant part, seed, or product (e.g., oil) without the unsaturated molecular marker, expressed as percent of total fatty acids, and the difference (by subtraction) can be at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%.

In certain embodiments, soybean plants, soybean parts, soybean seeds, or soybean products (e.g., soybean oil) having at least one saturated molecular marker (e.g., SNP or deletion marker) and at least one unsaturated molecular marker (e.g., SNP or deletion marker) disclosed herein can comprise HPHOLL characteristics, including a greater saturated fatty acid content (e.g., a greater palmitic acid and/or stearic acid content), a greater monounsaturated fatty acid content (e.g., a greater oleic acid content), a lower polyunsaturated fatty acid content (e.g., a less linoleic acid and/or linolenic acid content), a greater saturated to unsaturated fatty acid composition, a greater saturated plus monounsaturated to polyunsaturated fatty acid composition, and combination or variation of any thereof, as compared to a control plant, plant part, seed, or product (e.g., oil) without the saturated molecular marker(s) or the unsaturated molecular marker(s). For example, soybean plants, soybean parts, soybean seeds, or soybean product (e.g., soybean oil) having at least one saturated molecular marker and at least one unsaturated molecular marker can comprise high palmitic acid and high oleic acid content; high palmitic acid and low linoleic acid content; high palmitic acid and low linolenic acid content; high palmitic acid, high oleic acid, and low linoleic acid content; high palmitic acid, high oleic acid, and low linolenic acid content; high palmitic acid, high oleic acid, low linoleic acid, low linolenic acid content; or high palmitic acid, high stearic acid, high oleic acid, low linoleic acid, and low linolenic acid content, relative to a control plant, plant part, seed, or product (e.g., oil) without the saturated molecular marker(s) or the unsaturated molecular marker(s).

The saturated fatty acid content, palmitic acid content, stearic acid content, monounsaturated fatty acid content, oleic acid content, and/or saturated plus monounsaturated fatty acid content, expressed as percent of total fatty acids, in the soybean plants, soybean parts, soybean seeds, or soybean product (e.g., soybean oil) having the saturated and unsaturated markers can be greater than that of a control plant, plant part, seed, or product (e.g., oil) without the saturated or unsaturated markers, and the difference (by subtraction) can be about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%.

The polyunsaturated fatty acid content, linoleic acid content, and/or linolenic acid content, expressed as percent of total fatty acids, in the soybean plants, soybean parts, soybean seeds, or soybean product (e.g., soybean oil) having the saturated and unsaturated markers can be less than that of a control plant, plant part, seed, or product (e.g., oil) without the saturated or unsaturated markers, and the difference (by subtraction) can be about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 99%.

The saturated to unsaturated fatty acid composition and or saturated plus monounsaturated to polyunsaturated fatty acid composition, expressed as ratios, in the soybean plants, soybean parts, soybean seeds, or soybean product (e.g., soybean oil) having the saturated and unsaturated markers can be greater than those of a control plant, plant part, seed, or product (e.g., oil) without the saturated or unsaturated markers, and the difference (ratios in marker-positive soybean plants, soybean parts, soybean seeds, or soybean product (e.g., soybean oil)/ratios in a marker-negative control plant, plant part, plant seed, or plant oil) can be at least about 1%, 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 600%, 700%, 800%, 900%, or 1000%.

In certain embodiments, soybean plants, soybean parts, soybean seeds, or soybean product (e.g., soybean oil) having at least one saturated molecular marker and at least one unsaturated molecular marker disclosed herein can comprise a saturated fatty acid content of about 17.5% to about 35% (of total fatty acids) (e.g., about 17.5-20%, 20-22.5%, 22.5-25%, 25-27.5%, 27.5-30%, 30-32.5%, or 32.5-35%) and a polyunsaturated fatty acid content of about 5% to 30% (of total fatty acids) (e.g., about 15-17.5%, 17.5-20%, 20-22.5%, 22.5-25%, 25-27.5%, 27.5-30%) by weight. In certain embodiments, soybean plants, soybean parts, soybean seeds, or soybean product (e.g., soybean oil) having at least one saturated molecular marker and at least one unsaturated molecular marker disclosed herein can comprise a palmitic acid content of about 15% to about 30% (e.g., about 15-17.5%, 17.5-20%, 20-22.5%, 22.5-25%, 25-27.5%, 27.5-30%), a stearic acid content of about 2.5% to about 3.5% (e.g., about 2.5-3%, 3-3.5%), an oleic acid content of about 35% to about 80% (e.g., about 35-40%, 40-45%, 45-50%, 50-55%, 55-60%, 65-70%, 75-80%), a linoleic acid content of about 5% to 25% (e.g., about 5-10%, 10-15%, 15-20%, 20-25%), and/or a linolenic acid content of about 1% to about 5% (e.g., about 1-2%, 2-3%, 3-4%, 4-5%) by weight of total fatty acids. In particular embodiments, soybean plants, soybean parts, soybean seeds, or soybean product (e.g., soybean oil) having at least one saturated molecular marker and at least one unsaturated molecular marker disclosed herein can comprise fatty acid composition of about 90% saturated plus monounsaturated fatty acids and about 10% polyunsaturated fatty acids, similar to palm oil composition (i.e., 50% saturated fatty acids, approximately 40% monounsaturated fatty acids, and approximately 10% polyunsaturated fatty acids).

Soybean Oil and Soybean Oil Products Comprising High Saturated, High Monounsaturated, and/or Low Polyunsaturated Fatty Acid (HPHOLL) Content

Provided herein is soybean oil having an HPHOLL content. Such HPHOLL oil include soybean oil containing a saturated and/or unsaturated molecular marker disclosed herein, as well as soybean oil that do not contain a saturated or unsaturated molecular marker disclosed herein. The HPHOLL soybean oil of the present disclosure can be produced by any methods. For example, the HPHOLL soybean oil of the present disclosure can be produced, exclusively or nonexclusively, from plants or seeds that contain a saturated or unsaturated molecular marker disclosed herein, from plants or seeds that do not contain a saturated or unsaturated molecular marker disclosed herein, from plants or seeds produced or selected according to the methods provided herein, or from plants or seeds not produced or selected according to the methods provided herein.

The HPHOLL soybean oil provided herein can comprise: high palmitic acid content; high stearic acid content; high palmitic acid and high oleic acid content; high palmitic acid and low linoleic acid content; high palmitic acid and low linolenic acid content; high palmitic acid, high oleic acid, and low linoleic acid content; high palmitic acid, high oleic acid, and low linolenic acid content; high palmitic acid, high oleic acid, low linoleic acid, low linolenic acid content; or high palmitic acid, high stearic acid, high oleic acid, low linoleic acid, and low linolenic acid content, relative to oil produced from a reference soybean oil, e.g., produced from reference soybean plants or seeds. Reference soybean oil can be produced from commercially available or standard soybean plants or seeds, soybean plants or seeds produced without assaying for or selecting based on a saturated or unsaturated molecular marker disclosed herein, or soybean plants or seeds that do not contain the saturated or unsaturated molecular marker provided herein. The HPHOLL soybean oil of the present disclosure can have high saturated to unsaturated fatty acid composition relative to reference soybean oil. The soybean oil of the present disclosure can have high saturated plus monounsaturated to polyunsaturated fatty acid composition relative to reference soybean oil.

Palmitic, stearic and other saturated fatty acids are typically solid at room temperature, in contrast to the unsaturated fatty acids, which remain liquid. Because saturated fatty acids have no double bonds in the acyl chain, they remain stable to oxidation at elevated temperatures. Saturated fatty acids are important components in margarines and chocolate formulations, and for many food applications, increased levels of saturated fatty acids are desired. In some embodiments, the soybean oil provided herein can comprise saturated fatty acid of 30% (of total fatty acids) or about 30% to about 40% (of total fatty acids) by weight.

Oleic acid has one double bond, but is still relatively stable at high temperatures, and oils with high levels of oleic acid are suitable for cooking and other processes where heating is required. Recently, increased consumption of high oleic oils has been recommended, because oleic acid appears to lower blood levels of low density lipoproteins (“LDLs”) without affecting levels of high density lipoproteins (“HDLs”). However, some limitation of oleic acid levels is desirable, because when oleic acid is degraded at high temperatures, it creates negative flavor compounds and diminishes the positive flavors created by the oxidation of linoleic acid. Neff et al., JAOCS, 77 :1303-1313 (2000); Warner et al., J. Agric. Food Chem. 49:899-905 (2001). Preferred oils have oleic acid levels that are 65-85% or less by weight, in order to limit off-flavors in food applications such as frying oil and fried food. Other preferred oils have oleic acid levels that are greater than 55% by weight in order to improve oxidative stability.

Linoleic acid is a major polyunsaturated fatty acid in foods and is an essential nutrient for humans. It is a desirable component for many food applications because it is a major precursor of fried food flavor substances such as 2,4 decadienal, which make fried foods taste good. However, linoleic acid has limited stability when heated. Preferred food oils have linoleic acid levels that are 10% or greater by weight, to enhance the formation of desirable fried food flavor substances, and also are 25% or less by weight, so that the formation of off-flavors is reduced. Linoleic acid also has cholesterol-lowering properties, although dietary excess can reduce the ability of human cells to protect themselves from oxidative damage, thereby increasing the risk of cardiovascular disease. Toborek et al., Am J. Clin. J. 75:119-125 (2002). See generally Flavor Chemistry of Lipid Foods, editors D. B. Min & T. H. Smouse, Am Oil Chem. Soc., Champaign, Ill. (1989).

Linoleic acid, having a lower melting point than oleic acid, further contributes to improved cold flow properties desirable in biodiesel and biolubricant applications. Preferred oils for most applications have linoleic acid levels of 30% or less by weight, because the oxidation of linoleic acid limits the useful storage or use-time of frying oil, food, feed, fuel and lubricant products. See generally, Physical Properties of Fats, Oils, and Emulsifiers, ed. N. Widlak, AOCS Press (1999); Erhan & Asadauskas, Lubricant Basestocks from Vegetable Oils, Industrial Crops and Products, 11:277-282 (2000). In addition, high linoleic acid levels in cattle feed can lead to undesirably high levels of linoleic acid in the milk of dairy cattle, and therefore poor oxidative stability and flavor. Timmons et al., J. Dairy Sci. 84:2440-2449 (2001). A broadly useful oil composition has linoleic acid levels of 10-25% by weight.

Linolenic acid is also an important component of the human diet. It is used to synthesize the ω-3 family of long-chain fatty acids and the prostaglandins derived therefrom. However, its double bonds are highly susceptible to oxidation, so that oils with high levels of linolenic acid deteriorate rapidly on exposure to air, especially at high temperatures. Partial hydrogenation of such oils is often necessary before they can be used in food products to retard the formation of off-flavors and rancidity when the oil is heated, but hydrogenation creates unhealthy trans fatty acids which can contribute to cardiovascular disease. To achieve improved oxidative stability, and reduce the need to hydrogenate oil, preferred oils have linolenic acid levels that are 8% or less by weight, 6% or less, 4% or less, and more preferably 0.5-2% by weight of the total fatty acids in the oil of the present invention. Oil having low polyunsaturated fatty acid content provided herein is suited for the production of shortening, margarine and other semi-solid vegetable fats used in foodstuffs. Production of these fats typically involves hydrogenation of unsaturated oils such as soybean oil, corn oil, or canola oil. In contrast, HPHOLL oil provided herein has increased oxidative and flavor stability, without need for hydrogenation. Accordingly, the HPHOLL oil provided herein has reduced processing costs and reduced unhealthy trans isomers.

In some embodiments, the HPHOLL soybean oil provided herein can have about 4 percent more in palmitic acid or about 0.5 percent more stearic acid content compared to reference soybean oil. The soybean oil provided herein can comprise a saturated fatty acid content of about 17.5% to about 35% (of total fatty acids), or in some embodiments a saturated fatty acid content of about 30% to about 40% by weight. Soybean oil provided herein can comprise a palmitic acid content of about 15% to about 30% (of total fatty acids) and/or a stearic acid content of about 2.5% to about 3.5% (of total fatty acids). The soybean oil provided herein can comprise a saturated fatty acid content of about 17.5% to about 35% (of total fatty acids) and a polyunsaturated fatty acid content of about 5% to 30% (of total fatty acids). In some embodiments, the soybean oil provided herein can comprise about 25% to about 45% saturated fatty acids and about 55 to about 80% monounsaturated fatty acid; about 30% to about 40% palmitic acid and/or about 55 to about 80% oleic acid; or about 30% to about 40% palmitic acid and about 40 to about 75% oleic acid content. Additionally, oil provided herein can comprise about 15% to about 30% palmitic acid, about 2.5% to about 3.5% stearic acid, about 35% to about 80% oleic acid, a linoleic acid content of about 5% to 25%, and/or about 1% to about 5% linoleic acid content. In one embodiment, an oil of the present invention preferably has an oil composition that is about 25% to 45% palmitic acid, about 55% to 80% oleic acid, about 10 to 40% linoleic acid, and about 6% or less linolenic acid; more preferably has an oil composition that is 30% to 40% palmitic acid, about 40% to 75% oleic acid, about 10 to 18% linoleic acid, and about 4.5% or less linolenic acid.

In another embodiment, an oil of the present invention has an oil composition that is about 40-75% monounsaturates, about 30-40% saturates, and about 10-20% polyunsaturates. In another embodiment, an oil of the present invention has an oil composition that is about 40-75% monounsaturates, about 30-40% saturates, and about 10-25% polyunsaturates. In particular embodiments, oil of the present disclosure can comprise about 90% saturated plus monounsaturated fatty acids and about 10% polyunsaturated fatty acids, similar to palm oil composition (i.e., 50% saturated fatty acids, approximately 40% monounsaturated fatty acids, and approximately 10% polyunsaturated fatty acids).

In other embodiments, the percentage of palmitic acid is 15% or greater, 20% or greater, 25% or greater; 30% or greater; 35% or greater; 40% or greater; 45% or greater; or ranges from 15 to 45%; 15 to 30%; 25 to 45%; 30 to 45%; 35 to 45%; 40 to 45%; 25 to 30%; 30 to 35%; 35 to 40%; 40 to 45%; or greater than 45%. Suitable percentage ranges for palmitic acid content in oils of the present invention also include ranges in which the lower limit is selected from the following percentages: 25, 26, 27, 28, 29, 30, 31, 32, 33, 34, 35, 36, 37, 38, 39, 40, 41, 42, 43, 44, or 45 percent; and the upper limit is selected from the following percentages: 35, 36, 37, 38, 39, 40, 41, 42, 43, 44, or 45 percent.

In other embodiments, the percentage of oleic acid is 35% or greater; 40% or greater; 45% or greater; 50% or greater; 55% or greater; 60% or greater; 65% or greater; 70% or greater; 75% or greater; or 80% or greater; or is a range from 35 to 50%; 50 to 80%; 55 to 80%; 55 to 75%; 55 to 65%; 65 to 80%; 65 to 75%; 65 to 70%; 70 to 75%; or 75 to 80%. Suitable percentage ranges for oleic acid content in oils of the present invention also include ranges in which the lower limit is selected from the following percentages: 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 percent; and the upper limit is selected from the following percentages: 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 percent. In some embodiments, oil provided herein can comprise palmitic acid content of 15-45% and oleic acid content of 35-80%, for example, 15-30% palmitic acid and 65-80% oleic acid; 15-30% palmitic acid and 50-65% oleic acid; 15-30% palmitic acid and 35-50% oleic acid; 25-35% palmitic acid and 60-70% oleic acid; 25-35% palmitic acid and 50-60% oleic acid; 25-35% palmitic acid and 35-50% oleic acid; 35-40% palmitic acid and 55-60% oleic acid; 35-40% palmitic acid and 45-55% oleic acid; 35-40% palmitic acid and 35-45% oleic acid; 40-45% palmitic acid and 50-55% oleic acid; or 40-45% palmitic acid and 35-50% oleic acid.

In these other embodiments, the percentage of linoleic acid in oil provided herein is a range from 5 to 30%; 5 to 25%; 10 to 30%; 10 to 25%; 10 to 20%; 10 to 15%; 5 to 20%; 5 to 15%; 5 to 10%; 15 to 30%; 15 to 25%; 15 to 20%; 20 to 30%; or 25 to 30%. Suitable percentage ranges for linoleic acid content in oils of the present invention also include ranges in which the lower limit is selected from the following percentages: 5, 6, 7, 8, 9, 10, 11, 12, 13, 14, 15, 16, 17, 18, 19, 20, 21, 22, 23, 24, or 25 percent; and the upper limit is selected from the following percentages: 15, 16, 17, 18, 19, 20, 21, 22, 23, 24, 25, 26, 27, 28, 29, or 30 percent. In some embodiments, oil provided herein can comprise palmitic acid content of 15-45% and linoleic acid content of 5%-30%, for example, 15-30% palmitic acid and 15-30% linoleic acid; 15-30% palmitic acid and 10-15% linoleic acid; 15-30% palmitic acid and 5-10% linoleic acid; 25-35% palmitic acid and 15-30% linoleic acid; 25-35% palmitic acid and 10-15% linoleic acid; 25-35% palmitic acid and 5-10% linoleic acid; 35-40% palmitic acid and 15-30% linoleic acid; 35-40% palmitic acid and 10-15% linoleic acid; 35-40% palmitic acid and 5-10% linoleic acid; 40-45% palmitic acid and 15-30% linoleic acid; 40-45% palmitic acid and 10-15% linoleic acid; or 40-45% palmitic acid and 5-10% linoleic acid.

In these other embodiments, the percentage of linolenic acid in oil provided herein is 10% or less; 9% or less; 8% or less; 7% or less; 6% or less; 5% or less; 4.5% or less; 4% or less; 3.5% or less; 3% or less; 3.0% or less; 2% or less; or 1% or less; or is a range from 1 to 5%; 0.5 to 2%; 0.5 to 3%; 0.5 to 4.5%; 0.5% to 6%; 3 to 5%; 3 to 6%; 3 to 8%; 1 to 2%; 1 to 3%; or 1 to 4%. In some embodiments, oil provided herein can comprise palmitic acid content of 15-45% and linolenic acid content of 1-10%, for example, 15-30% palmitic acid and 5-10% linolenic acid; 15-30% palmitic acid and 3-5% linolenic acid; 15-30% palmitic acid and 1-3% linolenic acid; 25-35% palmitic acid and 5-10% linolenic acid; 25-35% palmitic acid and 3-5% linolenic acid; 25-35% palmitic acid and 1-3% linolenic acid; 35-40% palmitic acid and 5-10% linolenic acid; 35-40% palmitic acid and 3-5% linolenic acid; 35-40% palmitic acid and 1-3% linolenic acid; 40-45% palmitic acid and 5-10% linolenic acid; 40-45% palmitic acid and 3-5% linolenic acid; or 40-45% palmitic acid and 1-3% linolenic acid.

In some embodiments, HPHOLL soybean oil provided herein is produced from soybean plants and seeds that are produced according to the methods of the present disclosure and having an HPHOLL phenotype. For example, soybean oil provided herein can comprise at least one saturated quantitative trait locus (QTL) associated with high palmitic acid and/or high stearic acid content and/or at least one unsaturated QTL associated with high oleic acid, low linoleic acid, and/or low linolenic acid content. Such saturated and/or unsaturated QTL can comprise at least one SNP marker. The presence of an SNP marker in soybean oil can be detected by methods for detecting DNA fragments in samples, including PCR and quantitative real-time PCR, as described for example in Duan at al. 2021 Food Sci Biotechnol 30(1):129-135, the entirety of which is herein incorporated by reference.

In specific embodiments, soybean oil provided herein contains a saturated QTL, e.g., Gm08:063500, Gm08:045000, Gm08:057400, Gm08:072100, Gm08:083900, Gm08:084300, Gm08:092100 and/or Gm08:126400, and/or an unsaturated QTL, e.g., Gm10:50014440, Gm20:35318088, Gm14:45937922, Gm14:45937935, and/or Gm02:41422213. In some embodiments, the saturated SNP marker is a G or an A at position 4879302, a T or a C at position 3567986, a T or a C at position 4416970, an A or a T at position 5521970, an A or a T at position 6333332, a T or a C at position 6357981, a T or a C at position 6958927, an A or a G at position 9738629 of chromosome 8; and the unsaturated SNP marker is an A or a G at position 50014440 of chromosome 10; a G or a C at position 35318088 of chromosome 20; an A or a G at position 45937922 of chromosome 14, an A or a G at position 45937935 of chromosome 14, and/or an A or a G at position 41422213 of chromosome 2 of a genome the soybean plants or seeds. The G at position 4879302, the T at position 3567986, the T at position 4416970, the A at position 5521970, the A at position 6333332, the T at position 6357981, the T at position 6958927, or the A at position 9738629 of chromosome 8 can be associated with high palmitic acid content, and the A at position 50014440 of chromosome 10, the G at position 35318088 of chromosome 20, the A at position 45937922 of chromosome 14, the A at position 45937935 of chromosome 14, and/or the A at position 41422213 of chromosome 2 can be associated with high oleic acid, low linoleic acid, and/or low linolenic acid content.

In a preferred embodiment, the oil is a soybean oil. In many embodiments described herein, the soybean oil was extracted from the seed of a plant selected from the group consisting Glycine max, Glycine soja, Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine stenophita, Glycine tabacina, and Glycine tomentella. In one embodiment, the soybean oil was extracted from the seed of a Glycine max plant.

The oil of the present invention can be a blended oil, synthesized oil, or an oil generated from a seed having an appropriate oil composition. The oil can be a crude oil such as crude soybean oil, or can be a processed oil, for example the oil can be refined, bleached, deodorized, winterized, and/or modified. As used herein, “refining” refers to a process of treating natural or processed fat or oil to remove impurities, and may be accomplished by treating fat or oil with caustic soda, followed by centrifugation, washing with water, and heating under vacuum. “Bleaching” refers to a process of treating a fat or oil to remove or reduce the levels of coloring materials in the fat or oil. Bleaching may be accomplished by treating fat or oil with activated charcoal or Fullers (diatomaceous) earth. “Deodorizing” refers to a process of removing components from a fat or oil that contribute objectionable flavors or odors to the end product, and may be accomplished by use of high vacuum and superheated steam washing. “Winterizing” refers to a process of removing saturated glycerides from an oil, and may be accomplished by chilling and removal of solidified portions of fat from an oil. Modification can include epoxidation, alcoholysis, transesterification, direct esterification, metathesis, isomerization, monomer modification, and various forms of polymerization and copolymerization, including heat bodying.

An oil of the present invention is particularly suited to use as a cooking or frying oil. Because of its reduced polyunsaturated fatty acid content, the oil of the present invention does not require the extensive processing of typical oils because fewer objectionable odorous and colorant compounds are present. The present soybean oil may be advantageously used as a palm oil substitute because of its improved oxidative stability, the reduction of off-flavor precursors, and its high saturated fatty acid level.

Also provided herein are soybean oil products produced from HPHOLL soybean oil provided herein. Soybean oil have broad application in food products and industrial uses. Soybean oil products of the present disclosure include anti-static agents, caulking compounds, disinfectants, fungicides, inks, paints, protective coatings, wallboard, anti-foam agents, alcohol, margarine, paint, ink, rubber, shortening, cosmetics, and alkyd resins. Alkyd resins are dissolved in carrier solvents to make oil-based paints. The basic chemistry for converting vegetable oils into an alkyd resin under heat and pressure is well understood to those of skill in the art.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting. Unless otherwise noted, all parts and percentages are by dry weight.

### EXAMPLES

### Example 1: Identifying SNP Markers Associated with High-Palmitic Acid Phenotype in Soybean Seeds

The soybean line A28 has a high palmitic acid phenotype in that its seeds contain high palmitic acid levels, as verified by gas chromatography-mass spectrometry (GC-MS). The soybean lines 26H956 and 7Q90012 have a high oleic acid, low linolenic acid (high monounsaturated fatty acid and low polyunsaturated fatty acid) phenotype in that their seeds contain high oleic acid and low linolenic acid levels as verified by GC-MS. Twenty (20) seeds of each variety were sowed in greenhouse. Two crosses, A28×26H956 and A28×7Q90012 were made and 38 and 39 F1 seeds were harvested, respectively. All F1 seeds were selfed to produce 300 F2 seeds (A28×26H956) and 150 F2 seeds (A28×7Q90012), respectively. Three hundred (300) F2 seeds (A28×26H956) and 150 F2 seeds (A28×7Q90012) were tissue sampled and submitted for genome wide genotyping with approximately 1000 SNP markers.

After filtration for quality control, 641 SNPs with a minor allele frequency (MAF) 5% in 272 soybean lines were used for association mapping of palmitic acid and stearic acid using the Fixed and random model Circulating Probability Unification (FarmCPU) model. Three (3) SNPs for palmitic acid and 2 SNPs for stearic acid were identified as significant at the level of P<0.0001. One marker located at position 4879302 base pairs on chromosome 8, subsequently named Gm08:063500, was found significantly associated with elevated palmitic acid and stearic acid levels in both A28×26H956 and A28×7Q90012F2 populations. As shown in Table 1, plants with allele C had approximately 4-6% higher palmitic acid content in seeds as compared to plants with allele T. On the other hand, as shown in Table 2, plants with allele T had approximately 0.7-0.9% higher stearic acid content in seeds as compared to plants with allele C.

TABLE 1
Association of Gm08: 063500 with palmitic acid content
(% of total fatty acids) in soybean seeds

| Alleles | F2 (26H956/A28) | F2 (7Q90012/A28) |
|---|---|---|
| C | 21.9 | 19.4 |
| H | 17.1 | 16.0 |
| T | 16.0 | 15.3 |

TABLE 2
Association of Gm08: 063500 with stearic acid content
(% of total fatty acids) in soybean seeds

| Alleles | F2 (26H956/A28) | F2 (7Q90012/A28) |
|---|---|---|
| C | 2.5 | 2.5 |
| H | 3.1 | 3.0 |
| T | 3.4 | 3.2 |

In separate experiments, suitability of Gm08:063500 as a high palmitic acid marker was validated in the two different populations, A28×26H956 and A28×7Q90012F2. As shown in Table 3, positive (allele C) Gm08:063500 was associated with higher palmitic acid content in BC1 seeds as compared to BC1 seeds with negative (allele T) or heterozygous Gm08:063500.

TABLE 3
Association of Gm08: 063500 with average palmitic
acid content (% of total fatty acids) in BC1F2
26H956/A28 and 7Q90012/A28 soybean seeds

| | BC1F2 | BC1F2 |
|---|---|---|
| | (2*26H956/A28) | (2*7Q90012/A28) |
| Gm08: 063500 positive (C) | 18.3 | 19.6 |
| Heterozygous | 13.3 | 15.2 |
| Gm08: 063500 negative (T) | 11.4 | 12.9 |

### Example 2: Identifying Additional SNP Markers Associated with High Palmitic Acid Phenotype

Fine-mapping was conducted using eight markers between chr8:3,567,986 and chr8:9738629 shown in Table 4. As shown in Table 5, fine-mapping revealed that these markers are associated with increased palmitic acid content, with the marker Gm08:084300 showing the highest association with high palmitic acid content. These markers are associated with high palmitic acid and low stearic acid phenotype. There is an inverse correlation between palmitic and stearic acid content.

TABLE 4
High palmitic acid markers selected for fine mapping

| Marker | Position | Desired | Control | Gene ID |
|---|---|---|---|---|
| Gm08: 063500 | chr8: 4879302 | G | A | Glyma.08G063500 |
| Gm08: 045000 | chr8: 3567986 | T | C | Glyma.08G045000 |
| Gm08: 057400 | chr8: 4416970 | T | C | Glyma.08G057400 |
| Gm08: 072100 | chr8: 5521970 | A | T | Glyma.08G072100 |
| Gm08: 083900 | chr8: 6333332 | A | T | Glyma.08G083900 |
| Gm08: 084300 | chr8: 6357981 | T | C | Glyma.08G084300 |
| Gm08: 092100 | chr8: 6958927 | T | C | Glyma.08G092100 |
| Gm08: 126400 | chr8: 9738629 | A | G | Glyma.08G126400 |

TABLE 5
Fine mapping of high palmitic acid/high stearic acid
markers using a BC2F2 population of 26H956/A28

| | | Gm08: | Gm08: | Gm08: | Gm08: | Gm08: | Gm08: | Gm08: | Gm08: |
|---|---|---|---|---|---|---|---|---|---|
| | * | 063500 | 045000 | 057400 | 072100 | 083900 | 084300 | 092100 | 126400 |
| Palmitic | 2 | 17.0 | 16.3 | 16.5 | 17.1 | 18.0 | 21.0 | 20.4 | 17.3 |
| acid | 1 | 13.7 | 13.5 | 13.6 | 13.5 | 13.6 | 13.6 | 13.7 | 14.4 |
| (%) | 0 | 11.6 | 12.1 | 11.7 | 12.0 | 11.3 | 10.8 | 11.0 | 12.9 |
| Stearic | 2 | 2.5 | 2.6 | 2.6 | 2.5 | 2.4 | 2.3 | 2.4 | 2.6 |
| acid | 1 | 2.8 | 2.8 | 2.8 | 2.8 | 2.8 | 2.8 | 2.8 | 2.8 |
| (%) | 0 | 2.9 | 2.9 | 2.9 | 2.9 | 2.9 | 2.9 | 2.9 | 2.7 |

* 2 = marker present in two alleles; 1 = marker present in one allele; 0 = marker absent

### Example 3: Selecting Plants with High Saturated Fatty Acid/High Monounsaturated Fatty Acid/Low Polyunsaturated Fatty Acid Content

### (1) Selection Based on Genotypic Markers

Markers Gm08:063500, Gm10:50014440, Gm20:35318088, Gm14:45937922, Gm14:45937935, and/or Gm02:41422213 were used to select soybean lines having a high saturated fatty acid (e.g., palmitic acid), high monounsaturated fatty acid (e.g., oleic acid), and low polyunsaturated fatty acid (e.g., linoleic acid, linolenic acid) (“HPHOLL”) phenotype in seeds. The marker characteristics are set forth in Table 6. As shown in Tables 7 and 8, selecting for Gm08:063500 identified plants with high palmitic acid content (e.g., the A28 line). Selecting for Gm10:50014440, Gm20:35318088, and Gm14:45937922 identified plants with a high monounsaturated fatty acid (e.g., oleic acid) and low polyunsaturated fatty acid (e.g., linoleic acid, linolenic acid) (HOLL) content (e.g., the 26H956 line). Selecting for Gm10:50014440, Gm20:35318088, Gm14:45937935, and Gm02:41422213 also identified plants with a high monounsaturated fatty acid and low polyunsaturated fatty acid (HOLL) content (e.g., 7Q90012).

Selecting for Gm08:063500 and one or more of Gm10:50014440, Gm20:35318088, Gm14:45937922, Gm14:45937935, and Gm02:41422213 resulted in a line with a high saturated fatty acid (e.g., palmitic acid), high monounsaturated fatty acid (e.g., oleic acid), and low polyunsaturated fatty acid (e.g., linoleic acid, linolenic acid) (TIPHOLL) content.

TABLE 6
Genotypic markers used for selecting HPHOLL plants

| Marker | Location | Position | Desired | Control | Gene ID |
|---|---|---|---|---|---|
| Gm08: 063500 | chr08: 4876657- | chr8: 4879302 | G | A | Glyma.08G063500 |
| | 4882865 | | | | |
| Gm10: 50014440 | chr10: 50013483- | chr10: 50014440 | A | G | Glyma.10G278000 |
| | 50015460 | | | | |
| Gm20: 35318088 | chr20: 35315629- | chr10: 35318088 | G | C | Glyma.20G111000 |
| | 35319063 | | | | |
| Gm14: 45937922 | chr14: 45935667- | chr14: 45937922 | A | G | Glyma.14G194300 |
| | 45939896 | | | | |
| Gm14: 45937935 | chr14: 45935667- | chr14: 45937935 | A | G | Glyma.14G194300 |
| | 45939896 | | | | |
| Gm02: 41422213 | chr02: 41419655- | chr02: 41422213 | A | G | Glyma.02G227200 |
| | 41423881 | | | | |

TABLE 7
HPHOLL plant selection based on genotypic markers from population 1

| | Genotype - | | | | | |
|---|---|---|---|---|---|---|
| | presence (2) and | | | | | |
| | absence (0) of | | | | | |
| | Gm08: 063500, | | | | | |
| | Gm10: 50014440, | | | | | |
| | Gm20: 35318088, | 16:0 | 18:0 | 18:1 | 18:2 | 18:3 |
| Line Name | Gm14: 45937922 | Palmitic | Stearic | Oleic | Linoleic | Linolenic |
| HOLL- 26H956 | 0222 | 8.3 | 2.2 | 85.3 | 1.4 | 2.7 |
| A28 | 2000 | 30.2 | 2.8 | 9.7 | 45.0 | 12.4 |
| Lines in the population | 0022 | 15.1 | 2.9 | 40.8 | 37.3 | 3.9 |
| Lines in the population | 0220 | 10.9 | 2.8 | 80.0 | 2.7 | 3.4 |
| Lines in the population | 0222 | 10.6 | 2.6 | 80.2 | 3.8 | 2.8 |
| Lines in the population | 2022 | 22.2 | 2.6 | 35.2 | 36.0 | 4.1 |
| Lines in the population | 2220 | 17.5 | 2.6 | 72.8 | 2.8 | 4.3 |
| Lines in the population | 2222 | 17.1 | 2.5 | 72.5 | 4.5 | 3.4 |

TABLE 8
HPHOLL plant selection based on genotypic markers from population 2

| | Genotype - | | | | | |
|---|---|---|---|---|---|---|
| | presence (2) and | | | | | |
| | absence (0) of | | | | | |
| | Gm08: 063500, | | | | | |
| | Gm10: 50014440, | | | | | |
| | Gm20: 35318088, | | | | | |
| | Gm14: 45937935, | 16:0 | 18:0 | 18:1 | 18:2 | 18:3 |
| Line Name | Gm02: 41422213 | Palmitic | Stearic | Oleic | Linoleic | Linolenic |
| HOLL_7Q90012 | 02222 | 6.7 | 3.0 | 84.2 | 5.0 | 1.0 |
| A28 | 20000 | 29.4 | 3.1 | 10.4 | 45.8 | 11.3 |
| Lines in the population | 00022 | 15.3 | 3.8 | 18.4 | 60.8 | 1.6 |
| Lines in the population | 00222 | 13.6 | 3.6 | 27.1 | 54.2 | 1.5 |
| Lines in the population | 02002 | 11.9 | 3.4 | 37.5 | 41.3 | 5.9 |
| Lines in the population | 02022 | 14.6 | 4.0 | 26.7 | 53.1 | 1.6 |
| Lines in the population | 02202 | 10.3 | 2.7 | 73.5 | 8.7 | 4.9 |
| Lines in the population | 02222 | 10.5 | 3.1 | 66.0 | 18.4 | 1.9 |
| Lines in the population | 20000 | 11.2 | 3.7 | 15.7 | 58.4 | 11.0 |
| Lines in the population | 20022 | 22.5 | 3.2 | 17.1 | 55.5 | 1.7 |
| Lines in the population | 20222 | 19.7 | 3.4 | 27.5 | 47.7 | 1.7 |
| Lines in the population | 22002 | 23.7 | 3.0 | 21.1 | 44.9 | 7.3 |
| Lines in the population | 22022 | 18.5 | 3.2 | 26.5 | 49.8 | 2.0 |
| Lines in the population | 22202 | 18.1 | 2.7 | 34.3 | 41.1 | 3.8 |
| Lines in the population | 22222 | 17.9 | 2.7 | 58.6 | 18.5 | 2.4 |

### (2) Phenotypic Selection of HPHOLL Back-Crossed Soybean Seeds

Among the 26H956/A28 and 7Q90012/A28 backcross (BC1) population, different types of HPHOLL lines having different oil compositions were identified and selected by using the Gm08:063500 marker and one or more unsaturated markers (Gm10:50014440, Gm20:35318088, Gm14:45937922, Gm14:45937935, and Gm02:41422213). The fatty acid profiles in BC1 seeds from the 26H956/A28 and 7Q90012/A28 plant population are shown in Tables 9 and 10. Based on the industry preference, products having a desired HPHOLL oil composition tailored to the preference and needs of respective industry can be produced and advanced among different soybean products comprising different HPHOLL oil compositions. For example, Bin 2 was selected among the 10 types of HPHOLL soybean lines in Table 9 according to a particular food science industry preference. Plant 4 was selected among the 7 types of HPHOLL soybean lines in Table 10 according to a particular food science industry preference.

TABLE 9
Oil composition in BC1 seeds (26H956/A28)

| | | 16:0 | 18:0 | 18:1 | 18:2 | 18:3 |
|---|---|---|---|---|---|---|
| Line Name | Genotype | Palmitic | Stearic | Oleic | Linoleic | Linolenic |
| A28 | Gm08: 063500 | 30.00 | 3.00 | 9.10 | 47.20 | 10.70 |
| HOLL- 26H956 | Gm10: 50014440, | 8.30 | 2.20 | 85.30 | 1.50 | 2.70 |
| | Gm20: 35318088, | | | | | |
| | Gm14: 45937922 | | | | | |
| Bin1 | Gm08: 063500, | 21.56 | 2.42 | 61.54 | 9.97 | 4.51 |
| Bin2 | Gm10: 50014440, | 20.06 | 2.46 | 66.62 | 6.77 | 4.08 |
| Bin3 | Gm20: 35318088, | 19.35 | 2.40 | 70.30 | 4.29 | 3.65 |
| Bin4 | Gm14: 45937922 | 18.71 | 2.43 | 72.02 | 3.39 | 3.45 |
| Bin5 | | 18.22 | 2.54 | 72.64 | 3.21 | 3.40 |
| Bin6 | | 17.66 | 2.44 | 72.65 | 4.14 | 3.11 |
| Bin7 | | 16.88 | 2.51 | 75.26 | 2.36 | 2.99 |
| Bin8 | | 15.52 | 2.38 | 74.04 | 4.94 | 3.13 |
| Bin9 | | 12.72 | 2.79 | 77.72 | 3.95 | 2.82 |
| Bin10 | | 10.91 | 2.48 | 81.92 | 2.13 | 2.56 |

TABLE 10
Oil composition in BC1 seeds (7Q90012/A28)

| | | 16:0 | 18:0 | 18:1 | 18:2 | 18:3 |
|---|---|---|---|---|---|---|
| Line Name | Genotype | Palmitic | Stearic | Oleic | Linoleic | Linolenic |
| A28 | Gm08: 063500 | 29.4 | 3.1 | 10.4 | 45.8 | 11.3 |
| HOLL2- | Gm10: 50014440, | 6.7 | 3.0 | 84.2 | 5.0 | 1.0 |
| 7Q90012 | Gm20: 35318088, | | | | | |
| | Gm14: 45937935, | | | | | |
| | Gm02: 41422213 | | | | | |
| Plant #1 | Gm08: 063500, | 21.7 | 2.7 | 25.4 | 45.5 | 4.7 |
| | Gm10: 50014440, | | | | | |
| | Gm20: 35318088, | | | | | |
| | Gm14: 45937935, | | | | | |
| | Gm02: 41422213 | | | | | |
| Plant #2 | Gm08: 063500, | 20.1 | 2.4 | 50.2 | 25.1 | 2.2 |
| Plant #3 | Gm10: 50014440, | 19.6 | 2.8 | 60.0 | 15.9 | 1.6 |
| Plant #4 | Gm20: 35318088, | 19.0 | 2.3 | 67.7 | 9.4 | 1.5 |
| Plant #5 | Gm14: 45937922 | 17.8 | 3.1 | 63.7 | 14.1 | 1.3 |
| Plant #6 | | 15.1 | 2.9 | 72.3 | 8.3 | 1.3 |
| Plant #7 | | 11.8 | 2.7 | 70.7 | 11.0 | 3.8 |

TABLE 11
Sequence Descriptions

| SEQ ID | |
|---|---|
| NO: | Description |
| 1 | Desired (high palmitic acid) sequence probe for Gm08: 063500 (chr8: 4879302 in |
| | Glyma.08G063500) |
| 2 | Undesired (normal or low palmitic acid) sequence probe for Gm08: 063500 (chr8: 4879302 in |
| | Glyma.08G063500) |
| 3 | Forward primer sequence for detecting Gm08: 063500 (chr8: 4879302 in Glyma.08G063500) |
| 4 | Reverse primer sequence for detecting Gm08: 063500 (chr8: 4879302 in Glyma.08G063500) |
| 5 | Desired (high palmitic acid) sequence probe for Gm08: 045000 (chr8: 3567986 in |
| | Glyma.08G045000) |
| 6 | Undesired (normal or low palmitic acid) sequence probe for Gm08: 045000 (chr8: 3567986 in |
| | Glyma.08G045000) |
| 7 | Forward primer sequence for detecting Gm08: 045000 (chr8: 3567986 in Glyma.08G045000) |
| 8 | Reverse primer sequence for detecting Gm08: 045000 (chr8: 3567986 in Glyma.08G045000) |
| 9 | Desired (high palmitic acid) sequence probe for Gm08: 057400 (chr8: 4416970 in |
| | Glyma.08G057400) |
| 10 | Undesired (normal or low palmitic acid) sequence probe for Gm08: 057400 (chr8: 4416970 in |
| | Glyma.08G057400) |
| 11 | Forward primer sequence for detecting Gm08: 057400 (chr8: 4416970 in Glyma.08G057400) |
| 12 | Reverse primer sequence for detecting Gm08: 057400 (chr8: 4416970 in Glyma.08G057400) |
| 13 | Desired (high palmitic acid) sequence probe for Gm08: 072100 (chr8: 5521970 in |
| | Glyma.08G072100) |
| 14 | Undesired (normal or low palmitic acid) sequence probe for Gm08: 072100 (chr8: 5521970 in |
| | Glyma.08G072100) |
| 15 | Forward primer sequence for detecting Gm08: 072100 (chr8: 5521970 in Glyma.08G072100) |
| 16 | Reverse primer sequence for detecting Gm08: 072100 (chr8: 5521970 in Glyma.08G072100) |
| 17 | Desired (high palmitic acid) sequence probe for Gm08: 083900 (chr8: 6333332 in |
| | Glyma.08G083900) |
| 18 | Undesired (normal or low palmitic acid) sequence probe for Gm08: 083900 (chr8: 6333332 in |
| | Glyma.08G083900) |
| 19 | Forward primer sequence for detecting Gm08: 083900 (chr8: 6333332 in Glyma.08G083900) |
| 20 | Reverse primer sequence for detecting Gm08: 083900 (chr8: 6333332 in Glyma.08G083900) |
| 21 | Desired (high palmitic acid) sequence probe for Gm08: 084300 (chr8: 6357981 in |
| | Glyma.08G084300) |
| 22 | Undesired (normal or low palmitic acid) sequence probe for Gm08: 084300 (chr8: 6357981 in |
| | Glyma.08G084300) |
| 23 | Forward primer sequence for detecting Gm08: 084300 (chr8: 6357981 in Glyma.08G084300) |
| 24 | Reverse primer sequence for detecting Gm08: 084300 (chr8: 6357981 in Glyma.08G084300) |
| 25 | Desired (high palmitic acid) sequence probe for Gm08: 092100 (chr8: 6958927 in |
| | Glyma.08G092100) |
| 26 | Undesired (normal or low palmitic acid) sequence probe for Gm08: 092100 (chr8: 6958927 in |
| | Glyma.08G092100) |
| 27 | Forward primer sequence for detecting Gm08: 092100 (chr8: 6958927 in Glyma.08G092100) |
| 28 | Reverse primer sequence for detecting Gm08: 092100 (chr8: 6958927 in Glyma.08G092100) |
| 29 | Desired (high palmitic acid) sequence probe for Gm08: 126400 (chr8: 9738629 in |
| | Glyma.08G126400) |
| 30 | Undesired (normal or low palmitic acid) sequence probe for Gm08: 126400 (chr8: 9738629 in |
| | Glyma.08G126400) |
| 31 | Forward primer sequence for detecting Gm08: 126400 (chr8: 9738629 in Glyma.08G126400) |
| 32 | Reverse primer sequence for detecting Gm08: 126400 (chr8: 9738629 in Glyma.08G126400) |
| 33 | Desired (high oleic, low linoleic/linolenic acids) sequence probe for Gm10: 50014440 |
| | (chr10: 50014440 in Glyma.10G278000) |
| 34 | Undesired (normal or low oleic, normal or high linoleic/linolenic acids) sequence probe for |
| | Gm10: 50014440 (chr10: 50014440 in Glyma.10G278000) |
| 35 | Forward primer sequence for detecting Gm10: 50014440 (chr10: 50014440 in |
| | Glyma.10G278000) |
| 36 | Reverse primer sequence for detecting Gm10: 50014440 (chr10: 50014440 in |
| | Glyma.10G278000) |
| 37 | Desired (high oleic, low linoleic/linolenic acids) sequence probe for Gm20: 35318088 |
| | (chr20: 35318088 in Glyma.20G111000) |
| 38 | Undesired (normal or low oleic, normal or high linoleic/linolenic acids) sequence probe for |
| | Gm20: 35318088 (chr20: 35318088 in Glyma.20G111000) |
| 39 | Forward primer sequence for detecting Gm20: 35318088 (chr20: 35318088 in |
| | Glyma.20G111000) |
| 40 | Reverse primer sequence for detecting Gm20: 35318088 (chr20: 35318088 in |
| | Glyma.20G111000) |
| 41 | Desired (high oleic, low linoleic/linolenic acids) sequence probe for Gm14: 45937922 |
| | (chr14: 45937922 in Glyma.14G194300) |
| 42 | Undesired (normal or low oleic, normal or high linoleic/linolenic acids) sequence probe for |
| | Gm14: 45937922 (chr14: 45937922 in Glyma.14G194300) |
| 43 | Forward primer sequence for detecting Gm14: 45937922 (chr14: 45937922 in |
| | Glyma.14G194300) |
| 44 | Reverse primer sequence for detecting Gm14: 45937922 (chr14: 45937922 in |
| | Glyma.14G194300) |
| 45 | Desired (high oleic, low linoleic/linolenic acids) sequence probe for Gm14: 45937935 |
| | (chr14: 45937935 in Glyma.14G194300) |
| 46 | Undesired (normal or low oleic, normal or high linoleic/linolenic acids) sequence probe for |
| | Gm14: 45937935 (chr14: 45937935 in Glyma.14G194300) |
| 47 | Forward primer sequence for detecting Gm14: 45937935 (chr14: 45937935 in |
| | Glyma.14G194300) |
| 48 | Reverse primer sequence for detecting Gm14: 45937935 (chr14: 45937935 in |
| | Glyma.14G194300) |
| 49 | Desired (high oleic, low linoleic/linolenic acids) sequence probe for Gm02: 41422213 |
| | (chr02: 41422213 in Glyma.02G227200) |
| 50 | Undesired (normal or low oleic, normal or high linoleic/linolenic acids) sequence probe for |
| | Gm02: 41422213 (chr02: 41422213 in Glyma.02G227200) |
| 51 | Forward primer sequence for detecting Gm02: 41422213 (chr02: 41422213 in |
| | Glyma.02G227200) |
| 52 | Reverse primer sequence for detecting Gm02: 41422213 (chr02: 41422213 in |
| | Glyma.02G227200) |

1. A method of producing a population of soybean plants or seeds comprising high palmitic acid or high stearic acid content relative to a control plant or seed, said method comprising:
(a) genotyping a first population of soybean plants or seeds for the presence of at least one saturated marker associated with high palmitic acid or high stearic acid content, wherein the at least one saturated marker is within 20 centimorgans of at least one saturated quantitative trait locus (QTL) associated with high palmitic acid and/or high stearic acid content located within a genomic region 3567986-9738629 of chromosome 8 of a soybean genome;
(b) selecting from the first population one or more soybean plants or seeds comprising one or more alleles comprising said at least one saturated marker associated with high palmitic acid or high stearic acid content; and
(c) producing a second population of progeny soybean plants or seeds from the one or more soybean plants or soybean seeds selected from the first population,
wherein the second population of progeny soybean plants or seeds comprises one or more alleles comprising said at least one saturated marker, and
wherein the second population of progeny soybean plants or seeds comprises high palmitic acid or high stearic acid content relative to a control population.

2. The method of claim 1, wherein the at least one saturated QTL associated with high palmitic acid or high stearic acid content is one or more of Gm08:063500, Gm08:045000, Gm08:057400, Gm08:072100, Gm08:083900, Gm08:084300, Gm08:092100, and Gm08:126400.

3. The method of claim 1 or 2, wherein said at least one saturated QTL comprises at least one single nucleotide polymorphisms (SNP), and the at least one saturated marker comprises an allele of the at least one SNP.

4. The method of claim 3, wherein the at least one SNP is a G or an A at position 4879302, a T or a C at position 3567986, a T or a C at position 4416970, an A or a T at position 5521970, an A or a T at position 6333332, a T or a C at position 6357981, a T or a C at position 6958927, and/or an A or a G at position 9738629 of chromosome 8 the soybean genome,
wherein the G at position 4879302, the T at position 3567986, the T at position 4416970, the A at position 5521970, the A at position 6333332, the T at position 6357981, the T at position 6958927, or the A at position 9738629 of chromosome 8 of the soybean genome is associated with high palmitic acid content.

5. The method of claim 4, wherein the at least one SNP is a G or an A at position 4879302 of chromosome 8 and/or a T or a C at position 6357981 of chromosome 8 of the soybean genome,
wherein the G at position 4879302 of chromosome 8 or the T at position 6357981 of chromosome 8 is associated with high palmitic acid content.

6. The method of any one of claims 3-5, wherein the genotyping comprises analyzing the at least one SNP or a haplotype.

7. The method of claim 6, wherein the genotyping comprises analyzing the at least one SNP or the haplotype using an oligonucleotide probe comprising at least 15 nucleotides, wherein the oligonucleotide probe has at least 90% sequence identity to a sequence of the same number of contiguous nucleotides of a sense or antisense DNA strand in a region comprising or adjacent to the at least one SNP in the soybean genome.

8. The method of claim 7, wherein the oligonucleotide probe comprises a nucleic acid sequence of any one of SEQ ID NOs: 1, 2, 5, 6, 9, 10, 13, 14, 17, 18, 21, 22, 25, 26, 29, and 30; or a nucleic acid sequence complementary to a nucleic acid sequence of any one of SEQ ID NOs: 1, 2, 5, 6, 9, 10, 13, 14, 17, 18, 21, 22, 25, 26, 29, and 30.

9. The method of claim 6, wherein the genotyping comprises analyzing the at least one SNP or the haplotype using a first primer and a second primer each comprising at least 15 nucleotides, wherein the first primer has at least 90% sequence identity to a sequence of the same number of contiguous nucleotides of a sense DNA strand of a region comprising or adjacent to the at least one SNP, and the second primer has at least 90% sequence identity to a sequence of the same number of contiguous nucleotides of an antisense DNA strand of the region comprising or adjacent to the at least one SNP.

10. The method of claim 9, wherein the first and second primers comprise any one pair of:
(i) nucleic acid sequences of SEQ ID NOs: 3 and 4;
(ii) nucleic acid sequences of SEQ ID NOs: 7 and 8;
(iii) nucleic acid sequences of SEQ ID NOs: 11 and 12;
(iv) nucleic acid sequences of SEQ ID NOs: 15 and 16;
(v) nucleic acid sequences of SEQ ID NOs: 19 and 20;
(vi) nucleic acid sequences of SEQ ID NOs: 23 and 24;
(vii) nucleic acid sequences of SEQ ID NOs: 27 and 28; and
(viii) nucleic acid sequences of SEQ ID NOs: 31 and 32.

11. The method of any one of claims 1-10, wherein the second population of progeny soybean plants or seeds comprises at least about 4% increase in palmitic acid content or at least about 0.5% increase in stearic acid content compared to a control population of soybean plants or seeds.

12. The method of any one of claims 1-11, wherein the second population of progeny soybean plants or seeds comprises oil having a palmitic acid content of about 15% to about 30% or a stearic acid content of about 2.5% to about 3.5%.

13. The method of any one of claims 1-12, comprising:
(a) genotyping the first population of soybean plants or seeds for the presence of (i) said at least one saturated marker associated with high palmitic acid or high stearic acid content and (ii) at least one unsaturated marker associated with high oleic acid, low linoleic acid, and/or low linolenic acid content, wherein the at least one unsaturated marker is within 20 centimorgans of at least one unsaturated QTL associated with high oleic acid, low linoleic acid, and/or low linolenic acid content;
(b) selecting from the first population one or more soybean plants or seeds comprising one or more alleles comprising (i) the at least one saturated marker associated with high palmitic acid and/or high stearic acid content and (ii) the at least one unsaturated marker associated with high oleic acid, low linoleic acid, and/or low linolenic acid content; and
(c) producing a second population of progeny soybean plants or seeds from the one or more soybean plants or soybean seeds selected from the first population,
wherein the second population of progeny soybean plants or seeds comprises one or more alleles comprising (i) said at least one saturated marker associated with high palmitic acid and/or high stearic acid content and (ii) said at least one unsaturated marker associated with high oleic acid, low linoleic acid, and/or low linolenic acid content,
wherein the second population of progeny soybean plants or seeds comprises high palmitic acid, high oleic acid, low linoleic acid, and/or low linolenic acid content relative to a control population.

14. The method of claim 13, wherein said at least one unsaturated QTL associated with high oleic acid, low linoleic acid, and/or low linolenic acid content is Gm10:50014440, Gm20:35318088, Gm14:45937922, Gm14:45937935, and/or Gm02:41422213.

15. The method of claim 13 or 14, wherein said at least one unsaturated marker associated with high oleic acid, low linoleic acid, and/or low linolenic acid content is located in Glyma.10G278000, Glyma.20G111000, Glyma.14G194300, and/or Glyma.02G227200 of the soybean genome.

16. The method of any one of claims 13-15, wherein said at least one unsaturated QTL comprises at least one SNP, and the at least one saturated marker comprises an allele of the at least one SNP.

17. The method of claim 15, wherein the at least one SNP is an A or a G at position 50014440 of chromosome 10, a G or a C at position 35318088 of chromosome 20, an A or a G at position 45937922 of chromosome 14, an A or a G at position 45937935 of chromosome 14, and/or an A or a G at position 41422213 of chromosome 2 of the soybean genome,
wherein the A at position 50014440 of chromosome 10, the G at position 35318088 of chromosome 20, the A at position 45937922 of chromosome 14, the A at position 45937935 of chromosome 14, and/or the A at position 41422213 of chromosome 2 is associated with high oleic acid, low linoleic acid, and/or low linolenic acid content.

18. The method of claim 16 or 17, wherein the genotyping comprises analyzing the at least one SNP or a haplotype using an oligonucleotide probe comprising a nucleic acid sequence of any one of SEQ ID NOs: 33, 34, 37, 38, 41, 42, 45, 46, 49, and 50; or a nucleic acid sequence complementary to a nucleic acid sequence of any one of SEQ ID NOs: 33, 34, 37, 38, 41, 42, 45, 46, 49, and 50.

19. The method of claim 16 or 17, wherein the genotyping comprises analyzing the at least one SNP or a haplotype using a first primer and a second primer comprising any one pair of:
(i) nucleic acid sequences of SEQ ID NOs: 35 and 36;
(ii) nucleic acid sequences of SEQ ID NOs: 39 and 40;
(iii) nucleic acid sequences of SEQ ID NOs: 43 and 44;
(iv) nucleic acid sequences of SEQ ID NOs: 47 and 48; and
(v) nucleic acid sequences of SEQ ID NOs: 51 and 52.

20. The method of any one of claims 13-19, wherein the second population of progeny soybean plants or seeds comprises oil comprising:
high palmitic acid and high oleic acid content;
high palmitic acid and low linoleic acid content;
high palmitic acid and low linolenic acid content;
high palmitic acid, high oleic acid, and low linoleic acid content;
high palmitic acid, high oleic acid, and low linolenic acid content;
high palmitic acid, high oleic acid, low linoleic acid, low linolenic acid content; or
high stearic acid, high oleic acid, low linoleic acid, and low linolenic acid content;
relative to a control population of soybean plants or seeds.

21. The method of claim 20, wherein the second population of progeny soybean plants or seeds comprises oil comprising high saturated fatty acid to unsaturated fatty acid composition relative to a control population of soybean plants or seeds.

22. The method of claim 20, wherein the second population of progeny soybean plants or seeds comprises oil comprising high saturated plus monounsaturated fatty acids to polyunsaturated fatty acid composition relative to a control population of soybean plants or seeds.

23. The method of any one of claims 13-22, wherein the second population of progeny soybean plants or seeds comprises oil having a palmitic acid content of about 15% to about 30%, an oleic acid content of about 35% to about 80%, a linoleic acid content of about 5% to 25%, and/or a linolenic acid content of about 1% to about 5%.

24. A method of introgressing a quantitative trait locus (QTL) associated with high palmitic acid and/or high stearic acid content, the method comprising:
(a) crossing a first soybean plant comprising a saturated QTL associated with high palmitic acid or high stearic acid content with a second soybean plant of a different genotype to produce one or more progeny plants or seeds; and
(b) selecting a progeny plant or seed comprising an allele comprising a polymorphic locus associated with said saturated QTL,
wherein the polymorphic locus is a chromosomal segment comprising a saturated marker within a genomic region 3567986-9738629 of chromosome 8 of a soybean genome.

25. The method of claim 24, wherein the saturated QTL is Gm08:063500, Gm08:045000, Gm08:057400, Gm08:072100, Gm08:083900, Gm08:084300, Gm08:092100 or Gm08:126400.

26. The method of claim 24 or 25, wherein said polymorphic locus comprises at least one single nucleotide polymorphisms (SNP), and the saturated marker comprises the at least one SNP.

27. The method of claim 26, wherein the at least one SNP is a G or an A at position 4879302, a T or a C at position 3567986, a T or a C at position 4416970, an A or a T at position 5521970, an A or a T at position 6333332, a T or a C at position 6357981, a T or a C at position 6958927, and/or an A or a G at position 9738629 of chromosome 8 the soybean genome,
wherein the G at position 4879302, the T at position 3567986, the T at position 4416970, the A at position 5521970, the A at position 6333332, the T at position 6357981, the T at position 6958927, or the A at position 9738629 of chromosome 8 of the soybean genome is associated with high palmitic acid content.

28. The method of claim 27, wherein the at least one SNP is a G or an A at position 4879302 of chromosome 8 and/or a T or an C at position 6357981 of chromosome 8 of a genome the soybean plants or seeds,
wherein the G at position 4879302 of chromosome 8 or the T at position 6357981 of chromosome 8 is associated with high palmitic acid content.

29. The method of any one of 24-28, comprising:
(a) crossing a first soybean plant comprising (i) the saturated QTL associated with high palmitic acid or high stearic acid content and (ii) an unsaturated QTL associated with high oleic acid, low linoleic acid, and/or low linolenic acid content with a second soybean plant of a different genotype to produce one or more progeny plants or seeds; and
(b) selecting a progeny plant or seed comprising an allele comprising a polymorphic locus associated with said saturated QTL and a polymorphic locus associated with said unsaturated QTL,
wherein the polymorphic locus linked to said unsaturated QTL is a chromosomal segment comprising an unsaturated marker within a genomic region 50013483-50015460 of chromosome 10, a genomic region 35315629-35319063 of chromosome 20, a genomic region 45935667-45939896 of chromosome 14, or a genomic region 41419655-41423881 of chromosome 2 of a soybean genome.

30. The method of claim 29, wherein the unsaturated QTL associated with high oleic acid, high linoleic acid, and/or low linolenic acid content is Gm10:50014440, Gm20:35318088, Gm14:45937922, Gm14:45937935, and/or Gm02:41422213.

31. The method of claim 29 or 30, wherein said polymorphic locus associated with said unsaturated QTL comprises at least one SNP, and said unsaturated marker comprises the at least one SNP.

32. The method of claim 31, wherein the at least one SNP is an A or a G at position 50014440 of chromosome 10, a G or a C at position 35318088 of chromosome 20, an A or a G at position 45937922 of chromosome 14, an A or a G at position 45937935 of chromosome 14, and/or an A or a G at position 41422213 of chromosome 2 of the soybean genome,
wherein the A at position 50014440 of chromosome 10, the G at position 35318088 of chromosome 20, the A at position 45937922 of chromosome 14, the A at position 45937935 of chromosome 14, and/or the A at position 41422213 of chromosome 2 is associated with high oleic acid, low linoleic acid, and/or low linolenic acid content.

33. The method of any one of claims 1-32, wherein the soybean plant or seed is selected from the group consisting of Glycine max, Glycine soja, Glycine arenaria, Glycine argyrea, Glycine canescens, Glycine clandestine, Glycine curvata, Glycine cyrtoloba, Glycine falcate, Glycine latifolia, Glycine latrobeana, Glycine max, Glycine microphylla, Glycine pescadrensis, Glycine pindanica, Glycine rubiginosa, Glycine soja, Glycine stenophita, Glycine tabacina, and Glycine tomentella.

34. A population of soybean plants or seeds produced by the method of any one of claims 1-33, comprising high palmitic acid, high oleic acid, high linoleic acid, and/or low linolenic acid content relative to a control population of soybean plants or seeds.

35. The population of soybean plants or seeds of claim 34, wherein the population comprises said saturated QTL associated with high palmitic acid or high stearic acid content, and/or an unsaturated QTL associated with high oleic acid, high linoleic acid, and/or low linolenic acid content at a greater frequency relative to a control population of soybean plants or seeds.

36. Oil produced from a population of soybean plants or seeds produced by the method of any one of claims 1-33, or from the population of soybean plants or seeds of claim 34 or 35, wherein the oil comprises:
high palmitic acid content;
high stearic acid content;
high palmitic acid and high oleic acid content;
high palmitic acid and low linoleic acid content;
high palmitic acid and low linolenic acid content;
high palmitic acid, high oleic acid, and low linoleic acid content;
high palmitic acid, high oleic acid, and low linolenic acid content;
high palmitic acid, high oleic acid, low linoleic acid, low linolenic acid content; or
high stearic acid, high oleic acid, low linoleic acid, and low linolenic acid content,
relative to oil produced from a control population of soybean plants or seeds.

37. The oil of claim 36, comprising high saturated fatty acid to unsaturated fatty acid composition relative to oil produced from a control population of soybean plants or seeds.

38. The oil of claim 36, comprising high saturated plus monounsaturated fatty acids to polyunsaturated fatty acid composition relative to oil produced from a control population of soybean plants or seeds.

39. The oil of any one of claims 36-38, comprising at least about 4% increase in palmitic acid content or at least about 0.5% increase in stearic acid content relative to oil produced from a control population of soybean plants or seeds.

40. The oil of any one of claims 36-39, comprising a palmitic acid content of about 15% to about 30% and/or a stearic acid content of about 2.5% to about 3.5%.

41. The oil of any one of claims 36-40, comprising a palmitic acid content of about 15% to about 30%, an oleic acid content of about 35% to about 80%, a linoleic acid content of about 5% to 25%, and/or a linolenic acid content of about 1% to about 5%.

42. Soybean oil comprising a palmitic acid content of about 15% to about 30% and an oleic acid content of about 35% to about 80%.

43. The soybean oil of claim 47, comprising a linoleic acid content of about 5% to about 25%.

44. The soybean oil of claim 47 or 48, comprising a linolenic acid content of about 1% to about 5%.

45. The oil of any one of claims 36-44, comprising at least one saturated quantitative trait locus (QTL) associated with high palmitic acid or high stearic acid content and/or at least one unsaturated QTL associated with high oleic acid, low linoleic acid, and/or low linolenic acid content.

46. The oil of claim 45, wherein the at least one saturated QTL is Gm08:063500, Gm08:045000, Gm08:057400, Gm08:072100, Gm08:083900, Gm08:084300, Gm08:092100 and/or Gm08:126400, and the at least one unsaturated QTL is Gm10:50014440, Gm20:35318088, Gm14:45937922, Gm14:45937935, and/or Gm02:41422213.

47. The oil of claim 45 or 46, wherein said at least one saturated QTL comprises at least one saturated SNP marker, and said at least one unsaturated QTL comprises at least one unsaturated SNP marker.

48. The oil of claim 47, wherein the at least one saturated SNP marker is a G or an A at position 4879302, a T or a C at position 3567986, a T or a C at position 4416970, an A or a T at position 5521970, an A or a T at position 6333332, a T or a C at position 6357981, a T or a C at position 6958927, or an A or a G at position 9738629 of chromosome 8; and wherein the at least one unsaturated SNP marker is an A or a G at position 50014440 of chromosome 10, a G or a C at position 35318088 of chromosome 20, an A or a G at position 45937922 of chromosome 14, an A or a G at position 45937935 of chromosome 14, and/or an A or a G at position 41422213 of chromosome 2 of the soybean genome,
wherein the G at position 4879302, the T at position 3567986, the T at position 4416970, the A at position 5521970, the A at position 6333332, the T at position 6357981, the T at position 6958927, and/or the A at position 9738629 of chromosome 8 is associated with high palmitic acid content; and the A at position 50014440 of chromosome 10, the G at position 35318088 of chromosome 20, the A at position 45937922 of chromosome 14, the A at position 45937935 of chromosome 14, and/or the A at position 41422213 of chromosome 2 is associated with high oleic acid, low linoleic acid, and/or low linolenic acid content.

49. A nucleic acid molecule for detecting a molecular marker in a soybean genome or oil associated with high palmitic acid, high oleic acid, low linoleic acid, and/or low linolenic acid content, wherein the nucleic acid molecule comprises at least 15 nucleotides and has at least 90% sequence identity to a sequence of the same number of contiguous nucleotides of a sense or antisense DNA strand in a region comprising or adjacent to the molecular marker, wherein the molecular marker is located in a genomic region 3567986-9738629 of chromosome 8, a genomic region 50013483-50015460 of chromosome 10, a genomic region 35315629-35319063 of chromosome 20, a genomic region 45935667-45939896 of chromosome 14, or a genomic region 41419655-41423881 of chromosome 2 of a soybean genome.

50. The nucleic acid molecule of claim 49, wherein the molecular marker is a single nucleotide polymorphism (SNP) marker, and wherein the SNP marker is selected from the group consisting of a G or an A at position 4879302, a T or a C at position 3567986, a T or a C at position 4416970, an A or a T at position 5521970, an A or a T at position 6333332, a T or a C at position 6357981, a T or a C at position 6958927, and an A or a G at position 9738629 of chromosome 8; an A or a G at position 50014440 of chromosome 10; a G or a C at position 35318088 of chromosome 20; an A or a G at position 45937922 of chromosome 14; an A or a G at position 45937935 of chromosome 14; and/or an A or a G at position 41422213 of chromosome 2 of the soybean genome.

51. The nucleic acid molecule of claim 51, wherein said nucleic acid molecule comprises any one of SEQ ID NOs: 1, 2, 5, 6, 9, 10, 13, 14, 17, 18, 21, 22, 25, 26, 29, 30, 33, 34, 37, 38, 41, 42, 45, 46, 49, and 50.

52. The nucleic acid molecule of any one of claims 49-51, further comprising a detectable label.

53. The nucleic acid molecule of claim 52, wherein said detectable label is a fluorescent label or a radioactive label.


SEQUENCE LISTING (52 sequences)

SEQ ID NO: 1 - DNA, 17 bp, synthetic construct
  source 1..17: mol_type = other DNA
actttggtgagttgtgg

SEQ ID NO: 2 - DNA, 19 bp, synthetic construct
  source 1..19: mol_type = other DNA
ttcactttgatgagttgtg

SEQ ID NO: 3 - DNA, 26 bp, synthetic construct
  source 1..26: mol_type = other DNA
actatcaacaagggtggaaaactgta

SEQ ID NO: 4 - DNA, 28 bp, synthetic construct
  source 1..28: mol_type = other DNA
cgacatcttcagtatgatttaccctatt

SEQ ID NO: 5 - DNA, 16 bp, synthetic construct
  source 1..16: mol_type = other DNA
ctgaaagaggcatttt

SEQ ID NO: 6 - DNA, 16 bp, synthetic construct
  source 1..16: mol_type = other DNA
ctgaaagaggcacttt

SEQ ID NO: 7 - DNA, 26 bp, synthetic construct
  source 1..26: mol_type = other DNA
caagagtgtccattatcaggtttacc

SEQ ID NO: 8 - DNA, 26 bp, synthetic construct
  source 1..26: mol_type = other DNA
caactttgtagctgctgtttcttctg

SEQ ID NO: 9 - DNA, 19 bp, synthetic construct
  source 1..19: mol_type = other DNA
tcctaccctacctagttag

SEQ ID NO: 10 - DNA, 18 bp, synthetic construct
  source 1..18: mol_type = other DNA
tcctaccctacctagcta

SEQ ID NO: 11 - DNA, 22 bp, synthetic construct
  source 1..22: mol_type = other DNA
gctccactgtccagcaaataaa

SEQ ID NO: 12 - DNA, 20 bp, synthetic construct
  source 1..20: mol_type = other DNA
ggatggcatgagcaaattga

SEQ ID NO: 13 - DNA, 17 bp, synthetic construct
  source 1..17: mol_type = other DNA
tcaagttgtaatagctg

SEQ ID NO: 14 - DNA, 19 bp, synthetic construct
  source 1..19: mol_type = other DNA
caagttgtaattgctgaag

SEQ ID NO: 15 - DNA, 18 bp, synthetic construct
  source 1..18: mol_type = other DNA
ggcgcaaccacctatgct

SEQ ID NO: 16 - DNA, 24 bp, synthetic construct
  source 1..24: mol_type = other DNA
gcattgtgcatgtgatgatatagc

SEQ ID NO: 17 - DNA, 14 bp, synthetic construct
  source 1..14: mol_type = other DNA
catgtcgccacatc

SEQ ID NO: 18 - DNA, 14 bp, synthetic construct
  source 1..14: mol_type = other DNA
catgtcgcctcatc

SEQ ID NO: 19 - DNA, 26 bp, synthetic construct
  source 1..26: mol_type = other DNA
tggacacaatttgtttgttaatgatg

SEQ ID NO: 20 - DNA, 29 bp, synthetic construct
  source 1..29: mol_type = other DNA
gcattaggtagatgtggaatattttcttg

SEQ ID NO: 21 - DNA, 17 bp, synthetic construct
  source 1..17: mol_type = other DNA
cacatgcaatttccact

SEQ ID NO: 22 - DNA, 14 bp, synthetic construct
  source 1..14: mol_type = other DNA
acatgcaacttcca

SEQ ID NO: 23 - DNA, 26 bp, synthetic construct
  source 1..26: mol_type = other DNA
aaattagcaaagtgaaagcatagcaa

SEQ ID NO: 24 - DNA, 24 bp, synthetic construct
  source 1..24: mol_type = other DNA
aattttgatgccagaagtgtcctt

SEQ ID NO: 25 - DNA, 14 bp, synthetic construct
  source 1..14: mol_type = other DNA
ctgctgccctgtca

SEQ ID NO: 26 - DNA, 14 bp, synthetic construct
  source 1..14: mol_type = other DNA
tgctgccctgccat

SEQ ID NO: 27 - DNA, 22 bp, synthetic construct
  source 1..22: mol_type = other DNA
tccttaacacagttgcgattgc

SEQ ID NO: 28 - DNA, 21 bp, synthetic construct
  source 1..21: mol_type = other DNA
caataccacccaacacaagca

SEQ ID NO: 29 - DNA, 20 bp, synthetic construct
  source 1..20: mol_type = other DNA
ataatagaggaataagcaac

SEQ ID NO: 30 - DNA, 20 bp, synthetic construct
  source 1..20: mol_type = other DNA
ataatagaggaataggcaac

SEQ ID NO: 31 - DNA, 25 bp, synthetic construct
  source 1..25: mol_type = other DNA
aaacaggccaaacaataacaaattc

SEQ ID NO: 32 - DNA, 25 bp, synthetic construct
  source 1..25: mol_type = other DNA
catgataggttcttgcaagtttacg

SEQ ID NO: 33 - DNA, 15 bp, synthetic construct
  source 1..15: mol_type = other DNA
catgccttcaacaag

SEQ ID NO: 34 - DNA, 15 bp, synthetic construct
  source 1..15: mol_type = other DNA
catgccttcagcaag

SEQ ID NO: 35 - DNA, 20 bp, synthetic construct
  source 1..20: mol_type = other DNA
ctggtgtgtgggtgattgct

SEQ ID NO: 36 - DNA, 28 bp, synthetic construct
  source 1..28: mol_type = other DNA
aaataagggactaaaagtgttgagtgaa

SEQ ID NO: 37 - DNA, 18 bp, synthetic construct
  source 1..18: mol_type = other DNA
cttttagtccgttatttc

SEQ ID NO: 38 - DNA, 18 bp, synthetic construct
  source 1..18: mol_type = other DNA
cttttagtcccttatttc

SEQ ID NO: 39 - DNA, 22 bp, synthetic construct
  source 1..22: mol_type = other DNA
tcaccatgccttcagcaagtac

SEQ ID NO: 40 - DNA, 19 bp, synthetic construct
  source 1..19: mol_type = other DNA
cggcgatggcttattttcc

SEQ ID NO: 41 - DNA, 15 bp, synthetic construct
  source 1..15: mol_type = other DNA
ggcaagataacaaaa

SEQ ID NO: 42 - DNA, 15 bp, synthetic construct
  source 1..15: mol_type = other DNA
gcaaggtaacaaaaa

SEQ ID NO: 43 - DNA, 23 bp, synthetic construct
  source 1..23: mol_type = other DNA
ggtcatcatcagaaactgccttg

SEQ ID NO: 44 - DNA, 33 bp, synthetic construct
  source 1..33: mol_type = other DNA
aaggtagtatctaacatttaagtgttcactcac

SEQ ID NO: 45 - DNA, 14 bp, synthetic construct
  source 1..14: mol_type = other DNA
ctgccttgatatcg

SEQ ID NO: 46 - DNA, 14 bp, synthetic construct
  source 1..14: mol_type = other DNA
ctgccttggtatcg

SEQ ID NO: 47 - DNA, 25 bp, synthetic construct
  source 1..25: mol_type = other DNA
ctttgtcacatacttgcatcaccat

SEQ ID NO: 48 - DNA, 33 bp, synthetic construct; identical to SEQ ID NO: 44
  source 1..33: mol_type = other DNA

SEQ ID NO: 49 - DNA, 20 bp, synthetic construct
  source 1..20: mol_type = other DNA
gaattccatattggataact

SEQ ID NO: 50 - DNA, 18 bp, synthetic construct
  source 1..18: mol_type = other DNA
ccatattgggtaactaaa

SEQ ID NO: 51 - DNA, 28 bp, synthetic construct
  source 1..28: mol_type = other DNA
ccatgttttctctgcttatctatctctc

SEQ ID NO: 52 - DNA, 28 bp, synthetic construct
  source 1..28: mol_type = other DNA
aaaaaggaggaaaaagtaacaatgtagg